(19) United States
(12) Patent Application Publication
(10) Pub. No.: US 2022/0040310 A1
(43) Pub. Date: Feb. 10, 2022

(54) Methods, agents and compositions as in situ vaccine for cancer cell and tumor treatment

(71) Applicant: Tianxin Wang, Walnut Greek, CA (US)

(72) Inventor: Tianxin Wang, Walnut Creek, CA (US)

(73) Assignee: Tianxin Wang, Walnut Creek, CA (US)

(21) Appl. No.: 17/509,028

(22) Filed: Oct. 24, 2021

Related U.S. Application Data
(63) Continuation-in-part of application No. 15/945,741, filed on Apr. 5, 2018.
Application No. 15/945,741 is a continuation-in-part of application No. 16/271,877, filed on Feb. 10, 2019.
Application No. 16/271,877 is a continuation-in-part of application No. 15/945,741, filed on Apr. 5, 2018.
Application No. 15/945,741 is a continuation-in-part of application No. 16/924,184, filed on Jul. 9, 2020.
(60) Provisional application No. 63/108,409, filed on Nov. 1, 2020.
(60) Provisional application No. 63/118,739, filed on Nov. 26, 2020.
(60) Provisional application No. 63/121,912, filed on Dec. 5, 2020.
(60) Provisional application No. 63/129,489, filed on Dec. 22, 2020.
(60) Provisional application No. 62/482,982, filed on Apr. 7, 2017.
(60) Provisional application No. 62/631,616, filed on Feb. 16, 2018.
(60) Provisional application No. 62/636,838, filed on Feb. 28, 2018.
(60) Provisional application No. 62/649,579, filed on Mar. 29, 2018.
(60) Provisional application No. 62/873,969, filed on Jul. 14, 2019.

(51) Int. Cl. A61K 47/34; A61P 35/00; A61K 9/00; A61K 39/395; A61K 31/4745; A61K 31/713

(52) U.S. Cl. CPC A61K 47/34; A61P 35/00; A61K 9/0019; A61K 39/3955; A61K 31/4745; A61K 31/713; A61K 39/39558

(57) ABSTRACT

This disclosure provides agents, compositions and methods for treating cancer by treating tumor in a subject. The composition comprises cancer cell inactivating agent and immune activity enhancing agent in a sustained release formulation, which can be used as intratumoral injection to convert the treated tumor into an in situ vaccine for cancer. Suitable immune activity enhancing agents include TLR agonist and STING agonist.

### CROSS-REFERENCE TO RELATED APPLICATION

This application claim priority to U.S. Provisional Patent Applications 63/108,409 filed on Nov. 1, 2020, 63/118,739 filed on Nov. 26, 2020, 63/121,912 filed on Dec. 5, 2020 and 63/129,489 filed on Dec. 22, 2020. It is also a Continuation-In-Part application of U.S. application Ser. No. 15/945,741, filed on Apr. 5, 2018, U.S. application Ser. No. 16/271,877, filed on Feb. 10, 2019, and U.S. application Ser. No. 16/924,184, filed on Jul. 9, 2020. The entire disclosure of the prior applications is considered to be part of the disclosure of the instant application and is hereby incorporated by reference.

### FIELD

This disclosure provides compounds (agents), compositions and methods for treating cancer by treating and/or inhibiting tumors in a subject. The composition comprises cancer cell inactivating agent and immune activity enhancing agent in a sustained release formulation, which can be used as intratumoral injection to convert the treated tumor into a in situ vaccine to treat cancer.

### BACKGROUND

As cancer immunotherapy continues to benefit from novel approaches such as checkpoint inhibitors there will be increasing need to develop cancer vaccines to guide the immune system specifically toward tumor antigens including tumor associated antigen and tumor neoantigen. In situ vaccination represents an alternative approach in which the cancer vaccine is generated in vivo without the need to previously identify and isolate the tumor antigens. Herein, in situ vaccination refers to any approach which exploits tumor antigen available at a tumor site to induce antigen specific adaptive immune response. In situ vaccination is a promising strategy for cancer immunotherapy owing to its convenience and the ability to induce numerous tumor antigens. However, the advancement of in situ vaccination techniques has been hindered by low cross-presentation of tumor antigens and the immunosuppressive tumor microenvironment. Researchers continue to search for improved treatment for patients with advanced cancers are needed.

### SUMMARY

The present disclosure is directed to compounds (agents), compositions and methods for treating cancer by treating and/or inhibiting tumors in a subject in need such as a cancer patient. The current invention relates to novel methods and agents as in situ vaccine to treat cancer. In some embodiments, the novel agents are in the form of antibody binding molecule-cell surface anchoring molecule conjugate that facilitates the lysis of cancer cells and/or antigen presenting using exogenous antibody. The antibody binding molecule-cell surface anchoring molecule conjugate that can enhance the killing of cancer cells and/or antigen presenting is called cancer cell inactivating agent. Also provided are pharmaceutical compositions comprising an antibody binding conjugate, such as, but not limited to, those described herein, and a Toll-like receptor (TLR) agonist or STING agonist. Suitable Toll-like receptor (TLR) agonists include, but are not limited to, CpG ODN (CpG oligodeoxynucleotide), polyinosinic:polycytidylic acid (poly IC), imiquimod, and the like, or a mixture thereof in a sustained release formulation such as in-situ gelling system or implant. In certain embodiments, the present disclosure is directed to a method of treating and/or inhibiting a tumor and its metastasis, comprising administering to a patient in need thereof a therapeutically effective amount of an antibody binding conjugate or a pharmaceutical composition as described herein. U.S. patent application Ser. Nos. 16/271,877 and 15/945,741 disclosed agents, composition, formulation and method to treat tumor cell and cancer, therapeutically effective amount STING agonist can be added to the examples and embodiments, compositions, formulations and methods disclosed in these applications to treat tumor cells and cancer. In certain embodiments, the present disclosure is directed to a method of treating and/or inhibiting a tumor and its metastasis, comprising administering to a patient in need thereof a therapeutically effective amount of a cell surface anchoring antigen conjugate or a pharmaceutical composition as described herein. Furthermore, the cell surface anchoring antigen conjugate can further comprise a TLR agonist moiety or STING agonist moiety.

### BRIEF DESCRIPTION OF THE DRAWINGS

FIG. 1 shows examples of antibody having tandem format of Fc.

FIG. 2 shows another format of antibody having two copy of native or engineered Fc.

FIG. 3 shows examples of antibody having CH2 repeats.

FIG. 4 shows exemplary structures of an IgM pentamer targeting tumor antigen.

FIG. 5 shows exemplary structures of monospecific or bispecific antibody having tandem format of Fc.

FIG. 6 shows exemplary structures of monospecific or bispecific antibody having CH2 repeat.

FIG. 7 shows examples of the TLR agonist moiety in the antibody conjugate.

FIG. 8 shows examples of TLR agonist or STING agonist moiety in ADC.

FIG. 9 shows examples of TLR agonist or STING agonist conjugated to antibodies having tandem Fc or repeat CH2.

FIG. 10 shows exemplary formats of TLR agonist conjugated to bispecific antibody.

FIG. 11 shows multiple antigen, cell anchoring molecule and TLR agonist are conjugated to a soluble polymer backbone.

FIG. 12 shows examples of conjugate using hyaluronic acid (HA) based backbone.

FIG. 13 shows examples of conjugate using lysine containing peptide as backbone.

FIG. 14 shows examples of conjugate using Glu containing peptide as backbone.

FIG. 15 shows an example of conjugate using Lys/Glu containing peptide as backbone and STING agonist.

FIG. 16 shows an example of TLR agonist that can replace the STING agonist in the conjugate shown in FIG. 15.

FIG. 17 shows an example of conjugate using Lys containing peptide as backbone and fatty acid as lipid.

FIG. 18 shows an example of conjugate having 4 antigens.

### DETAILED DESCRIPTION

It is to be noted that as used herein and in the appended claims, the singular forms “a”, “an”, and “the” include plural referents unless the context clearly dictates otherwise. Thus, for example, reference to “an adjuvant” includes a plurality of adjuvants.

The term “about” when used before a numerical designation, e.g., temperature, time, amount, and concentration, including range, indicates approximations which may vary by (+) or (−) 30%, 15% or 5%.

As used herein, the term “treating” refers to preventing, curing, reversing, attenuating, alleviating, minimizing, inhibiting, suppressing and/or halting a disease or disorder, including one or more clinical symptoms thereof.

As used herein, the term “composition” refers to a preparation suitable for administration to an intended patient for therapeutic purposes that contains at least one pharmaceutically active ingredient, including any solid form thereof. In certain embodiments, the composition is formulated as an injectable formulation. In certain embodiments, the composition is formulated as a film, gel including in situ gelling formulation, or liquid solution including suspension.

As used herein, the term “pharmaceutically acceptable” indicates that the indicated material does not have properties that would cause a reasonably prudent medical practitioner to avoid administration of the material to a patient, taking into consideration the disease or conditions to be treated and the respective route of administration. For example, it is commonly required that such a material be essentially sterile.

As used herein, the term “pharmaceutically acceptable carrier” refers to pharmaceutically acceptable materials, compositions or vehicles, such as a liquid or solid filler, diluent, excipient, solvent or encapsulating material, involved in carrying or transporting any supplement or composition, or component thereof, from one organ, or portion of the body, to another organ, or portion of the body, or to deliver an agent to the desired tissue or a tissue adjacent to the desired tissue.

As used herein, the term “formulated” or “formulation” refers to the process in which different chemical substances, including one or more pharmaceutically active ingredients, are combined to produce a dosage form. In certain embodiments, two or more pharmaceutically active ingredients can be co-formulated into a single dosage form or combined dosage unit, or formulated separately and subsequently combined into a combined dosage unit. A sustained release formulation is a formulation which is designed to slowly release a therapeutic agent in the body over an extended period of time.

As used herein, the term “solution” refers to solutions, suspensions, emulsions, drops, ointments, liquid wash, sprays, liposomes which are well known in the art. In some embodiments, the liquid solution contains an aqueous pH buffering agent which resists changes in pH when small quantities of acid or base are added. In certain embodiments, the liquid solution contains a lubricity enhancing agent.

As used herein, the term “pH buffering agent” refers to an aqueous buffer solution which resists changes in pH when small quantities of acid or base are added to it. pH Buffering solutions typically comprise of a mixture of weak acid and its conjugate base, or vice versa. For example, pH buffering solutions may comprise phosphates such as sodium phosphate, sodium dihydrogen phosphate, sodium dihydrogen phosphate dihydrate, disodium hydrogen phosphate, disodium hydrogen phosphate dodecahydrate, potassium phosphate, potassium dihydrogen phosphate and dipotassium hydrogen phosphate; boric acid and borates such as, sodium borate and potassium borate; citric acid and citrates such as sodium citrate and disodium citrate; acetates such as sodium acetate and potassium acetate; carbonates such as sodium carbonate and sodium hydrogen carbonate, etc. pH Adjusting agents can include, for example, acids such as hydrochloric acid, lactic acid, citric acid, phosphoric acid and acetic acid, and alkaline bases such as sodium hydroxide, potassium hydroxide, sodium carbonate and sodium hydrogen carbonate, etc. In some embodiments, the pH buffering agent is a phosphate buffered saline (PBS) solution (i.e., containing sodium phosphate, sodium chloride and in some formulations, potassium chloride and potassium phosphate).

As used herein, “invention” and “inventions” are interchangeable. the term “invention” refers to all the inventions in the current application. In the current application the “/” mark means both “and” and “or”. As used herein, “%” is w/w based unless specified.

The term “antibody” in the current application include both full antibody and antibody fragment as well as bispecific antibody and multi specific antibody.

As employed herein, the phrase “an effective amount,” refers to a dose sufficient to provide concentrations high enough to impart a beneficial effect on the recipient thereof without unacceptable toxicity. The specific therapeutically effective dose level for any particular subject will depend upon a variety of factors including the disorder being treated, the severity of the disorder, the activity of the specific compound, the route of administration, the rate of clearance of the compound, the duration of treatment, the drugs used in combination or coincident with the compound, the age, body weight, sex, diet, tumor size and general health of the subject, and like factors well known in the medical arts and sciences. Various general considerations taken into account in determining the “therapeutically effective amount” are known to those of skill in the art and are described. Dosage levels typically fall in the range of about 0.001 up to 10 mg/kg; with levels in the range of about 0.001 up to 5 mg/kg are generally applicable.

The current invention relates to novel compounds such as cell surface anchoring antigen conjugates comprising 3β-cholesterylamine, or an analogue or derivative thereof, as those described in U.S. patent application Ser. Nos. 15/945,741, 16/271,877 and 16/924,184. The cell surface anchoring antigen conjugate works as a cancer cell lysing agent and enhances tumor antigen presentation. The cell surface anchoring antigen conjugate is also called cell surface anchoring conjugate, which work as cancer cell inactivating agent. The antigen used in the conjugate can be any antigen, and is in certain embodiments, a molecule that is the antigen of existing antibody in a patient or antigen of TCR (T-cell receptor) of T cell in a patient, which is referred to as a native antigen. Suitable native antigens include, but are not limited to, galactose-alpha-1,3-galactose (α-gal), L-rhamnose, Forssman disaccharide, phosphorylcholine (PC), DNP (dinitrophenyl), or a combination thereof. In some embodiments, the cell surface anchoring antigen conjugate of the current invention has the following formula, which is a conjugate of native antigen with cell surface anchoring molecule via an optional linker: native antigen-optional linker-cell surface anchoring molecule, as described in U.S. patent application Ser. No. 15/945,741.

The cancer cell inactivating agent, formulation or pharmaceutical composition as described in the current application can be injected intratumorally to treat the cancer or injected into or proximal to the tumor draining lymph node to treat cancer or applied to the site where tumor is removed during surgery. The conjugate can further comprise a cancer cell binding moiety to increase its targeting to cancer cell, which will allow intravenous (iv) injection or intramuscular (IM) or subcutaneous (SC) instead of intratumoural injection.

The current invention discloses methods to treat tumor cell and cancer and to boost immunity against tumor cell. The method comprises giving patient said cancer cell inactivating agent and/or agent can enhance cancer cell antigen presenting or in combination with an immune activity enhancing agent (immunity boosting agent) and exogenous antibody that can bind with the cancer cell inactivating agent if cancer cell inactivating agent is not a native antigen. Examples of suitable cell surface anchoring conjugate (cancer cell inactivating agent) that require exogenous antibody are disclosed in U.S. patent application Ser. Nos. 16/271,877 and 16/924,184. In addition to above treatment regiment, immune checkpoint inhibitors at therapeutical effective amount could be given to further enhance this treatment systematically or co-formulated together to be intratumorally injected into or proximal to the tumor draining lymph node or applied to the site where tumor is removed during surgery. In some embodiments, the immune activity enhancing agent (immunity boosting agent) is also called vaccine adjuvant type agent. In some embodiments the immune activity enhancing agent is given by intratumoural injection. It can be given to the patient by intratumoural injection as a mixture with the said cancer cell inactivating agent/agent can enhance cancer cell antigen presenting or sequentially (before or after) to the same tumor injected with the cancer cell inactivating agent. For example, a solution formulation containing both said cancer cell inactivating agent and/or agent that can enhance cancer cell antigen presenting and immunity boosting agent can be injected into the tumor at 5 μL˜1000 μL/cm³ tumor volume or 100 μL˜5 mL/tumor. Suitable tumor can be any type of solid tumor as long as it allows intratumoral injection. The antibody that can bind with the cancer cell inactivating agent and/or agent can enhance cancer cell antigen presenting can be given by intratumoural injection (e.g. 0.5˜50 mg/cm³ tumor volume or 1 mg-100 mg/tumor) or be given systematically at the same time or within ±3 weeks. Examples of these antibody are recombinant therapeutic antibodies used for cancer treatment. The antibody can be given by intratumoural injection including IV, IM and SC injection together with the immune activity enhancing agent.

Also provided are methods of inhibiting or eliminating cancer cells in a tumor and/or preventing metastasis. The method comprises administering to a patient in need thereof a formulation or composition as described herein, which comprises a cancer cell lysing agent, such as cell surface anchoring conjugate or antibody against tumor cell surface antigen, in combination with an immune function enhancing agent in a sustained release formulation such as in-situ gelling system or implant. The composition may be administered via intratumoral injection to the tumor or injected into or proximal to the tumor draining lymph node or applied to the site where tumor is surgically removed. The immune function enhancing agent can be given to the patient by intratumoral injection as a mixture with the cancer cell lysing agent, such as a cell surface anchoring antigen conjugate or antibody against tumor cell surface antigen, or sequentially (before or after) to the same tumor injected with a cancer cell lysing agent. For example, a liquid formulation containing both a cancer cell lysing agent and an immune function enhancing agent can be injected into the tumor (e.g., at 5 μL˜1000 μL/cm³ tumor volume or 100 μL˜5 mL/tumor). The tumor be any type of solid tumor, provided it allows intratumoral injection.

In summary, provided are methods to kill cancers cells in a tumor and/or to prevent or delay metastasis by treating a primary tumor. The method comprises administering to a patient in need thereof, a cancer cell lysing agent optionally in combination with an immune function enhancing agent, which essentially convert the tumor into a in situ vaccine. Immune checkpoint inhibitors at therapeutically effective amounts can also be administered to further enhance this treatment. The immune function enhancing agent is administered by intratumoral injection to the primary tumor or injected into or proximal to the tumor draining lymph node. It can be administered to a subject in need thereof by intratumoral injection as a mixture with a cancer cell lysing agent or sequentially (before or after) to the same tumor injected with the cancer cell lysing reagent. The treatment to the primary tumor will induce an immune response against distant and secondary tumor to kill the cancer cells within, as well as prevent the metastasis of tumor. The composition and formulation used for intratumoral injection comprises a cancer cell lysing agent and an immune function enhancing agent in a pharmaceutical acceptable carrier preferably in a sustained release formulation such as in-situ gelling system or implant. It can be injectable liquid or solid dosage form, such as a lyophilized formulation, that can be reconstituted with an injectable liquid. The cancer cell lysing agent and immune function enhancing agent can be in the form of an active drug, prodrug, liposome, micelle, emulsion, gel including in situ gelling system, implant, thermal phase changing formulation, insoluble precipitate (e.g. in complex with condensing reagent), conjugated to polymer drug carrier (e.g. dextran), coated on the surface or encapsulated within biodegradable micro particle or nanoparticle. A thermal phase changing formulation is a formulation that changes its phase from a liquid to a semisolid when the temperature increases. Such formulations typically use poloxamer as an excipient. Exemplary sizes of the microparticles or nanoparticles is between 10 nm and 100 μm. In some embodiments, the particle is alginate based such as alginate-calcium particle.

The current invention also discloses novel compositions/formulations to treat tumor cell and cancer and to boost immunity. The compositions/formulation comprises said cancer cell inactivating agent and/or agent can enhance cancer cell antigen presenting and immune activity enhancing agent in a pharmaceutical acceptable carrier preferably in a sustained release formulation such as in-situ gelling system or implant or nano/micro particle. It can be injectable solution or solid dosage form such as lyophilized formulation that can be reconstituted to injectable solution. The formulation contains cancer cell inactivating agent and/or agent can enhance cancer cell antigen presenting and immune activity enhancing agent as well as pharmaceutical acceptable excipients suitable for injection such as buffering salt (e.g. PBS salt), amino acid, carbohydrate (e.g. mannose, trehalose) and surfactant (e.g. PEG, tween, PVA, lethicin) or their combination. The formulation can further comprise antibody that can bind with the cancer cell inactivating agent/agent that can enhance cancer cell antigen presenting.

In some embodiments, the compositions/formulation comprises TLR agonist and/or STING agonist in a pharmaceutical acceptable carrier with therapeutical antibody against cancer cell antigen, preferably in a sustained release formulation such as in-situ gelling system or implant. It can be injected into tumor to treat cancer, or injected into or proximal to the tumor draining lymph node to treat cancer or applied to the site where tumor is surgically removed.

It can be in a sustained release formulation such as micro/nano particle form or gel or high viscosity liquid or in situ gelling system. Examples of therapeutical antibody for cancer can be the current therapeutical antibody drugs used clinically, such as Herceptin. It can be used for cancer with low HER2 expression and improve Herceptin's efficacy if trastuzumab is used in the composition to be injected in to tumor.

The current invention also discloses methods to boost immunity and kill cancers cells in distant tumor and/or prevent metastasis. The method comprises giving the object in need the said cancer cell inactivating agent and/or agent can enhance cancer cell antigen presenting and/or in combination with an immune activity enhancing agent. The antibody that can bind with the cancer cell inactivating agent/agent can enhance cancer cell antigen presenting is given by intratumoural injection (e.g. 5 μL˜1000 μL/cm³ tumor volume or 100 μL˜5 mL/tumor), or injected into or proximal to the tumor draining lymph node, or be given systematically at the same time or within ±3 weeks. In addition to above treatment regiment, immune checkpoint inhibitors at therapeutical effective amount could be given to further enhance this treatment. The immune activity enhancing agent is given by intratumoural injection to the primary tumor. It can be given to the object in need by intratumoural injection as a mixture with the said cancer cell inactivating agent and/or agent can enhance cancer cell antigen presenting or sequentially (before or after) to the same tumor injected with the cancer cell inactivating agent. The injected cancer cell inactivating agent and/or agent can enhance cancer cell antigen presenting will be present on the cancer cell surface and attract the antibody added. The antibody will produce cancel cell killing effect and/or cancer antigen presenting to immune cells, therefore generate immune response against cancer cells. The treatment to the primary tumor will induce immune response against distant and secondary tumor to kill the cancer cells within, as well as prevent the metastasis of tumor.

Examples of suitable immune check point inhibitors include PD-1 antagonist such as antibody against PD-1, antibody against PD-L1, antibody against CTLA-4, antibody against OX40 or other OX40 agonist, TIM-3 blocking antibody, GITR blocking antibody, LAG3 (CD223) antibody, CD112R antibody or their combinations. Some are commercial available and can be readily used for the current invention such as ipilimumab, tremelimumab, atezolizumab, nivolumab and pembrolizumab. They can be administered to the patient after the cancer cell inactivating agent treatment or co-formulated with the cancer cell inactivating agent. For example, the patient can be intravenously injected with ipilimumab 3˜10 mg/kg every 3 weeks for 4 doses after treatment or atezolizumab 1200 mg IV q3wk after treatment until disease progression. The current treatment dosing of these immune check point inhibitors can be used. They can be also be injected intratumorally, or injected into or proximal to the tumor draining lymph node, where lower than systematic amount can be used. They can be co-formulated with the above cancer cell inactivating agent and/or agent can enhance cancer cell antigen presenting and immune activity enhancing agent; and used as intratumoral injection, or being injected into or proximal to the tumor draining lymph node.

Another method to use the compositions and formulations of the current invention to treat cancer is to apply them during surgery to the sites where tumor is removed or being injected into or proximal to the tumor draining lymph node after surgery. Suitable dose of the active drug (e.g. cancer cell lysing/inactivating agent, agent can enhance cancer cell antigen presenting and immune function enhancing agent) can be same or lower than the dose used for intratumoral injection, such as 5%˜100% of the dose used intratumoral injection. It can be sprayed or dropped or painted onto the tumor removed area if the formulation is liquid. It can also be semi solid (e.g. gel) or solid form such as powder (e.g. lyophilized powder) or film or other implant form that can be applied directly to the open wound area where tumor is removed right after it is removed during surgery. The formulation can be a liquid of self-gelling system. Once it is applied to the surgery area, suitable amount of agent that can trigger gelling can be also added to the same area to promote gelling. For example, if sodium alginate solution (e.g. 1%˜10% w/w) containing drugs is used, calcium salt solution (e.g. CaCl₂) or Ca gluconate 0.5%˜5% w/w) as gelling trigger agent can be used to promote gelling on site. In one example, a formulation containing 5% sodium alginate and 5% antibody against tumor surface antigen is applied to the area where tumor is removed followed by the addition of 5% CaCl₂) to form a gel layer on top of the surgical area. It can also be mixed with a gelling agent to form a gel and then applied to the surgical area, which is essentially an implant. In another example, a formulation containing 3% sodium alginate and 5% antibody against tumor surface antigen is mixed with 10% CaCl₂) solution at 10:1 v/v ratio to form a gel and then the gel is applied to the area where tumor is removed. The gel can also be dried in to a film/membrane and then the resulting membrane/film is applied to the tumor removed site. Examples of antibody include mono specific antibody, bispecific antibody and antibody drug conjugate described in the current application. Examples of antibody can be selected from those clinically used or under development antibody drug against tumor cell surface antigen such as rituximab, Bexxar, Herceptin, panitumumab, zalutumumab, nimotuzumab, matuzumab, pertuzumab, margetuximab, bevacizumab, brentuximab, ado-trastuzumab emtansine, catumaxomab, blinatumomab and antibody against PD-L1, antibody against Trop-2, antibody against folate receptor alpha (FRa), antibody against onco-embryonic antigen ROR1, antibody against EpCAM, antibody against gp100, antibody against mesothelin, antibody against Axl, antibody against CLDN18 such as antibody against Claudin 18.2 (CLDN18.2), antibody against BTN3a, antibody against CD155, antibody against CD112, antibody against CD133, antibody against CD47, antibody against ICAM1, antibody against ALK (anaplastic lymphoma kinase), antibody against BCR-ABL, antibody against c-Met, antibody against RTK (receptor tyrosine kinases), antibody against ROS1, antibody against TRK (tropomyosin-receptor-kinase receptor), antibody against RET. Additional checkpoint inhibitor antibody can also be incorporated in the alginate solution such as those described previously at 1˜5% w/w. In some examples, additional chemotherapy drug can also be incorporated in the alginate solution. Example of these drugs include alkylating agents, antimetabolites, anti-microtubule agents, topoisomerase inhibitors, cytotoxic antibiotics and cisplatin family drugs such as those disclosed in embodiments type 3. The concentration in these chemotherapy drug in the alginate solution can be 0.5%˜10% w/w. Furthermore, additional immune activity enhancing agent such as TLR agonist (e.g. 0.1% poly IC) can also be incorporated in the alginate solution. The self-gelling agent alginate can also be replaced with other self-gelling agent described in the current invention such as poloxamer or PLGA in organic solvent. The intratumoral injected formulations described in the current invention can be readily converted to a gel or implant in vitro and then be applied to the site where tumor is removed during surgery to treat cancer. The gel can also be dried to form a drug containing film or other shape (e.g. disk, plate, membrane) implant and then applied to the tumor removed site.

Examples of suitable immune function/activity enhancing agent (immunity boosting agent) for the current inventions can be found in U.S. patent application Ser. Nos. 15/945,741, 16/271,877 and 16/924,184, include pattern recognition receptor (PRR) ligands, RIG-I-Like receptor (RLR) ligands (e.g. RIG-I agonist, MDA5 agonist, LGP2 agonist), Nod-like receptor (NLR) ligands, C-type lectin receptors (CLR) ligands, STING agonist, and Toll-like receptor ligands such as a TLR3 ligand, TLR4 ligand, TLR5 ligand, TLR7/8 ligand, TLR9 ligand, or a combination thereof. The immune function enhancing agent can be a vaccine adjuvant. Example of suitable vaccine adjuvant can be saponin such as Matrix-M adjuvant (Quillaja saponins formulated with cholesterol and phospholipids into nanoparticles), squalene such as MF59 (an oil-in-water emulsion of squalene oil) and AS03 adjuvant (vitamin E and squalene oil-in-water emulsion), MPL such as AS01B (made of up MPL, a purified fat-like substance), QS-21 which is purified from the bark of the Quillaja Saponaria, AS04 which is a combination of aluminum hydroxide and monophosphoryl lipid A (MPL). MPL is a purified fat-like substance, aluminum salts such as aluminum hydroxide, aluminum phosphate, alum (potassium aluminum sulfate), or mixed aluminum salts. The concentration of these vaccine adjuvants can be the same as currently used concentration or up to 20× higher. Preferably the Toll-like receptor ligand is a Toll-like receptor (TLR) agonist. Additional immune activity enhancing agent (immunity boosting agent) include interleukin 12, tumor necrosis factor, interferon gamma (IFNγ), immunomodulatory imide drugs (IMiDs such as thalidomide, lenalidomide and pomalidomide, Treg inhibitory agent such as inhibitory antibody against Treg or their combinations. Many of them are commercial available (e.g. those listed by Invivogen) and can be readily used for the current invention. Example includes imidazoquinoline family of TLR7/8 ligands (e.g. imiquimod(R837), gardiquimod, resiquimod (R848), 3M-052, 3M-852, 3M-S-34240, CL264, CL401, CL413, CL419, CL553, CL572, CL531, PM2CSK4, PM3CSK4, motolimod/VTX-2337, NKTR-262; CpG ODNs such as SD-101, ODN 1826 and ODN 2216, TLR agonist disclosed in PMID: 32203790, TLR agonist including TLR peptide agonist disclosed in patent applications WO2018055060A1, WO2013120073A1, WO2016146143A1 and US20180133295 and their citations, synthetic analogs of dsRNA, such as poly IC (e.g. Poly ICLC, polylC-kanamycin, PolyI:PolyC12U), TLR4/5 Ligands such as Bacterial lipopolysaccharides (LPS, e.g. monophosphoryl lipid A), bacterial flagellin (e.g. vibrio vulnificus flagellin B), glucopyranosyl lipid A (GLA), TLR7 agonist loxoribine or their derivatives/analogues, or their combinations. They can be in form of active drug, prodrug, liposome, emulsion, micelle, insoluble precipitate (e.g. in complex with condensing agent), conjugated to polymer drug carrier (e.g. dextran) or encapsulated in biodegradable micro particle/nano particle (e.g. those made of biodegradable polymer such as PLA, PLGA, PCL, PGA, PLHMGA, prolifeprospan such as prolifeprospan20, or PHB or alginate based such as alginate-calcium particle). The use and preparation of vaccine adjuvant encapsulated micro particle/nano particle or its prodrug are well known to the skilled in the art. Examples of them suitable for the current invention can be found in or adopted from those disclosed in U.S. patent application Ser. Nos. 15/945,741, 16/271,877 and 16/924,184 and their related citations. Poly acrylic acid containing polymer such as carbomer is also an immune function enhancing agent that can be used. In one example, PLGA-R837 (R837 encapsulated in poly lactide-co-glycolide particles) nanoparticle are prepared using o/w single-emulsion method as that disclosed in U.S. patent application Ser. No. 16/271,877. In another example, PLGA-R837/ADU-S100 (R837 and ADU-S100 encapsulated in poly lactide-co-glycolide particles) nanoparticle are prepared using o/w single-emulsion method similar to that disclosed U.S. patent application Ser. No. 16/924,184. Briefly, R837 (TLR7 ligand) and STING agonist ADU-S100 are dissolved in DMSO at 1 mg/ml for each. A total of 50 μL above R837/ADU-S100 solution is added to 1 ml PLGA (5 mg/ml) dissolved in dichloromethane. Next the mixture is homogenized with 0.4 ml 5% w/v PVA solution for 10 min using ultrasonication. The o/w emulsion is added to 2.1 ml of a 5% w/v solution of PVA to evaporate the organic solvent for 4 h at room temperature. PLGA-R837/ADU-S100 nanoparticles are obtained after centrifugation at 3,500 g for 20 min. Combination of vaccine adjuvant (immune activity enhancing agent) and cancer cell inactivating agent can also be encapsulated together in micro/nano particles. For example, R837 or R848 or SB 11285 is dissolved in DMSO at 1 mg/ml. A cancer cell inactivating agent of the current invention is dissolved in DMSO at 50 mg/ml. 50 μl R837 or R848 or SB 11285 and 50 μl cancer cell inactivating agent solutions in DMSO are added to 1 ml mPEG-PLGA (10 mg/ml) dissolved in acetonitrile. Next, the mixture was dropwise added into 5 ml water containing 100 mg poly IC. After 1 h stirring and 12 h standing, the nanoparticles are obtained after centrifugation at 22,000 g for 5 min. Preferably the immune activity enhancing agent (immunity boosting agent) is given intratumorally or injected into or proximal to the tumor draining lymph node or applied to the site where tumor is removed during surgery at therapeutical effective amount. For example, the imiquimod can be given at the amount between 50 μg˜5 mg as free drug or given as 1 mg˜100 mg micro or nano particle encapsulating 50 μg˜5 mg imiquimod. For example, the STING agonist can be given at the amount between 10 μg˜5 mg as free drug or given as 1 mg˜100 mg micro or nano particle encapsulating 10 μg˜5 mg STING agonist. Other suitable dosing can be used, as long as it can produce satisfactory therapeutical effect, which can be determined experimentally by screening and testing with well-known protocol and methods. In some embodiments, the intratumorally delivered imiquimod in the composition/formulation is given at the amount between 50 μg˜2 mg/tumor as free drug (free here means it is not in a sustained release form) or given at the amount of 200 μg˜10 mg/tumor when it is encapsulated in micro/nano particle form or in other sustained release formulation. In some embodiments, the intratumorally delivered imiquimod in the composition/formulation is given at the amount between 50 μg˜1 mg/tumor as free drug or given at the amount of 200 μg˜5 mg/tumor when it is encapsulated in micro/nano particle form or in other sustained release formulation. In some embodiments, the intratumorally delivered imiquimod in the composition/formulation of the current invention is given at the amount between 50 μg˜500 μg/tumor as free drug or given at the amount of 200 μg˜2.5 mg/tumor when it is encapsulated in micro/nano particle form or in other sustained release formulation. In some embodiments, the intratumorally delivered imiquimod in the composition/formulation is given at the amount of 200 μg/tumor as free drug or given at the amount of 500 μg/tumor when it is encapsulated in micro/nano particle form or in other sustained release formulation.

In some embodiments, the intratumorally delivered R848 (resiquimod) or gardiquimod or 3M852A in the composition/formulation of the current invention is given at the amount between 30 μg˜1 mg/tumor as free drug (free here means it is not in a sustained release form) or given at the amount of 100 μg˜5 mg/tumor when it is encapsulated in micro/nano particle form or in other sustained release formulation. In some embodiments, the intratumorally delivered resiquimod or gardiquimod in the composition/formulation is given at the amount between 30 μg˜500 μg/tumor as free drug or given at the amount of 150 μg˜2.5 mg/tumor when it is encapsulated in micro/nano particle form or in other sustained release formulation. In some embodiments, the intratumorally delivered resiquimod or gardiquimod in the composition/formulation is given at the amount of 100 μg/tumor as free drug or given at the amount of 30 μg/tumor when it is encapsulated in micro/nano particle form or in other sustained release formulation.

In some embodiments, the intratumorally delivered 3M-052 or NKTR-262 in the composition/formulation of the current invention is given at the amount between 20 μg˜500 μg/tumor as free drug (free here means it is not in a sustained release form) or given at the amount of 50 μg 3 mg/tumor when it is encapsulated in micro/nano particle form or in other sustained release formulation. In some embodiments, the intratumorally delivered 3M-052 or NKTR-262 in the composition/formulation is given at the amount between 20 μg˜300 μg/tumor as free drug or given at the amount of 100 μg˜1 mg/tumor when it is encapsulated in micro/nano particle form or in other sustained release formulation. In some embodiments, the intratumorally delivered 3M-052 or NKTR-262 in the composition/formulation is given at the amount of 30 μg/tumor as free drug or given at the amount of 150 μg/tumor when it is encapsulated in micro/nano particle form or in other sustained release formulation.

In some embodiments, the intratumorally delivered poly IC (e.g. poly-ICLC) or CpG-ODN (e.g. SD-101 or ODN 1826 or ODN 2216) in the composition/formulation of the current invention is given at the amount between 1 mg˜10 mg/tumor as free drug (free here means it is not in a sustained release form) or given at the amount of 2˜20 mg/tumor when it is encapsulated in micro/nano particle form or in other sustained release formulation. In some embodiments, the intratumorally delivered poly IC or CpG-ODN in the composition/formulation is given at the amount between 1 mg˜5 mg/tumor as free drug or given at the amount of 2 mg˜10 mg/tumor when it is encapsulated in micro/nano particle form or in other sustained release formulation. In some embodiments, the intratumorally delivered poly IC or CpG-ODN in the composition/formulation is given at the amount of 2 mg/tumor as free drug or given at the amount of 5 mg/tumor when it is encapsulated in micro/nano particle form or in other sustained release formulation.

In some embodiments, the intratumorally delivered STING agonist (e.g. MK-1454 or ADU-S100) in the composition/formulation of the current invention is given at the amount between 100 μg˜1 mg/tumor as free drug (free here means it is not in a sustained release form) or given at the amount of 200 μg˜2 mg/tumor when it is encapsulated in micro/nano particle form or in other sustained release formulation. In some embodiments, the intratumorally delivered MK-1454 or ADU-S100 in the composition/formulation is given at the amount between 200 μg˜800 μg/tumor as free drug or given at the amount of 500 μg˜1 mg/tumor when it is encapsulated in micro/nano particle form or in other sustained release formulation. In some embodiments, the intratumorally delivered MK-1454 or ADU-S100 in the composition/formulation is given at the amount of 300 μg/tumor as free drug or given at the amount of 600 μg/tumor when it is encapsulated in micro/nano particle form or in other sustained release formulation.

The therapeutically effective amount of TLR agonist, STING agonist and other agent to treat cancer used in the current invention need to produce efficient anti-cancer activity (e.g. inhibiting tumor growth, inactivating cancer cells) while not producing unacceptable toxicity in the subject in need. The amount of TLR agonist, STING agonist and other agent can be 2-10 times higher when it is in sustained release form compared to the said free form (not in sustained release from). The dose can be determined experimentally by in vivo study including animal test and clinical study.

In some embodiments, the principle of cancer cell inactivating agent/agent that can enhance cancer cell antigen presenting in the current invention is to direct antibody or cytotoxic T cell to cancer cells, releasing tumor antigen for cancer immunotherapy. It will form in situ cancer vaccine and promote strong immune response with the locally injected immune activity enhancing agent. It has the general structure as following, which is also celled cell surface anchoring conjugate:

### Antibody Binding Molecule-Optional Linker-Cell Surface Anchoring Molecule Conjugate

In some embodiments, the cell surface anchoring molecule is cell membrane anchoring molecule, therefore the general structure of the conjugate is:

### Antibody Binding Molecule-Optional Linker-Cell Membrane Anchoring Molecule Conjugate

The antibody binding molecule can be the antigen of endogenous antibody in patient or the antigen of exogenous antibody given to the patient. Examples of exogenous antibody is the recombinant therapeutic antibody used for cancer treatment. The antigen can be the biopolymer (e.g. protein or its fragment) or peptide or small molecule used to induce/screen the antibody. It can be the epitope or mimotope of the antibody. Details and examples of them can be found in U.S. patent application Ser. Nos. 15/945,741, 16/271,877 and 16/924,184.

The antibody binding molecule can be affinity ligand for antibody other than antigen. It can be aptamer that can bind with antibody, antibody mimetic that can bind with antibody, a second antibody or antibody fragment that can bind with endogenous or exogenous antibody (e.g. a mouse antibody or Fab against human antibody's Fc region or human antibody's Fab region). Preferably the binding of the said ligand will not inhibit antibody's complement activation activity and/or inhibit antigen presenting effect induced by antibody binding.

In some embodiments, the conjugate comprises a mimotope therefore is called cell surface anchoring mimotope antigen conjugate. For example, the antibody binding molecule can also be the mimotope of Herceptin (trastuzumab). In another example, when the exogenous antibody is cetuximab, the antibody binding molecule can be epidermal growth factor receptor or its fragments or derivatives such as recombinant human EGF protein. The antibody binding molecule can also be the mimotope of cetuximab. Examples of the mimotope can be found in U.S. patent application Ser. Nos. 16/271,877 and 16/924,184. Similarly, other antibody drug including bispecific antibody, tri-specific antibody and antibody-drug conjugate targeting cancer cell and their antibody binding molecule (e.g. epitope or mimotope) can also be used.

U.S. patent application Ser. No. 15/945,741 by the current inventor disclosed native antigen-optional linker-cell surface anchoring molecule conjugate for cancer treatment. The native antigen in the disclosure and embodiments of said prior application can be replaced with affinity ligand such as antigen for the exogenous antibody given to the patient, which results in the antibody binding molecule-optional linker-cell surface anchoring molecule conjugate of the current invention, which are disclosed in U.S. patent application Ser. Nos. 16/271,877 and 16/924,184. The antigen for the exogenous antibody can be the biopolymer (e.g. protein or its fragment) or peptide or small molecule used to induce/screen the antibody. It can be the epitope or mimotope of the exogenous antibody.

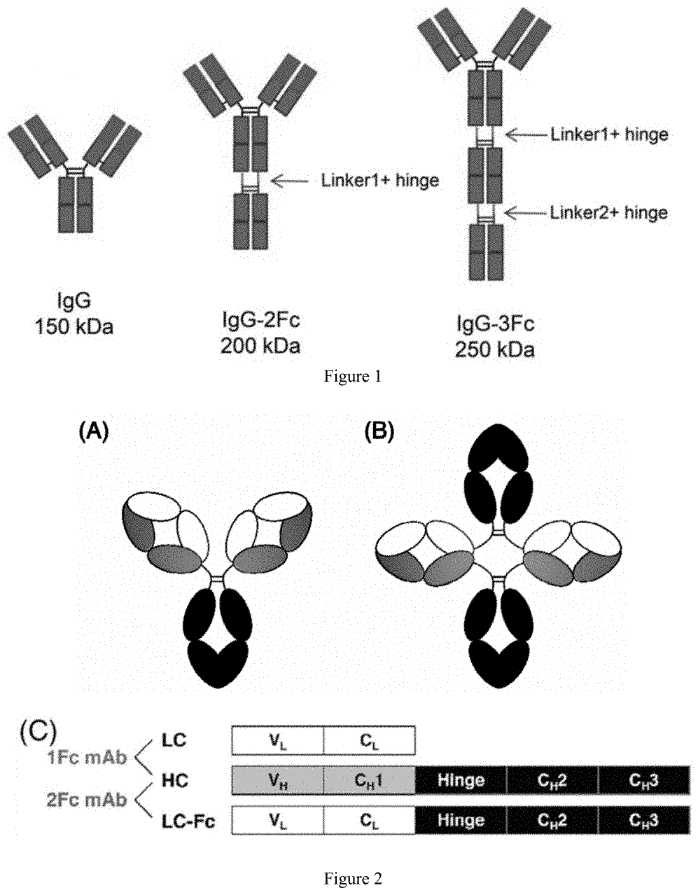

The example in FIG. 1 of U.S. patent application Ser. No. 16/271,877 shows the conjugate consisting of a 3β-cholesterylamine as cell surface anchoring molecule and Herceptin mimotope peptide and a short PEG as linker, to increase its potency. It can target none or low HER2 expression tumor. The immunity boosting agent can be co-injected intratumorally to turn the tumor into a in situ vaccine. FIG. 2 of U.S. application Ser. No. 16/271,877 illustrates the mechanism of an example of using antibody binding molecule-optional linker-cell surface anchoring molecule conjugate to increase the antigen presenting and cancer cell killing, wherein the cell surface anchoring molecule is a lipid type molecule that can insert into cancer cell membrane and the antibody binding molecule is Herceptin mimotope peptide and the exogenous antibody is Herceptin.

In some embodiments, the preferred cell membrane anchoring molecule for the conjugate is fatty acid or long alkyl chain or 33-cholesterylamine or its analogues or derivatives, 3β-cholesterylamine type molecule enables endosome recycling of conjugate for long cell surface anchoring half-life. It can be either in monomer or dimer or trimer or oligomer format within the conjugate. The antibody binding molecule can also be either in monomer or dimer or trimer or oligomer format within the conjugate. Examples of 3β-cholesterylamine, 3β-cholesterylamine containing moiety and their derivatives that can be used for the conjugate can be found in U.S. patent application Ser. No. 15/945,741. Exemplary structures of the conjugate include Herceptin mimotope-cholesterylamine, cetuximab mimotope-cholesterylamine, Herceptin mimotope-linker-cholesterylamine, cetuximab mimotope-linker-cholesterylamine, cetuximab mimotope oligomer-linker(optional)-cholesterylamine, Herceptin mimotope oligomer-linker(optional)-cholesterylamine, Herceptin mimotope-linker-cholesterylamine-cetuximab mimotope as disclosed in U.S. patent application Ser. Nos. 16/271,877 and 16/924,184.

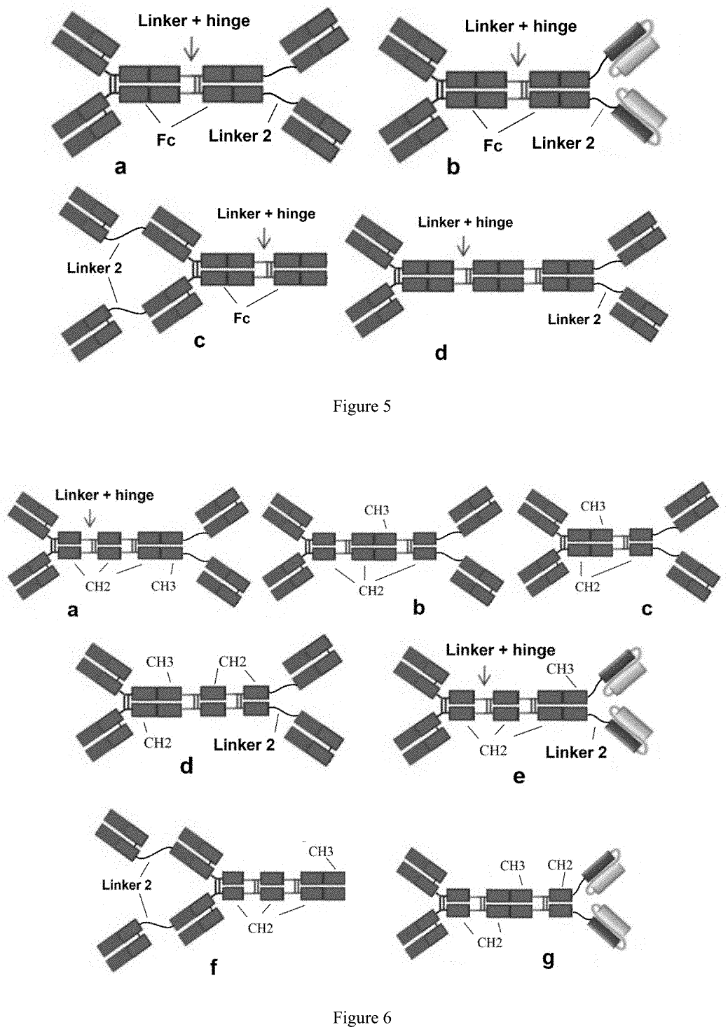

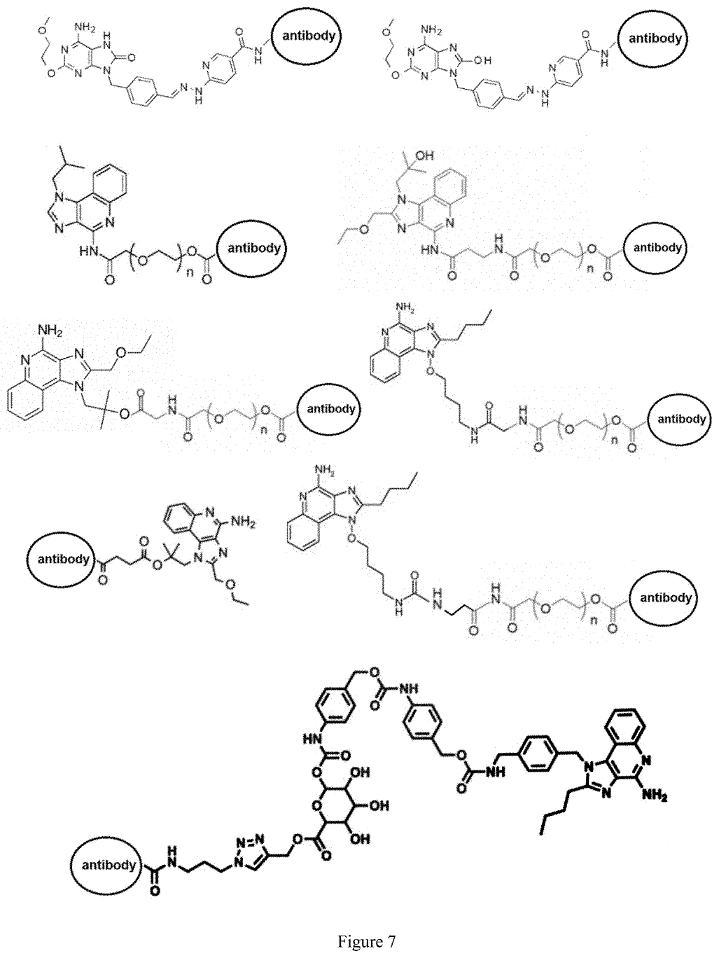

More than one unit of antigen (e.g. mimotope or native antigen of U.S. patent application Ser. Nos. 15/945,741 and 16/271,877) or affinity ligand for antibody, more than one type of antigen (e.g. mimotope or native antigen) or affinity ligand for antibody and more than one unit of cell surface anchoring molecule such as cholesterylamine can be incorporated in the conjugate as shown in FIG. 4 of U.S. patent application Ser. No. 16/271,877. One or more copied of immune activity enhancing agent such as TLR agonist or STING agonist can also be conjugated to the backbone. They can also be conjugated to a soluble polymer backbone (e.g. dextran, poly peptide, poly acrylic acid or the like) such as those shown in FIG. 5 of U.S. patent application Ser. No. 16/271,877. Insoluble polymer back bone can also be used, which is essentially a nano or micro particle. The cell membrane/surface anchoring molecule can also be molecule other than cholesterylamine, such as lipid molecule and cell membrane anchoring peptide as described in U.S. patent application Ser. Nos. 15/945,741, 16/271,877 and 16/924,184. In one example, Herceptin mimotope-membrane anchoring peptide conjugate has the structure shown in FIG. 7 of U.S. patent application Ser. No. 16/271,877.

The cell surface anchoring molecule in the antibody binding molecule-optional linker-cell surface anchoring molecule conjugate can also be reactive molecule/functional group that can covalent attach to cell surface molecules such as membrane proteins by chemical reaction once in contact, it has the general structure of antibody binding molecule-optional linker-cell surface reactive moiety conjugate. Examples of them can be found in disclosure of U.S. patent application Ser. Nos. 16/271,877 and 16/924,184. Examples of Herceptin mimotope peptide conjugate containing an NHS ester are shown in U.S. patent application Ser. No. 16/271,877.

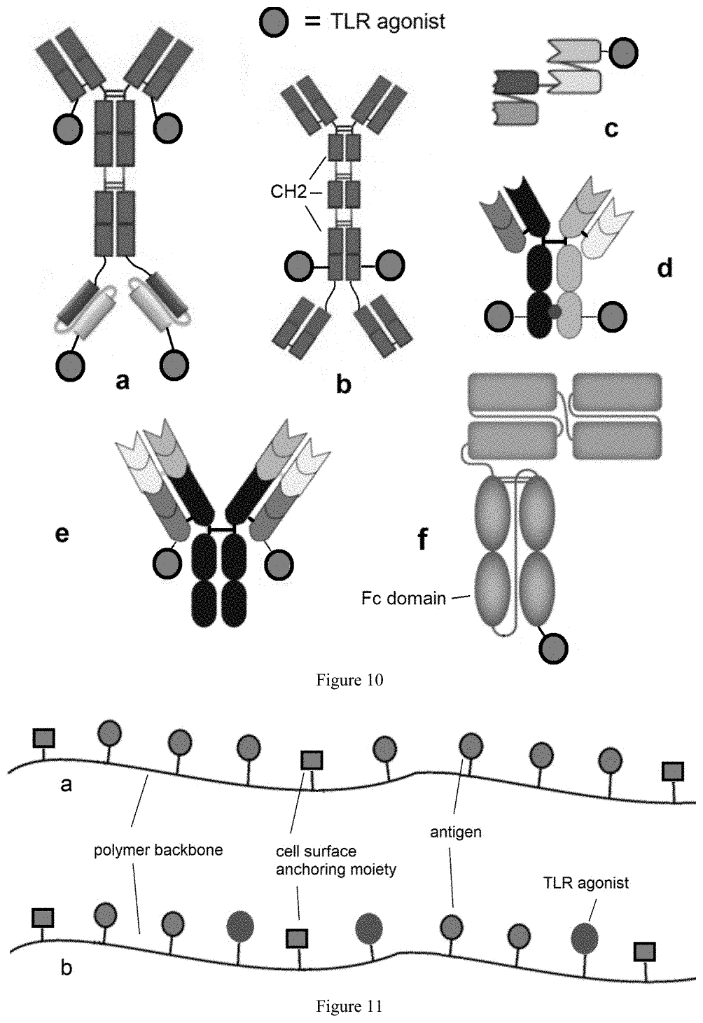

The conjugate can further comprise a cancer cell binding domain to increase its targeting to cancer cell, which will also allow intravenous (iv) or IM or SC injection instated of intratumoral injection. Therefore the antibody binding molecule-optional linker-cell surface anchoring molecule conjugate has the structure of antibody binding molecule-optional linker-affinity ligand for cancer cell surface molecule conjugate, with optional cell membrane inserting lipid like molecule such as that shown in FIG. 10 of U.S. patent application Ser. No. 16/271,877. It can also be simply a Fc fused affinity ligand for cancer cell surface molecule, such as Fc-Anticalin against cell surface molecule, FcMBL (Fc fused mannose binding lectin) that can bind with cancer cell. The affinity ligand can be not specific to cancer cell surface marker if it is injected intratumorally as local injection will generate enough local binding. It can be the ligand for none cancer specific cell surface molecule such as EpCAM. Preferably the antibody or antibody mimetic or conjugate used in the current invention has long cell surface half-life. Additional examples are disclosed in U.S. patent application Ser. Nos. 16/271,877 and 16/924,184.

The said antibody binding molecule-optional linker-cell surface anchoring molecule conjugate is to introduce Fc onto cancer cell surface upon intratumoral injection, which will kill the cancer cell and enhance tumor antigen presenting by ADCC, complement activation and Fc mediated phagocytosis to enhance APC. An alternative method and agent to attach antibody Fc domain to cancer cell surface is to use Fc (or its fragment)-optional linker-cell surface anchoring molecule conjugate instead, which are disclosed in U.S. patent application Ser. Nos. 16/271,877 and 16/924,184. Once being injected intratumorally, preferably in combination with a vaccine adjuvant type agent as described above, it will turn the tumor into an in situ cancer vaccine. They are essentially the conjugate by replacing the antibody binding molecule of above described antibody binding molecule-optional linker-cell surface anchoring molecule conjugate with Fc or its fragment. Example is shown in FIG. 11 of U.S. patent application Ser. No. 16/271,877.

Another agent that can be injected to the tumor to treat cancer is sialidase or sialidase conjugated with cholesterylamine or lipid type molecule as described in U.S. patent application Ser. Nos. 15/945,741, 16/271,877 and 16/924,184. The sialidase can be either bacterial sialidase or viral sialidase or animal sialidase or human sialidase in therapeutical effective amount (e.g. 0.01˜5 mg per injection). Preferably it is injected together with the cancer cell inactivating agent into the tumor at therapeutical effective amount. It can be co-formulated with the vaccine adjuvant type agent.

The cancer cell inactivating agent is not limited to antigen-optional linker-cell membrane anchoring molecule conjugate. It can be any agent that can lyse the cancer cell when intratumoural injected as disclosed in U.S. patent application Ser. Nos. 15/945,741, 16/271,877 and 16/924,184. For example, they can be acid or base, organic solvent, perforin, C3b, C5b, membrane attack complex, cell inactivating peptide and antibiotics, and cell inactivating detergent/surfactant. For example, they can be used as injection at the concentration between 0.01˜100 mg/mL or other concentration that can kill the cancer cells.

The current invention discloses novel compositions/formulations to treat tumor cell and cancer. The formulation comprises one or more said cancer cell inactivating agent and/or agent can enhance cancer cell antigen presenting (antigen presenting booster) and immune activity enhancing agent in a pharmaceutical acceptable carrier. It can be injectable solution or solid dosage form such as lyophilized formulation that can be reconstituted to injectable solution. The formulation contains cancer cell inactivating agent/antigen presenting booster and immune activity enhancing agent as well as pharmaceutical acceptable excipients suitable for injection. They can be in form of active drug, prodrug, liposome, micelle, emulsion, gel formulation, implant, thermal phase changing formulation, insoluble precipitate (e.g. in complex with condensing agent), conjugated to polymer drug carrier (e.g. dextran) or coated on or encapsulated in biodegradable micro particle/nano particle. Suitable size of the particle is between 10 nm˜100 μm.

Diluent or carriers employed in the compositions can be selected so that they do not diminish the desired effects of the composition. Examples of suitable compositions include aqueous solutions, for example, a saline solution, 5% glucose. Other well-known pharmaceutically acceptable liquid carriers such as alcohols, glycols, esters and amides, may be employed. In certain embodiments, the composition further comprises one or more excipients, such as, but not limited to ionic strength modifying agents, solubility enhancing agents, sugars such as mannitol or sorbitol, pH buffering agent, surfactants, stabilizing polymer, preservatives, and/or co-solvents. In certain embodiments, a polymer matrix or polymeric material is employed as a pharmaceutically acceptable carrier. The polymeric material described herein may comprise natural or unnatural polymers, for example, such as sugars, peptides, protein, laminin, collagen, hyaluronic acid, ionic and non-ionic water soluble polymers; acrylic acid polymers; hydrophilic polymers such as polyethylene oxides, polyoxyethylene-polyoxypropylene copolymers, and polyvinylalcohol; cellulosic polymers and cellulosic polymer derivatives such as hydroxypropyl cellulose, hydroxyethyl cellulose, hydroxypropyl methylcellulose, hydroxypropyl methylcellulose phthalate, methyl cellulose, carboxymethyl cellulose, and etherified cellulose; poly(lactic acid), poly(glycolic acid), copolymers of lactic and glycolic acids, or other polymeric agents both natural and synthetic. In certain embodiments, compositions provided herein may be formulated as films, gels, foams, or and other dosage forms. Suitable ionic strength modifying agents include, for example, glycerin, propylene glycol, mannitol, glucose, dextrose, sorbitol, sodium chloride, potassium chloride, and other electrolytes. In certain embodiments, the solubility of the cell surface anchoring antigen conjugates may need to be enhanced. In such cases, the solubility may be increased by the use of appropriate formulation techniques, such as the incorporation of solubility-enhancing compositions such as mannitol, ethanol, glycerin, polyethylene glycols, propylene glycol, poloxomers, and others known in the art. Surfactants can be employed in the composition to deliver higher concentrations of cell surface anchoring antigen conjugates and immune function enhancing agents. The surfactants function to solubilize the insoluble and stabilize colloid dispersion, such as micellar solution, microemulsion, emulsion and suspension. Suitable surfactants comprise polysorbate, poloxamer, polyoxyl 40 stearate, polyoxyl castor oil, tyloxapol, triton, and sorbitan monolaurate. In some embodiments, preservatives are added to the composition to prevent microbial contamination during use. Suitable pH buffering agents for use in the compositions herein include those disclosed in U.S. patent application Ser. Nos. 15/945,741, 16/271,877 and 16/924,184.

In some embodiments, separate or sequential administration of the composition and other agent is necessary to facilitate delivery of the composition into the patient. In certain embodiments, the composition and the other agent can be administered at different dosing frequencies or intervals. For example, one composition can be administered daily or weekly, while the other agent can be administered less frequently. Additionally, as will be apparent to those skilled in the art, the composition and the other agent can be administered using the same route of administration or different routes of administration.

In making pharmaceutical compositions that include cell surface anchoring conjugates described herein, the active ingredient is usually diluted by an excipient or carrier and/or enclosed within such a carrier that can be in the form of a capsule, sachet, paper or other container. When the excipient serves as a diluent, it can be a solid, semi-solid, or liquid material (as above), which acts as a vehicle, carrier or medium for the active ingredient. Thus, the compositions can be in the form of films, gels, powders, suspensions, emulsions, solutions, containing, for example, up to 50% by weight of the active compounds, sterile injectable solutions, and sterile packaged powders. Some examples of suitable excipients include lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, sterile water, syrup, and methyl cellulose. The formulations can additionally include: wetting agents; emulsifying and suspending agents; and preserving agents such as methyl- and propylhydroxy-benzoates. Liquid solution as used herein refers to solutions, suspensions, emulsions, ointments, sprays, liposomes which are well known in the art.

In certain embodiments, the cell surface anchoring conjugates or a composition comprising the same, is lyophilized prior to, during, or after, formulation. In certain embodiments, the cell surface anchoring conjugates, or a composition comprising the same, is lyophilized in a pharmaceutical formulation comprising a bulking agent, a lyoprotectant, or a mixture thereof. In certain embodiments, the lyoprotectant is sucrose or mannitol. In certain embodiments, the cell surface anchoring conjugates, or a composition comprising the same, is lyophilized in a pharmaceutical formulation comprising mannitol and sucrose. Exemplary pharmaceutical formulations may comprise about 1-20% mannitol and about 1-20% sucrose. The pharmaceutical formulations may further comprise one or more buffers, including but not limited to, phosphate buffers. Accordingly, also provided herein is a lyophilized composition comprising a drug conjugate, nanoparticle or composition comprising the same as described herein.

The composition and formulation of the current invention can be in a gel form or high viscosity liquid. Gels are used herein refer to a solid, jelly-like material that can have properties ranging from soft and weak to hard and tough. As is well known in the art, a gel is a non-fluid colloidal network or polymer network that is expanded throughout its whole volume by a fluid. A hydrogel is a type of gel which comprises a network of polymer chains that are hydrophilic, sometimes found as a colloidal gel in which water is the dispersion medium. Hydrogels are highly absorbent and can contain a high degree of water, such as, for example greater than 70% water. In some embodiments, the gel described herein comprises a natural or synthetic polymeric network. In some embodiments, the gel comprises a hydrophilic polymer matrix. In other embodiments, the gel comprises a hydrophobic polymer matrix. In certain embodiments, the gel is biocompatible and absorbable. In certain embodiments, the gel is formed after being administered to the patient. In certain embodiments, the gel is administered to the patient prior to, during or after surgical intervention.

The composition and formulation can contain viscosity enhancing agent to increase its viscosity, which acts as a sustained release formulation. In certain embodiments, the formulation is a viscous liquid. In certain embodiments, the injection has a viscosity greater than 10,000 cps at room temperature. In certain embodiments, the injection has a viscosity greater than 100,000 cps at room temperature. In certain embodiments, the injection has a viscosity greater than 1,000,000 cps at room temperature. In certain embodiments, the injection has a viscosity of 10,000,000 cps at room temperature. Example of the viscosity enhancing agent can be found readily from known pharmaceutical acceptable excipients such as hyaluronic acid (linear or cross-linked form), HPMC, MC, CMC, starch and carbomer. In some embodiments, the viscosity enhancing agent is biodegradable.

In some preferred embodiments, the composition of the current invention is in a sustained release system to release the active drug (e.g. TLR agonist, antibody, antigen conjugate) within in an extended period of time, e.g. 50% drug released in several days to several weeks. The formulation is an extended (sustained) release formulation. In some preferred embodiments, the composition of the current invention is within a in situ gelling system and the formulation is said drug loaded in situ gelling formulation. In situ gelling systems are often polymeric formulations that are in sol (solution) forms before entering in the body, but change to gel forms under the physiological conditions. The sol-gel transition depends on one or a combination of different stimuli, like pH change, temperature modulation, solvent exchange, ultra violet irradiation and the presence of specific ions or molecules. Drug delivery systems having such properties can be widely used for sustained delivery vehicle preparation of the bioactive molecules. Some important advantages of these smart systems are ease of application and reduced frequency of administration, as well as protection of drug from environmental condition changes. Various natural and synthetic polymers undergo in situ gel forming and potentially can be used. Pectin, xyloglucan, gellan gum, chitosan and alginic acid are some of the natural polymers. The pectin gelation occurs in the presence of calcium ions. Xyloglucan exhibits thermally reversible gelation with body temperature. Dilute aqueous solutions of alginates form firm gels, on addition of di and trivalent metal ions, such as the Ca′ in body fluid. Examples of alginate can be used include sodium alginate, potassium alginate, ammonium alginate and other pharmaceutically acceptable amine salt of alginate. For example, sodium alginate and hydroxypropyl methyl cellulose can be used in the in situ gelling formulation. In situ gel formation of gellan gum occurs due to temperature modulations or the cations induced. Temperature and ionic condition (e.g. Ca′) in body fluid cause a transition of aqueous solution of gellan into the gel state. Carbopol (poly acrylic acid) is a well-known pH dependent polymer, which stays in solution form at acidic pH but forms a low viscosity gel at alkaline pH. An in situ gel can be formulated using carbopol and hydroxypropyl methylcellulose (HPMC). The latter is used to impart the viscosity to the carbopol solution, while reducing its acidity. Aqueous solution of carbopol-HPMC system is also an in situ gelling system. Pluronic F-127 is a triblock copolymer with nonionic nature, which undergoes in situ gelation by temperature change. It can be used together with Carbopol 934 and HPMC to prepare in situ gel. Chitosan aqueous solution forms a hydrated gel, like precipitate, at pH exceeding 6.2. Adding polyol salts, bearing a single anionic head, like glucose phosphate salts to chitosan aqueous solution can transform the cationic polysaccharides solution into thermally sensitive pH dependent gel. The sol form of such formulation (at the room temperature) turns into gel implants, when injected in vivo. Examples of them can be found in PMID: 25237648 and can be readily adopted for the current invention. In some embodiments, the gel is made of hyaluronic gel with optional calcium salt or ferric salt, for example the calcium ions and hyaluronic gel material is characterized in that: comprise hyaluronic acid, CaCl₂) or FeCl₃, and deionized water at weight ratio 0.01˜10:0.01˜10:100.

Examples of the in-situ gelling polymers used in in situ gelling system include chitosan, alginic acid, xyloglucan, gellan gum, sodium hyaluronate, pectin, hydroxypropyl methylcellulose (HPMC), methylcellulose (MC), carboxymethylcellulose, cellulose acetate phthalate (CAP), PGA, prolifeprospan, Carbopol, poloxamer such as Pluronics, poly(lactide-co-glycolide) (PLGA), poly(D,L-lactide-co-hydroxymethyl glycolide) (PLHMGA).

The drug loaded in situ gelling system can use pH triggered in situ gelling polymers: pH triggered in situ gelling systems are solutions, which upon exposure to the pH of the body fluid converts into the gel phase e.g. such as carboxymethylcellulose, hyaluronate, cellulose acetate phthalate and Carbopol. Cellulose acetate phthalate latex remains free flowing solution at acidic pH (˜pH 4) and transform into the gel at neutral pH (pH7). Polyacrylic acid commercially known as Carbopol is a widely used polymer undergoes sol to gel transition in aqueous solution as the pH is raised above its pKa of about 5.5. the formulation of these type of system can have a low pH (4-5) to remain solution and become gel once inside body due to the pH change. Polyacrylic acid (e.g. Carbopol® 934) can be used as the gelling agent with HPMC (Methocel K4M) as viscosity enhancer. Polyacrylic acid (Carbopol) can be used as the gelling agent in combination with chitosan (as viscosity enhancer). The 0.4% w/v Carbopol/0.5% w/v chitosan based in situ gelling system is in liquid state at room temperature and at the pH of formulation i.e. pH 6.0, and underwent rapid transition into the viscous gel phase at pH 7.4 inside body.

The drug loaded in situ gelling system can use temperature triggered in situ gelling polymers: temperature triggered in situ gelling polymers remains liquid at low temperature (below 20° C.) and undergoes gelation at physiological temperature (35-37° C.). Following are some examples of temperature triggered in situ gelling polymeric systems: Poloxamers: Poloxamers, commercially known as Pluronic®, are the thermoreversible polymers commonly used for formation of thermosensitive in situ gelling systems. Upon heating from 4° C. to 23° C. or more, aqueous solution of Pluronic F127 or Poloxamer 407 at a concentration of ≥15%, transformed to a semisolid gel from a low viscosity solution. For example, the system can contain 20% w/w Poloxamer 407 and 10% w/w Poloxamer P188. A low viscosity aqueous solution of Poloxamer 407 (P407), at a concentration of ≥18% w/w (a 7:3 ratio of PEO and PPO) can be converted to a gel under the ambient temperature and the addition of hyaluronic acid (HA) in the Poloxamers blends can delay the gelation temperature by few degree Celsius and at specific concentration of Poloxamer/HA it is possible to get a thermoreversible gel with a gelation temperature close to body temperature. Viscosity enhancing agents (HPMC, MC and CMCNa) can be added to the 15% w/w PF-127 to form hydrogel, for example 15% PF-127 formulations containing 3% methylcellulose can be used as a temperature triggered in situ gelling system to load drug.

Poloxamines is another temperature triggered in situ gelling system, commonly known as Tetronics (tetra functional block copolymers of ethylene and propylene oxide), e.g. tetronic-oligolactide copolymer (made of Tetronic®1307 and pure L-lactide).

Another temperature triggered in situ gelling system is cellulose derivatives: ethyl (hydroxyl ethyl) cellulose, methylcellulose and HPMC are some of the cellulose derivatives which are being used as in situ gelling polymers. Aqueous solutions of ethyl (hydroxyethyl) cellulose (EHEC) exhibit thermosensitive gelation. On addition of sodium dodecyl sulphate or cetyl triammonium bromide, EHEC (1%-4% w/w) solutions undergoes sol-to-gel phase transition upon heating to 30-40° C. and forms stiff and clear gels. Some cellulose derivatives remain liquid at low temperature and become gel upon heating, for example aqueous solutions of methylcellulose and HPMC undergoes phase transition into gels between 40-50° C. and 75-90° C. respectively. However, phase transition temperatures of methylcellulose and HPMC are higher than the physiological temperatures, but can be lowered by making chemical or physical changes in the polymers. For example, addition of NaCl in methylcellulose or lowering the hydroxypropyl molar substitution of HPMC, the phase transition temperatures can be reduced to 32-34° C. and 40° C., respectively in these polymers.

The gelation temperature of 1% methylcellulose solution is decreased to the physiological temperature i.e. 37° C. by addition of fructose and sodium citrate tribasic dihydrate (SC) in different proportions. 1 to 5% SC can be added in the methylcellulose (1%) and fructose (10%) as the temperature triggered in situ gelling system.

Xyloglucan, a polysaccharide obtained from tamarind seed and approved for use as food additive. Partially degraded xyloglucan by β-galactosidase to >35% galactose removal ratio exhibits thermally reversible gelation in dilute aqueous solutions. The sol-gel transition temperature of xyloglucan varies with degree of galactose elimination and polymer concentration and related inversely, for example, on increasing the galactose removal ratio from 35 to 58% the sol-gel transition of xyloglucan was observed to be decreased from 40 to 5° C. Xyloglucan forms gels by the lateral stacking of rod like chains. The 1.5% w/w xyloglucan based in situ gelling formulation showed similar miotic response as shown by 25% w/w Pluronic F127 gel. The thermoreversible phase transition temperature of poly (N-isopropylacrylamide) (PNIPAAm), a well-known thermosensitive polymer is 32° C. Because of its phase transition temperature closer to human body surface temperature, this in situ gel forming polymer can be utilized.

Addition of methylcellulose, HPMC, CMC, mannitol and sorbitol as viscosity enhancing agents to in situ gelling polymer can be utilized. Thermally sensitive neutral solutions based on chitosan/polyol salt combinations as shown in publication of PMID: 10985488 is also a temperature triggered in situ gelling system that can be used.

The drug loaded in situ gelling system can use ion triggered in situ gelling polymers. These include polymers whose solution viscosity increases upon exposure to ionic concentration of the body fluids such as tear fluids. It is also called osmotically induced gelation. Ion sensitive polymers can crosslink with cations (monovalent, divalant) present in lacrimal fluid on ocular surface and enhance the retention time of drug. Ion triggered in situ gelling polymeric systems include gellan gum which is commercially known as Gelrite®, and alginic acid/sodium alginate: Sodium alginate is a natural hydrophilic polysaccharide approved by FDA for human use as wound dressing material and as food additives consist of (1→4) linked β-D-mannuronic acid (M) and α-L guluronic acid (G) units of varying composition and sequence. Alginate transforms into stable gel upon exposure to divalent cations such as Ca²⁺ in the body. The % of guluronic acid in polymer backbone plays a major role in alginate gelation and drug release. Alginates with guluronic acid contents >65% gelled instantaneously, whereas with low guluronic acid contents gelled slowly and forms weak gels. Ion activated in situ gelation of sodium alginate in combination of other viscosity enhancer such as HPMC can be used. Low contraction of Ca salt can be preloaded into the formulation before injection, which will not cause gelling in vitro but will to help the gelling in vivo, e.g. 0.1%˜0.6% calcium gluconate solution. The amount of Ca salt to be added to help gelling in vivo but not cause gelling in vitro is dependent of the concentration of alginate in the formulation. Higher concentration of alginate requires lower concentration of Ca salt and low concentration of alginate can tolerate higher concentration of Ca salt while maintain the non-gel state in vitro. The suitable amount of Ca salt to be preloaded into the formulation can be determined experimentally easily by adding different amount of Ca salt to the alginate containing formulation and select the highest amount of Ca salt that does not produce un injectable gel in vitro. In some examples, the in situ gelling system matrix is 1% w/v sodium alginate (e.g. VLVG, NovaMatrix, FMC Biopolymers, Drammen, Norway) and 0.3% w/v calcium D-gluconate in the final drug containing formulation of the current invention.

Combination of gelling enhancing agent including polymers having different gelation mechanisms can also be used. To reduce the amount of polymers required for gelation and to get better gels with improved gelling properties combination of two or more polymers with different gelation mechanism can be used for developing in situ drug delivery system. For example, a combination of thermosensitive polymers, methylcellulose or HPMC and pH triggered polymer Carbopol can be used. The former polymers exhibited thermal gelation and the latter pH dependent gelation. The final formulation formed an easy flowing formulation, which reversibly gelled with a sol-gel transition between 25° C. and 37° C. as well as with a pH increase from 4.0 to 7.4. In some examples, 25% (w/v) Pluronics and 30% (w/v) CAP are used. In one example poloxamer+chitosan based in situ gelling system can be used. Poloxamer-chitosan (16:1) system showed optimum gelation temperature 32° C. In one example a combination of pH and ion triggered polymers based in situ gelling systems can be prepared by blending three different polymers namely Carbopol 940, sodium alginate and guar gum. In one example a formulation can consist of 15% Pluronic F127 and 0.1% low molecular weight chitosan. 0.3% and 14% (w/w) concentrations of Carbopol and Pluronic can be used for preparation of in situ gelling formulations. In another example Poloxamer 407 and 188 are used as thermosensitive polymers and Carbopol 1342P NF is used as pH sensitive polymer and the combined solutions formed gels under physiological conditions. In one example ˜15% Pluronic F127 combined it with polymers like HPMC as a viscosity increasing agent or with polymers such as Carbopol 940, xanthan gum, and sodium alginate (high glucuronic acid content) for pH and cation-triggered sol-gel transition can be used. The combination of methylcellulose or HPMC and Carbopol in some examples. In one example concentration of sodium alginate solution for the in situ gelation is 2% w/w and that for Pluronic F127 it is 14% (w/w). In some examples Triblock (TB) polycaprolactone-polyethylene glycol-polycaprolactone [(PCL-PEG-PCL), BAB] and pentablock copolymers (PBCs) polylactic acid (PLA) [(PLA-PCL-PEG-PCL-PLA), CBABC] and [(PEG-PCL-PLA-PCL-PEG), ABCBA] can be used. In one example in situ gelling system is sodium alginate as ion sensitive polymer and methylcellulose as viscosity enhancing agent. In some examples, Polyacrylic acid (Carbopol 940) or hyaluronic acid, Pluronic F127 and gellan gum are used for pH-triggered in situ gelation, thermoreversible gelation and ion activated system, respectively. HPMC is added with Carbopol or hyaluronic acid as viscosity enhancer and in combination of Pluronic F127 for reducing the concentration of Pluronic F127. Gelrite® is used for cation induced gelation (0.6%). In some embodiments, drug loaded thermosensitive PEG-PCL-PEG (PECE) hydrogel by synthesizing PECE block polymers by coupling MPEG-PCL copolymer using IPDI reagent having sol-gel transition as a function of temperature can be used. The formulation containing PECE (30% w/v) aqueous solution exhibited sol-gel transition at 35° C.

Furthermore, drug loaded liposome, emulations including nanoemulsions, suspension, cyclodextrin, micelles, nanoparticles or microparticles can also be incorporated within the in situ gel.

Furthermore, drug loaded liposome, emulations including nanoemulsion, suspension, cyclodextrin, micelles, nanoparticles or microparticles can also be incorporated within the in-situ gel. The drug loaded in situ gelling system can use reactive in situ gel as well, which forms hydrogel by crosslinking after mixing two reactive components together. In some embodiments, hydrogel is prepared by simple mixing of glycol chitosan and oxidized alginate aqueous solution, which can be injected right after being mixed together when it is still injectable as complete crosslinking reaction takes time. The polymer (e.g. hyaluronic acid) and crosslinking agent (e.g. H₂O₂, pentasodium tripolyphosphate) can also be co injected (e.g. using a dual syringe type device) to the body to allow crosslinking take place in vivo. In some embodiments, PEG hydrogel is prepared through thiol-maleimide reaction utilizing 4 arms PEG-Mal and 4 arm PEG-SH. In some embodiments, in situ gelling drug delivery system is thiolated poly (aspartic acid) (ThioPASP). In some embodiments, hydrogel is composed of maleimide-modified c-polyglutamic acid (c-PGA-MA) and thiol end-functionalized 4-arm poly (ethylene glycol) (4-arm PEG-SH) such as those in Acta Biomaterialia 86 (2019) 280-290.

Another type of reactive situ gelling system matrix is injectable drug eluting elastomeric polymer (iDEEP), such as those described in publication of PMID: 22301346. For example, poly (ethylene glycol maleate citrate), PEGMC, is dissolved in deionized water (20 wt %), and combined with poly (ethylene glycol diacrylate) (12 wt %), and tetramethylethylenediamine (0.5 wt %) as iDEEP Part A, suitable amount of drug is also loaded in iDEEP Part A. The iDEEP Part B Component (iDEEP-B) is prepared by dissolving ammonium persulfate redox initiator (0.25 wt %) in deionized water. Combining the Part A and B solutions in a 2:1 ratio, respectively, produces iDEEP gels.

Photocrosslinkable agent can also be used to form in situ gel, which is also a reactive matrix and the gelling reaction is triggered by light irradiation. Examples of photocrosslinkable include polyethylene glycol diacrylate (PEGDA) and photocrosslinkable chitosan hydrogel, such as those described in publication of PMID: 19160155. PEGDA gels rapidly at room temperature in the presence of a photoinitiator and light (e.g. UV light).

In some embodiments, drug loaded in situ gelling implant/insert can be used. For example, carboxymethylcellulose sodium (CMC) and sodium alginate (ALG) combination can be used as the matrix. In some embodiments, the drug loaded in situ gelling is in chitosan/HPMC based polymer matrix. In some embodiments, the drug loaded injectable gel or nano/micro particles is in biochronomer (tri(ethylene glycol) poly(orthoester), TEG-POE) based polymer matrix. For example, the injectable gel is 80% TEG-POE (MW 6 kDa), ˜19% methoxypoly(ethylene glycol) (MW 550 Da) and 0.1-1% (by weight) drug.

In some embodiments, the drug loaded in situ gelling is in chitosan-calcium alginate gel microsphere based polymer matrix, such as those described in patent number CN1628861A. For example, the matrix can be chitosan-calcium alginate gel microsphere type material, which is composed of calcium alginate gel microspheres optionally covered with chitosan in 0.5-4.0% sodium alginate solution. The particle size of the calcium alginate gel microspheres is between 1-200 μm; the ratio of the chitosan-calcium alginate gel microspheres to the sodium alginate solution is 10:1-10:30 by volume. The drug can be either encapsulated in the microsphere or in the alginate solution phase or both.

Another in situ gelling material can be used in the said formulation is biodegradable water insoluble polymer such as poly(D,L-lactide-co-hydroxymethyl glycolide) (PLHMGA), PLA, PLGA, PCL, PGA, prolifeprospan such as prolifeprospan 20 or PHB. It can be dissolved in biocompatible water miscible organic solvent such as N-methyl pyrrolidone (NMP) or DMSO as matrix to load the drug, the drug can be dissolved/dispersed in the PGA or PLGA solution (e.g. 10%˜50% PLGA in N-methyl pyrrolidone) or two components are combined immediately before injection. In some embodiments, 50:50 lactide/glycolide PLGA or PLGA with lower lactide content can be used, e.g. 10:90 lactide/glycolide PLGA. When this formulation is injected into the body the water miscible organic solvent dissipates and water penetrates into the organic phase. This leads to phase separation and precipitation of the polymer forming a depot at the site of injection as sustained release implant type material. Although it is not a classic hydro gel gelling system, it is still called gelling in the current invention for illustration purpose. Examples can be found in Atrigel™ delivery system and those in publication of PMID: 24929039.

Other gelling or high viscosity materials that can be used in the current invention include: RAD16 peptide, collagen, PNIPAAm-g-MC, the polymer in patent number CN102344559A, modified hyaluronic acid sodium gel in patent number CN104086788B, injectable hyaluronic acid/polyethylene glycol hydrogel in patent number CN106519072A, sodium hyaluronate collagen hydrogels in patent number CN107189119A, Pluronic® F127 and Pluronic® F68, PNIPAAm, poly(lactic acid-co-glycolic acid)-poly(ethylene glycol)-poly(lactic acid-co-glycolic acid) (PLGA-PEG-PLGA) hydrogel (e.g. those in International Journal of Pharmaceutics 490 (2015) 375-383), thermosensitive triblock polymer poly-(DL-lactic acidco-glycolic acid) (PLGA)-polyethylene glycol (PEG)-PLGA (e.g. those in publication of PMID: 20334543), system containing poloxamer 188/poloxamer 407/carbopol 934/HPMC (e.g. those in publication of PMID: 24790559), injectable bioresponsive gel depot (e.g. those in publication of PMID: 29786888), PVA-TSPBA hydrogels (e.g. those in publication of PMID: 29467299), fibrin hydrogel (e.g. those in patent number CN110393699A), thermo gelling polyurethane/PEG block copolymer (e.g. the amine-functionalized ABA block copolymer, poly(ethylene glycol)-poly(serinol hexamethylene urethane), consists of a hydrophobic block (B): poly(serinol hexamethylene urethane) and a hydrophilic block (A): poly(ethylene glycol), e.g. those disclosed in publication of PMID: 20937526); injectable self healing polymer nanoparticle (PNP) hydrogel HPMC-C12 (dodecyl modified hydroxypropylmethylcellulose) combined with poly(ethylene glycol)-b-poly(lactic acid) (PEG-PLA) nanoparticles (NPs), 2 wt % HPMC-C12+10 wt % NP as those in publication of PMID: 33145416); vaccine self-assembling immune matrix made of (RADA)4 synthetic oligopeptide (e.g. those described in publication of PMID: 25609075); thermal-sensitive hydrogel formulated with N-[(2-hydroxy-3-trimethylammonium) propyl] chitosan chloride (HTCC) and α,β-glycerophosphate (α,β-GP) (e.g. those in publication of PMID: 22192540); poly(d, 1-lactide)-poly(ethylene glycol)-poly(d,l-lactide) (PDLLA-PEG-PDLLA,PLEL) (e.g. those described in doi.org/10.1016/j.apmt.2020.100608); the gelling system in patent application number WO2014006215A1; injectable PEG-b-poly(L-alanine) hydrogel (e.g. those in publication of PMID: 31149045); injectable chitosan-alginate porous gel in publication of PMID: 30444317; and also the agent that has low viscosity at high shear rate (e.g. 100S⁻¹ reminiscent of the injection process) and high viscosity (preferably >10 times higher) at a low shear rate (e.g. the condition after being injected).

Other agent that has low viscosity at high shear rate and high viscosity at a low shear rate can also be used as matrix in the formulation of the current inventions either alone or together with other in situ gelling matrix. Example of them include materials having exhibiting pseudoplastic viscosity such as those polysaccharide disclosed in WO2013077357A1, such as xanthan gum, carrageenan, gellan gum, guar gum, locust bean gum, sacran, or a salt thereof. Suitable concentration of these polysaccharide concentration is 0.5 to 5 w/v % and the pH value of the formulation is between 3-8. In one example, 1-2% xanthan gum (KELTROL, CGT, CP Kelco company) is used alone as the pseudoplastic viscosity enhancing agent or in combination with 2% sodium alginate as in situ gelling matrix in the formulation.

Additional examples and procedures of making these in situ gelling matrix can be found in publications such as those described in DOI:10.15406/japlr.2016.02.00022, PMID:24120893, doi.org/10.1016/S0920-4105(00)00034-6, their related citations and the reference listed within the current invention and can be readily adopted for the current invention.

Exemplary formulations may comprise: a) cell surface anchoring antigen conjugate and immune function enhancing agents as described herein; b) pharmaceutically acceptable carrier; and c) polymer (e.g. a hydrophilic polymer) as matrix network, wherein said compositions are formulated as sustained release formulation such as implant or viscous liquids, i.e., viscosities from several hundred to several thousand cps or higher, gels or ointments; or formulated as an in situ gelling system as sustained release formulation. In these embodiments, the cell surface anchoring antigen conjugates is dispersed or dissolved in an appropriate pharmaceutically acceptable carrier.

Suitable dosages can be determined by standard methods, for example by establishing dose-response curves in laboratory animal models or in clinical trials and can vary significantly depending on the patient condition, the disease state being treated, the route of administration and tissue distribution, and the possibility of co-usage of other therapeutic treatments. The effective amount to be administered to a patient is based on body surface area, patient weight or mass, and physician assessment of patient condition. In various exemplary embodiments, a dose ranges from about 0.0001 mg to about 10 mg. In other illustrative aspects, effective doses ranges from about 0.01 μg to about 1000 mg per dose, 1 μg to about 100 mg per dose, or from about 100 μg to about 50 mg per dose, or from about 500 μg to about 10 mg per dose or from about 1 mg to 10 mg per dose, or from about 1 to about 100 mg per dose, or from about 1 mg to 5000 mg per dose, or from about 1 mg to 3000 mg per dose, or from about 100 mg to 3000 mg per dose, or from about 1000 mg to 3000 mg per dose. In other illustrative embodiments, effective doses can be about 1 μg, about 10 μg, about 25 μg, about 50 μg, about 75 μg, about 100 μg, about 125 μg, about 150 μg, about 200 μg, about 250 μg, about μg, about 300 μg, about 350 μg, about 400 μg, about 450 μg, about 500 μg, about 550 μg, about 575 μg, about 600 μg, about 625 μg, about 650 μg, about 675 μg, about 700 μg, about 800 μg, about 900 μg, 1.0 mg, about 1.5 mg, about 2.0 mg, about 10 mg, about 100 mg, or about 100 mg to about 30 grams. In certain embodiments, the dose is from about 0.01 mL to about 10 mL. In certain embodiments, the dose is administered to the subject in need thereof on daily basis as an injection. In other embodiments, the dose is given to the object once every 2-3 days as injection. In other illustrative embodiments, the dose is administered to the subject in need thereof once each week as an injection. In other embodiments, the dose is administered to the subject in need thereof once every two weeks as an injection. In other embodiments, the dose is administered to the subject in need thereof once every month as an injection. The treatment can be continued until the desired therapeutical effect is reached.

In some embodiments, the formulations contain 1˜100 mg/mL cancer cell inactivating agent/antigen presenting booster (e.g. Herceptin mimotope-cholesterylamine conjugate or native antigen-cholesterylamine conjugate such as those in U.S. patent application Ser. Nos. 15/945,741 and 16/271,877, or their mixture at 1:1 molar ratio, or the conjugate described in FIGS. 11-18), 0.1˜50 mg/mL TLR7/8 agonist (e.g. imiquimod or gardiquimod or resiquimod), 0.1˜50 mg/mL TLR3/RLR agonist (e.g. dsRNA such as poly IC or polyICLC), 0.1˜50 mg/mL TLR9 agonist (e.g. CpG ODNs such as ODN 1826 or ODN 2216) and optional 0.1˜50 mg/mL neuraminidase (sialidase) from Vibrio cholera and optional 0.1˜50 mg/mL Herceptin in water or 0.1-1×PBS, optional self-gelling material such as 5% sodium alginate, then being lyophilized to give the final formulation. In one example, the formulations contain 30 mg/mL said cancer cell inactivating agent/antigen presenting booster (e.g. the conjugate in FIGS. 11-18), 5 mg/mL imiquimod, 5 mg/mL poly IC, 5 mg/mL class A CpG ODN 2216, optional 100 mg/mL Herceptin, and optional 5 mg/mL neuraminidase from Vibrio cholera in 0.1×PBS and 5% sodium alginate. It can be injected to the tumor at 50˜300 μL/cm³ tumor size. In another example, the formulations contain optional 100 mg/mL said cancer cell inactivating agent/antigen presenting booster, 2 mg/mL imiquimod, 2 mg/mL poly IC, 2 mg/mL class A CpG ODN 2216 or class B CpG ODN, 20 mg/ml Herceptin (or antibody against other tumor surface antigen such as antibody against PD-L1 or antibody against EpCAM including IgG and IgM) and optional 2 mg/mL neuraminidase-lipid conjugate in 4% sodium alginate. The water insoluble drug such as imiquimod can be milled and dispersed in the formulation as suspension. Optional surfactant, e.g. 0.1˜1% tween-20 can be added to improve the solubility.

In some embodiments, the formulations contain 10˜100 mg/mL cancer cell inactivating agent/antigen presenting booster (e.g. Herceptin epitope/mimotope-cholesterylamine conjugate or native antigen-cholesterylamine conjugate such as those in U.S. patent application Ser. Nos. 15/945,741 and 16/271,877, or their mixture at 1:1 molar ratio, or the conjugate described in FIGS. 11-18), 1˜50 mg/mL STING agonist such as ADU-S100 or MK-1454 or SB 11285, 0.1˜50 mg/mL TLR7/8 agonist (e.g. imiquimod or gardiquimod or resiquimod), 1˜50 mg/mL TLR3/RLR agonist (e.g. dsRNA such as poly IC or polyICLC), 1˜50 mg/mL TLR9 agonist (e.g. CpG ODNs such as ODN 1826 or ODN 2216) and optional 1˜10 mg/mL neuraminidase from Vibrio cholera and optional 1˜50 mg/mL Herceptin in water or 0.1˜0.5×PBS, either as liquid formulation or being lyophilized to give the final formulation with optional 2% sucrose or mannitol. Said formulations can further comprise 3˜9% sodium alginate with optional 1-2% HPMC to be an ion triggered in situ gelling formulation. Instead of alginate, other in situ gelling material such as 15% Pluronic F127 with 0.1% chitosan, or 17˜20% poloxamer 407 can be comprised within said formulations to make a thermosensitive in situ gelling formulation. Another in situ gelling material can be used in the said formulation is using PLGA dissolved in biocompatible solvent such as N-methyl pyrrolidone or DMSO as matrix (e.g. those in PMID: 24929039), said formulations can be dissolved/dispersed in the PLGA solution (e.g. 10%˜50% PLGA in N-methyl pyrrolidone) or two components are combined immediately before injection. After injection, a solid implant will form. In one example, the formulations contain 30 mg/mL cancer cell inactivating agent/antigen presenting booster (e.g. Herceptin mimotope-cholesterylamine conjugate or Herceptin mimotope-cell membrane anchoring peptide conjugate or native antigen-cholesterylamine conjugate, or the conjugate described in in FIGS. 11-18), 1 mg/mL ADU-S100 or MK-1454, 2 mg/mL imiquimod, 2 mg/mL poly IC, 2 mg/mL class A CpG ODN 2216, 50 mg/mL Herceptin, and optional 1 mg/mL neuraminidase from Vibrio cholera in water and 5% sodium alginate. It can further containing 3% sucrose and then be lyophilized. It can be injected into the tumor at 100˜300 μL/cm³ tumor size or 0.1˜3 mL/tumor (after being reconstituted with water if it is lyophilized). In another example, the formulations contain 100 mg/mL cancer cell inactivating agent/antigen presenting booster, 2 mg/mL STING agonist MK-1454 or SB 11285, 2 mg/mL imiquimod or 1 mf/mL resiquimod, 2 mg/mL poly IC, 2 mg/mL class A CpG ODN 2216 or SD-101, 20 mg/ml Herceptin. 5% sodium alginate and optional 2 mg/mL neuraminidase-lipid conjugate in 0.1×PBS and 15% mineral oil to form an emulsion.

In one example, the formulations contain 30 mg/mL said cancer cell inactivating agent/antigen presenting booster, optional 1-2 mg/mL ADU-S100 or MK-1454, 2 mg/mL imiquimod, 5 mg/mL poly IC, 5 mg/mL classe A CpG ODN 2216, optional 50 mg/mL Herceptin, and optional 5 mg/mL neuraminidase, 3% sodium alginate, 0.5% calcium gluconate solution in water. It can be injected to the tumor at 0.1˜2 mL/tumor. In another example, the formulations contain 100 mg/mL said cancer cell inactivating agent/antigen presenting booster, 0.2 mg/mL STING agonist MK-1454 or SB 11285, 0.2 mg/mL imiquimod, 0.2 mg/mL poly IC, 0.2 mg/mL class A CpG ODN 2216 or class B CpG ODN, 10 mg/ml Herceptin or cetuximab and optional 2 mg/mL neuraminidase-lipid conjugate in 5% sodium alginate, 1% CMC, 0.2 Tween-60 and 5% mineral oil to form an emulsion.

The drugs in the said embodiments are in active form, one or more or all of them can also be in the form of prodrug, liposome, micelle, insoluble precipitate (e.g. in complex with condensing agent), conjugated to polymer drug carrier (e.g. dextran) or coated/adsorbed on or encapsulated in biodegradable micro particle/nano particle as previously described. For example, compounds having one or more amine groups that can precipitate poly IC or CpG ODN or CDN type STING agonist therefore generate water insoluble precipitates that can be used as sustained release drug form for the current invention. Examples of said co-precipitation compound can be found in U.S. patent application Ser. Nos. 15/945,741 and 16/271,877. Imiquimod or gardiquimod or resiquimod can also form precipitation with poly IC or CpG ODN or CDN type STING agonist or other anionic polymer or anionic lipid or anionic surfactant, which can be used in the current invention. Surfactant can be added to the precipitates to from stable suspension. Cationic lipid and other co-precipitation compound can also be used as co-precipitation anionic compound as disclosed in U.S. patent application Ser. Nos. 15/945,741 and 16/271,87. They can be mixed with negatively charged poly IC or CpG ODN or CDN type STING agonist or to form water insoluble complex (precipitation in water) to be used as intratumoral injection. The formed complex can also be encapsulated in biodegradable micro particle/nano particle and then being injected intratumorally to treat cancer. Another type of condensing agent is cationic solid such as nano or micro particles, which will bind with negatively charged anionic poly IC or CpG ODN or STING agonist to form water insoluble complex as sustained release system for the current invention, which are also described in U.S. patent application Ser. Nos. 15/945,741 and 16/271,877.

Encapsulation of poly IC or CpG ODN or STING agonist in biodegradable micro or nano sphere can be performed by the addition of amine containing compounds described as those in U.S. patent application Ser. Nos. 16/271,877 and 16/924,184. The prepared nanosphere can be used as vaccine adjuvant for the current invention. In another example, the nanosphere encapsulating poly IC and ADU-S100 and imiquimod is prepared using a double emulsion water/oil/water system as described in U.S. patent application Ser. No. 16/924,184.

In some embodiments, the drug containing PLGA particles are in micro meter size (e.g. 1-30 um in diameter) and the protocol of preparation can be found in reference and adopted for the current invention readily. Other examples of preparing TLR agonist containing particle or precipitations can be found in the disclosure of U.S. patent application Ser. No. 15/945,741. For example, PLGA TLR agonist microparticles are synthesized using PLGA polymer (PLGA, 50:50 or 65:35, molecular weights from 10,000-85,000 Da), using single emulsion method. Briefly, 100 mg PLGA is dissolved in 2 mL dichloromethane (DCM) with 10 mg imiquimod and homogenized in 1% poly vinyl alcohol (PVA, 10 mL, 8789% hydrolyzed, MW 13,000˜23,000 kDa, Sigma #363170) at 2000 rpm. This solution is added to 1% PVA 100 mL and is allowed to stir continuously for 3-4 h to evaporate DCM completely. The solution is then centrifuged at 11,000 g and washed using deionized water twice to wash away the excess PVA. The microcapsules are wet sieved to collect the desired diameter particles (e.g. 1-10 μm). The microparticles are then re-suspended in deionized water and rapidly frozen at −80° C. followed by lyophilization. The bigger the particle, the slower the drug release rate. In some embodiments, antibody against tumor cell surface antigen (e.g. Herceptin or anti PD-L1 antibody) is further coated to the nano or micro particles encapsulating TLR agonist (or STING agonist). The protocol to coat (e.g. chemical conjugation) antibody is well known to the skilled in the art and can be readily adopted for the current invention. Those antibody coated particles can be injected intratumorally to treat cancer or injected into or proximal to the tumor draining lymph node to treat cancer or applied to the tumor removed site during surgery to treat cancer or given systematically (e.g. IV injection) to treat cancer.

Tumor antigen binding antibody coated particle encapsulating PRR agonist (e.g. TLR agonist, STING agonist) is another type of cancer cell inactivating agent and is essentially the combination of cancer cell inactivating agent and immune function enhancing agent. It can be incorporated within the sustained release formulations of the current invention either alone to replace the combination of other cancer cell inactivating agent and immune function enhancing agent, or be together with other immune function enhancing agent in the formulation, to be injected intratumorally to treat cancer or injected into or proximal to the tumor draining lymph node to treat cancer or applied to the tumor removed site during surgery to treat cancer. In some embodiments, sustained release formulation is not required as the particle form can provide sustained release by itself.

Synthesis of antibody coated NP containing TLR agonist: in one example, PLA-PEG-MAL is synthesized according to publication of PMID: 24285486. To prepare the nano particles (NPs) encapsulating TLR agonist, 10 mg of PLA-PEG-MAL in 1 ml of acetonitrile containing 1 mg R848 is added dropwise to 5 ml of water. The solution is mixed for 2 hours, and the NPs is purified by filtration with Millipore Amicon Ultra 100,000 NMWL. The NPs are washed twice with water and twice with PBS containing 5 mM EDTA. Concurrently, 50 mg of anti HER2 antibody Trastuzumab in PBS containing 5 mM EDTA is reacted with 0.5 ml of 2-iminothiolane (5 mg/ml) for 1 hour. The modified antibody is added to the NPs and mixed for 1 hour to allow conjugation at 4° C. utilizing the maleimide-thiol reaction for conjugation. The NP-antibody is washed with PBS using Millipore Amicon Ultra 100,000 NMWL, generating the purified antibody coated NP with R848 encapsulated within. A formulation containing 5% NP in PBS or 5% in 3% sodium alginate is used to treat HER2 positive tumor.

In another example, 500 mg of PLGA (50:50 glycolide/lactide, MW=17000) with terminal COOH groups and 500 mg of PLGA-NHS (PLGA-N-hydroxysuccinimide, MW 50,000-80,000 Da, LA:GA 50:50) is dissolved in dichloromethane to generate a solution of 5 mg/mL total PLGA. R837 (TLR7 ligand) is dissolved in DMSO at 2.5 mg/ml. A total of 50 μL R837 is added to 1 ml said PLGA (5 mg/ml) dissolved in dichloromethane. Next the mixture is homogenized with 0.4 ml 5% w/v PVA solution for 10 min using ultrasonication. The o/w emulsion is added to 2.1 ml of a 5% w/v solution of PVA to evaporate the organic solvent for 4 h at room temperature. PLGA-R837 nanoparticles are obtained after centrifugation at 3,500 g for 20 min. The PLGA-R837 nanoparticles have surface —COOH and NHS group, which can react with the amine group of antibody for conjugation. 1 ml of 5% PLGA-R837 nanoparticles is mixed with 1 mL 5% anti-PD-L1 humanized IgG1 antibody (e.g. avelumab, durvalumab, atezolizumab) and 20 mg EDAC, 5 mg NHS in pH 8.5 50 mM NaHCO₃—Na₂CO₃ buffer for 2 h for antibody conjugation to the NP. The resulting antibody coated NP is purified with centrifugation or filtration. A formulation containing 5% purified antibody coated PLGA-R837 NP in PBS or in 3% sodium alginate can be used to treat PD-L1 positive tumor.

Alternatively, the TLR agonist or other immune enhancing agent can be conjugated to the particles instead of being encapsulated. The conjugation can be either on the surface of the particle or use the polymer-immune enhancing agent conjugate to form the particle. For example, TLR agonist-PLA conjugate can be used to synthesize the NP. In one example, PLA-R848 conjugate is prepared according to US patent application US20170349433. Next mg of PLGA (50:50 glycolide/lactide, MW=17000) with terminal COOH groups and 200 mg of PLGA-NHS (MW 50,000-80,000 Da, LA:GA 50:50) and 600 mg conjugate is dissolved in dichloromethane to generate a solution of 5 mg/mL total PLGA. Next the mixture is homogenized with 0.4 ml 5% w/v PVA solution for 10 min using ultrasonication. The o/w emulsion is added to 2.1 ml of a 5% w/v solution of PVA to evaporate the organic solvent for 4 h at room temperature. PLGA-R848 nanoparticles are obtained after centrifugation at 3,500 g for 20 min. The PLGA-R848 nanoparticles have surface —COOH and NHS group, which can react with the amine group of antibody for conjugation.

Similarly, tumor antigen binding antibody-PRR agonist conjugate, an antibody-drug conjugate, is another type of cancer cell inactivating agent and is essentially the combination of cancer cell inactivating agent and immune function enhancing agent. It can be incorporated within the sustained release formulation of the current invention either alone or together with other immune function enhancing agent to be injected intratumorally to treat cancer or injected into or proximal to the tumor draining lymph node to treat cancer or applied to the tumor removed site during surgery to treat cancer.

Examples of the tumor antigen binding antibody (antibody against tumor cell surface antigen) and PRR agonist are disclosed throughout in the current invention.

Besides TLR agonist and STING agonist, other molecules that can activate/boost the function of immune system and immune cell such as APC, B cell and T cells can also be incorporated into the formulations in the current invention or used alone to be injected into tumor or injected into or proximal to the tumor draining lymph node or applied to the tumor removed site during surgery to treat cancer. Suitable immune function activating/boosting molecule can be selected from those disclosed in U.S. patent application Ser. Nos. 15/945,741, 16/271,877 and 16/924,184, e.g. checkpoint inhibitor such as antibody against them, granulocyte macrophage colony-stimulating factor, immunostimulatory antibody (e.g. anti-KIR antibody such as lirilumab, antibody for CD137 such as urelumab or utomilumab), heparan sulfate (HS) mimetics such as PG545 (pixatimod), FMS-like tyrosine kinase 3 ligand (FLT3L), other pattern recognition receptor agonists besides poly IC, CpG and imiquimod, T-cell-tropic chemokines such as CCL2, CCL1, CCL22 and CCL17, B-cell chemoattractant such as CXCL13, interferon gamma, type I IFN (e.g. IFN-α, IFN-beta), TNF-beta, TNF-alpha, immunity boosting interleukins such as IL-1, IL-2, IL-12, IL-6, IL-12, IL-24, IL-18, IL-5, IL-6, IL-9, IL-21 and IL-15 or their derivatives such as PEGylated derivative, CD1d ligand, Vα14/Vβ8.2 T cell receptor ligand, iNKT agonist, HS44, α-GalCer, α-GlcCer, α-glucuronylceramide, α-galacturonylceramide, isoglobotriosylceramide, CD40 agonist such as APC activating anti-CD40 antibody, CD154 or its mimic, antibody against OX40, Pcsk9 antibody such as evolocumab or alirocumab, antibody against TGF-β including bispecific antibody against TGF-β and CD4 such as CD4TGF-βTrap (4T-Trap), CD96 agonist such as CD96 activating antibody, CD28 agonist such as CD28 activating antibody, antibody against VISTA, antibody against CD39 or against CD73 or against A2aR, immunomodulatory imide drugs (immune enhancing IMiDs such as thalidomide, lenalidomide and pomalidomide), antibody blocking SIRPα, antibody against CD47, Treg inhibitory agent such as inhibitory antibody against Treg (such as antibody against CD4, CD25, FOXP3 and TGF-β or its receptor) or their combinations. Another type of immune activating/boosting molecules that can be used include super antigen (e.g. staphylococcal enterotoxin A, SEA and those described in patent application U.S. Ser. No. 15/373,483 and CN102391377A) as well as interleukin superagonist such as interleukin-15 superagonist (e.g. N-803) and other immunity activating interleukin superagonist formed by interleukin with its receptor or interleukin with antibody, such as IL-2/anti-IL-2 antibody complex, IL-6/anti-IL-6 antibody complex, IL-3/anti-IL-3 antibody complex, IL-7/anti-IL-7 antibody complex, IL-15/anti-IL-15 antibody complex; IL-2/anti-IL-2 receptor complex, IL-6/anti-IL-6 receptor complex, IL-3/anti-IL-3 receptor complex, IL-7/anti-IL-7 receptor complex. Those complex can be a single fused protein. They can be added to the formulation described in the current invention at therapeutically effective amount, to be used as an intratumoral injection or as implant or being injected into or proximal to the tumor draining lymph node or applied to the tumor removed area.

In one example, the formulation is a solution containing 20˜200 mg/mL cetuximab mimotope-cholesterylamine conjugate or cetuximab mimotope-cell membrane anchoring peptide conjugate, 0.2-2 mg/mL MK-1454 or 0.3-3 mg/mL poly IC or 0.3-3 mg CpG ODN 2216 or their combination, 20 mg/mL biodegradable PLGA micro particles encapsulating 5-20% imiquimod, optional 5-100 mg/mL cetuximab and granulocyte-monocyte colony-stimulating factor (10-200 μg/mL) and optional sodium alginate 3%˜9%. Suitable amount of surfactant can be added to from stable suspension. In another example, the formulation is a solution containing 20˜200 mg/mL cetuximab mimotope-cholesterylamine conjugate or cetuximab mimotope-cell membrane anchoring peptide conjugate, 0.2 mg/mL MK-1454 or 0.3 mg/mL poly IC or 0.3 mg CpG ODN 2216 or their combination, 20 mg/mL biodegradable PLGA nano particles encapsulating 10% imiquimod, optional 5-50 mg/mL cetuximab and granulocyte-monocyte colony-stimulating factor (10-200 μg/mL) with optional 5% sodium alginate. After the patient receive the intratumoral injection and/or injection into or proximal to the tumor draining lymph node with either above formulation at 0.1-0.5 mL/cm³ tumor volume, the patient is intravenously injected with cetuximab immediately 3˜10 mg/kg once and ipilimumab 3˜10 mg/kg every 3 weeks for 4 doses, or atezolizumab 1200 mg IV q3wk to treat cancer. Cetuximab 3˜10 mg/kg can also be is intravenously injected before the intratumoral injection of the above formulation. 10˜50 mg/mL L-rhamnose-cholesterylamine conjugate can also be added to the formulation.

1260 In another example, the composition is a solution containing 50-100 mg/mL Herceptin mimotope-lipid conjugate with optionally 50 mg/mL Herceptin, 5 mg/ml ADU-S100, 10 mg/mL imiquimod, 2 mg/mL poly IC, 2 mg/mL CpG ODN 2216, 1×10⁴-1×10⁵ U/mL of IFN-α, 1-10 MIU/mL IL-2, L-arginine, L-cysteine and L-tryptophan at 20˜100 mg/mL, poly aspirin 20 mg/mL, glutathione or SOD 5 mg/mL, N-hydroxy-L-arginine 10 mg/mL, tadalafil 3 mg/mL, axitinib 10 mg/mL, nitro-aspirin 5 mg/mL, all-trans retinoic acid 5 mg/mL, 5 mg/mL α-GalCer, gemcitabine 10 mg/mL, cucurbitacin 10 mg/mL and 6% sodium alginate. Suitable amount of hyaluronic acid is added to the solution to reach a viscosity of 100,000-1,000,000 cps. After the patient receive the intratumoral injection with the above formulation, the patient is intravenously injected with ipilimumab 3˜10 mg/kg every 3 weeks for 4 doses, or atezolizumab 1200 mg IV q3wk until disease progression. Herceptin 5-10 mg/kg can also be intravenously injected before or after the intratumoral injection of the above formulation. In another example, the composition that can be used the same way is a solution containing 50-mg/mL Herceptin mimotope-lipid conjugate with optionally 100 mg/mL Herceptin, 0.5-5 mg/ml ADU-S100, 1 mg/mL imiquimod, 0.2-2 mg/mL poly IC, 0.2-2 mg/mL CpG ODN 2216, 1×10⁴-1×10⁵ U/mL of IFN-α, 1-10 MIU/mL IL-2, L-arginine, L-cysteine and L-tryptophan at 20˜100 mg/mL, poly aspirin 20 mg/mL, glutathione or SOD 5 mg/mL, N-hydroxy-L-arginine 10 mg/mL, tadalafil 3 mg/mL, axitinib 10 mg/mL, nitro-aspirin 5 mg/mL, all-trans retinoic acid 5 mg/mL, 5 mg/mL α-GalCer, gemcitabine 10 mg/mL, cucurbitacin 10 mg/mL in 5% sodium alginate or 10-15% poloxamer 407. Suitable amount of carbomer such as Carbopol 934 is added to the solution to reach a viscosity of 100,000-1,000,000 cps. After the patient receive the intratumoral injection with the above formulation, the patient is intravenously injected with ipilimumab 3˜10 mg/kg every 3 weeks for 4 doses, or atezolizumab 1200 mg IV q3wk until disease progression. Herceptin 5-10 mg/kg can also be intravenously injected before or after the intratumoral injection of the above formulation.

1285 In another example, the formulation is a solution containing 100˜200 mg/mL PLGA nano particles encapsulating 20% Herceptin mimotope-lipid conjugate, 2 mg/mL antibody against OX40, 2 mg/mL antibody against PD-L1, 2 mg/mL poly IC, 2 mg/mL CpG ODN 2216, 1 mg/ml ADU-S100, 5 mg/mL imiquimod, 0.5-2 mg/mL α-GalCer, 25×10⁴ U/mL of IFN-gamma, 25×10⁴ U/mL of IFN-α, 5 MIU/mL IL-2 in 5% sodium alginate or 15% poloxamer 407. After the patient receive the intratumoral injection with the above formulation at 0.5-2 mL/tumor, the patient is intravenously injected with ipilimumab 3˜10 mg/kg every 3 weeks for 4 doses, or atezolizumab 1200 mg IV q3wk until disease progression. Herceptin 5-10 mg/kg is intravenously injected right before or right after the intratumoral injection of the above formulation. Alternatively, the formulation as injection is a solution containing 50˜100 1295 mg/mL PLGA nano particles encapsulating 20% Herceptin mimotope-folate conjugate and 10% imiquimod or 3% R848, 20˜100 mg/mL alpha-gal-cholesterylamine conjugate, 2 mg/mL poly IC, 2 mg/mL SB 11285, 2 mg/mL CpG ODN 2216, 1 mg/mL 3M-052 in 5% sodium alginate or 17% poloxamer 407.

In another example, the formulation is a solution containing 50-100 mg/mL Fc-lipid conjugate or FcMBL or the conjugate described in FIGS. 11-18, 10 mg/mL imiquimod, 2 mg/mL poly IC, 1 mg/mL SB11285, 2 mg/mL CpG ODN 2216, 50 μg/mL granulocyte-monocyte colony-stimulating factor, 1×10⁴-1×10⁵ U/mL of IFN-gamma, 1-10 MIU/mL IL-2 in 6% sodium alginate and 0.5% CMC. After the patient receive the intratumoral injection with the above formulation at 0.5-3 mL/tumor, the patient is intravenously injected with ipilimumab 3˜10 1305 mg/kg every 3 weeks for 4 doses, or atezolizumab 1200 mg IV q3wk until disease progression.

The liquid and solution in the current invention is aqueous liquid if not specified. The drug (e.g. TLR agonist, cancer cell inactivating agent) in the liquid formulation can be either in the form of solubilized drug or insoluble form such as aggregate, particles including crystals, precipitations. In some embodiments, the drug in the liquid form is present as suspension. Some drug such imiquimod, 3M-052 has low water solubility, they can be present in the liquid form as fine particle suspensions. Additional aqueous solubility-enhancing excipient can be added to the formulation to improve the solubility of poorly water soluble drug, such as suitable amount of surfactant (e.g. 0.05%˜0.5% tween-20, tween-60, tween-80, lecithin, spans, fatty acid esters of glycerol, alkyl polyglucosides), polymers (e.g. 0.2-2% PVA, 1%-10% PEG), organic solvent as co-solvent (e.g. 2-20% ethanol, DMSO, propylene glycol).

In another example, the formulation is a solution containing 100 mg/mL Herceptin mimotope NHS ester, 3 mg/mL ADU-S100, 10 mg/mL imiquimod, 2 mg/mL poly IC, 2 mg/mL CpG ODN 2216 in 30% PLGA N-methyl pyrrolidone solution. After the patient receive the intratumoral injection with the above formulation, the patient is intravenously injected with ipilimumab 3˜10 mg/kg every 3 weeks for 4 doses, or atezolizumab 1200 mg IV q3wk until disease progression. Herceptin 5-10 mg/kg can also be intravenously injected before or after the intratumoral injection of the above formulation.

Instead of the antigen containing conjugate described above, another type of cancer cell inactivating agent can be used in the composition/formulation/method to treat cancer described in the embodiments and examples of U.S. patent application Ser. Nos. 15/945,741, 16/271,877 and 16/924,184 and current invention is therapeutic antibody including monoclonal antibody, bispecific antibody, tri-specific antibody and antibody-drug conjugate as well as cytotoxic T cell used to treat cancer. Examples of therapeutic antibody (including antibody-drug conjugate) include Herceptin, rituximab, Bexxar, cetuximab, bevacizumab, panitumumab, pertuzumab, Kadcyla and catumaxomab, antibody against VEGF or VEGFR or EGFR. It can also be antibody against tumor surface antigen such as GalNAc-O-Ser/Thr (Tn Antigen), Gal 1-3GalNAc-O-Ser/Thr (core 1 antigen), STF Antigen, PSA, PSCA and etc.

For example, antibody against PSA (prostate-specific antigen) can be used for prostate cancer. Antibody against PSCA can be used to treat solid tumors expressing prostate stem cell antigen (PSCA). Other examples of the antibody can be used include those clinically used or under development antibody drug against tumor cell surface antigen such as those described previously for the formulation applied to tumor removal site and embodiments type 1. Additional target of the antibody can be found in US patent application U.S. Ser. No. 10/675,358. Preferably the formulation is a sustained release formulation such as an in-situ gelling formulation or implant such as those disrobed in the current invention. The antigen need not to be highly tumor specific because the antibody can be injected into the tumor to reach high local concentration to be effective. For example, antibody against epithelial cell adhesion molecule (EpCAM) antigen can be used for epithelia and epithelial-derived tumor cells although it also binds with other normal epithelial cells. Additional highly abundant cell surface protein and cell surface carbohydrate that can be used as tumor antigen are disclosed in U.S. patent application Ser. Nos. 16/271,877 and 16/924,184.

Either single antibody or combination of several antibodies against different tumor surface antigen can be used in the compositions and formulations of the current inventions to treat cancer. For example, an in situ gelling formulation containing TLR agonist and a mixture of antibody against HER2 and antibody against PD-L1 and be used to treat tumor containing HER2+ cells and PD-L1+ cells. In another example, an sustained release formulation containing TLR agonist and a mixture of antibody against EpCAM, antibody against PD-L1 and antibody against sialic acid is used to treat tumor containing EpCAM+ cells, PD-L1+ cells and sialic acid+ cells. In one example, the in-situ gelling composition/formulation is a solution containing about 10 mg/mL IgG1 or IgM antibody against EpCAM, 10 mg/mL anti PD-L1 antibody avelumab, 10 mg/mL trastuzumab against HER2, 5 mg/mL poly IC, optional 1 mg/mL imiquimod, 3.5% sodium alginate with pH between 6-8 and osmolarity adjusted to 250˜350 mOsm/kg with NaCl, to treat cancer expressing those antigens.

The antibody can be antibody fragment, IgG, IgA, IgE, IgM, IgG-IgA chimeric, polymeric antibody (e.g. multiple antibody or its fragment conjugated to a polymer backbone), antibody coated nano or micro particle, antibody-drug conjugate, bispecific antibody, tri-specific antibody, tetra-specific antibody or higher degree multi-specific antibody as well as their derivatives. Preferably the antibody has low tendency to be internalized by the cancer cell. In some embodiments endocytosis inhibitors, such as prochlorperazine, chloroquine and hydroxychloroquine can be incorporated in the formulation at 0.05%˜5% to inhibit the antibody internalization.

In some embodiments, when the antibody is applied non-systemically (injected intratumorally or injected into or proximal to the tumor draining lymph node or applied to the tumor removed site), preferably the antibody has a shorter systemic half-life than that of natural IgG1. It can be a IgG3 or its derivatives. It can have a modified Fc region to reduce its in vivo half-life, which can be done by reducing its binding to FcRn receptor at slightly acidic pH (5-6.5) or enhance its binding to FcRn receptor at neutral pH or both with Fc engineering. There are many strategy such as point mutations that can reduce antibody's in vivo half-life, which can be found in many publications and readily adopted in the current invention. This will reduce the side effect of antibody when it is leaked into systemic circulation to provide better safety profile. Preferably the antibody has high antibody-dependent cellular phagocytosis (ADCP) activity. The antibody can be engineered to improve their ADCP activity, such as modification on their Fc portion. Examples of these engineering strategies are disclosed below.

In some embodiments, the antibodies contain a modified Fc region, wherein the modification modulates the binding of the Fc region to one or more Fc receptors, to enhance its ADCC/ADCP activity. The terms “Fc receptor” or “FcR” refer to a receptor that binds to the Fc region of an antibody. There are three main classes of Fc receptors: FcγR which bind to IgG, FcαR which binds to IgA, and FcεR which binds to IgE. The FcγR family includes several members, such as FcγI (CD64), FcγRIIA (CD32A), FcγRIIB (CD32B), FcγRIIIA (CD16A), FcγRIIIB (CD16B). FcγRIIB is a inhibitory receptor and others are activating receptors but agonistic anti-TNFR antibodies often require FcγRIIB engagement.

In some embodiments, the antibodies contain one or more modifications (e.g., amino acid insertion, deletion, and/or substitution) in the Fc region that results in modulated binding (e.g., increased binding or decreased binding) to one or more Fc receptors (e.g., FcγRI (CD64), FcγRIIA (CD32A), FcγRIIB (CD32B), FcγRIIIA (CD16a), and/or FcγRIIIB (CD16b)) as compared to the native antibody lacking the mutation in the Fc region. In some embodiments, the antibodies contain one or more modifications in the Fc region that reduce the binding of the Fc region of the antibody to FcγRIIB In some embodiments, the antibodies contain one or more modifications in the Fc region of the antibody that reduce the binding of the antibody to FcγRIIB while maintaining the same binding or having increased binding to FcγRI (CD64), FcγRIIA (CD32A), and/or FcRγIIIA (CD16a) as compared to the native antibody lacking the mutation in the Fc region. In some embodiments, the antibodies contain one of more modifications in the Fc region that increase the binding of the Fc region of the antibody to FcR activating receptors. Those modification can enhance the ADCC/ADCP activity of the antibody. In some cases, the modulated binding is provided by mutations in the Fc region of the antibody relative to the native Fc region of the antibody. The mutations can be in a CH2 domain, a CH3 domain, or a combination thereof. Native sequence human Fc regions include a native sequence human IgA Fc region, a native sequence human IgM Fc region, a native sequence human IgG1 Fc region; native sequence human IgG2 Fc region; native sequence human IgG3 Fc region; and native sequence human IgG4 Fc region as well as naturally occurring variants thereof. Native sequence Fc includes the various allotypes of Fcs. Those modification will enhance the ADCC and/or ADCP activity of the antibody.

In some embodiments, the mutations in the Fc region that result in modulated binding to one or more Fc receptors can include one or more of the following mutations: SD (S239D), SDIE (S239D/I332E), SE (S267E), SELF (S267E/L328F), SDIE (S239D/I332E), SDIEAL (S239D/1332E/A330L), GA (G236A), ALIE (A330L/1332E), GASDALIE (G236A/S239D/A330L/I332E), V9 (G237D/P238D/P271G/A330R), and V11 (G237D/P238D/H268D/P271G/A330R) and/or one or more mutations at the following amino acids: E233, G237, P238, H268, P271, L328 and A330. Examples of other Fc region modifications include AAA (S298A/E333A/K334A), afucosylation, DE(S239D/I332E), DLE (S239D/A330L/1332E), G236A, ADE (G236A/S239D/I332E), GAALIE(G236A/A330L/I332E), GASDALIE(G236A/5239D/A330L/I332E), LPLIL(F243L/R292P/Y300L/V305I/P396L), VLPLL(L235V/F243L/R292P/Y300L/P396L) and those described in publication of PMID: 33212886. Additional Fc region modifications for modulating Fc receptor binding are described, e.g., in U.S. Pat. Nos. 9,605,080, 7,416,726 and 5,624,821. These modification can enhance the ADCC/ADCP activity of the antibody.

In some embodiments, the Fc region of the antibodies are modified to have an altered glycosylation pattern of the Fc region compared to the native non-modified Fc region. Human immunoglobulin is glycosylated at the Asn297 residue in the Cy2 domain of each heavy chain. This N-linked oligosaccharide is composed of a core heptasaccharide, N-acetylglucosamine4Mannose3 (GlcNAc4Man3). Removal of the heptasaccharide with endoglycosidase or PNGase F is known to lead to conformational changes in the antibody Fc region, which can significantly reduce antibody-binding affinity to activating FcγR and lead to decreased effector function. The core heptasaccharide is often decorated with galactose, bisecting GlcNAc, fucose or sialic acid, which differentially impacts Fc binding to activating and inhibitory FcγR. Additionally, it has been demonstrated that a2,6-sialyation enhances anti-inflammatory activity in vivo while defucosylation leads to improved FcγRIIIa binding and a 10-fold increase in antibody-dependent cellular cytotoxicity and antibody-dependent phagocytosis. Specific glycosylation patterns can therefore be used to control inflammatory effector functions. Those modification can enhance the ADCC/ADCP activity of the antibody. In some embodiments, the modification to alter the glycosylation pattern is a mutation. For example, a substitution at Asn297. In some embodiments, Asn297 is mutated to glutamine (N297Q). Methods for controlling immune response with antibodies that modulate FcγR-regulated signaling are described, for example, in U.S. Pat. No. 7,416,726 as well as U.S. patent application Ser. Nos. 11/317,892 and 12/092,235. Those modification will enhance the ADCC/ADCP activity of the antibody.

In some embodiments, the antibodies are modified to contain an engineered Fab region with a non-naturally occurring glycosylation pattern. For example, hybridomas can be genetically engineered to secrete afucosylated mAb, desialylated mAb or deglycosylated Fc with specific mutations that enable increased FcRγIIIa binding and effector function. In some embodiments, the antibodies are engineered to be afucosylated (e.g., afucosylated rituximab, available from Invivogen, hcd20-mab13). Those modification will enhance the ADCC/ADCP activity of the antibody.

In some embodiments, the entire Fc region of an antibody is exchanged with a different Fc region, so that the Fab region of the antibody is conjugated to a non-native Fc region. For example, the Fab region of rituximab, which normally comprises an IgG1 Fc region, can be conjugated to IgG2, IgG3, IgG4, or IgA, or the Fab region of nivolumab, which normally comprises an IgG4 Fc region, can be conjugated to IgG1, IgG2, IgG3, IgA1 or IgG2. In some embodiments, the Fc modified antibody with a non-native Fc domain also comprises one or more amino acid modification, such as the S228P mutation within the IgG4 Fc, that modulate the stability of the Fc domain described. In some embodiments, the Fc modified antibody with a non-native Fc domain also comprises one or more amino acid modifications described herein that modulate Fc binding to FcR.

In some embodiments, the modifications that modulate the binding of the Fc region to FcR do not alter the binding of the Fab region of the antibody to its antigen when compared to the native non-modified antibody. In other embodiments, the modifications that modulate the binding of the Fc region to FcR also increase the binding of the Fab region of the antibody to its antigen when compared to the native non-modified antibody.

Additional examples of point mutations, glycoengineering, exchange of Fc domains across isotypes (cross-isotype antibodies) to enhance FcγR binding can be found in the publications of PMID:31231397 and PMID:28986820. These strategies can be readily applied to the antibody used in the current inventions. For example, single domains of IgA2 can be appended to end of the gamma 1 constant region creating a four-domain constant region (CH1g-CH2g-CH3g-CH3a) to engage FcγRs and FcαRI. To make the constant region more similar to the alpha constant region the CH1 domain of gamma 1 can be substituted for the alpha 1 constant region domain (CH1a-CH2g-CH3g-CH3a). In a similar approach a second type of cross-isotype Fc can be created by fusing the gamma 1 and alpha constant regions together to create a tandem G1-A Fc region, in which the hinge, CH2, and CH3 of IgA2 is fused to the C-terminus of IgG1. Alternatively, cross-isotype antibody by exchanging the CH3 domain and CH2 α1 loop residues 245-258 of the gamma 1 constant region with that of the alpha constant region can be created. IgG multimerization can also be used to augments FcγR binding. The IgG multimers can be constructed in various ways including IgG in tandem form, the addition of heterologous multimerization domains such as isoleucine zippers, another hinge region at the N-terminus of the natural hinge, or another hinge region at the C-terminus of the CH3 domain. Similarly, hexamers of IgG can be created by appending the IgM tailpiece to the C-terminus of the IgG1 Fc and creating a cysteine bond at position 309. Examples and protocols of them can be found in related publication PMID: 31231397 and similar antibody against tumor antigen can be prepared accordingly. Hexamer formation-enhanced (HexaBody) can also be used such as those described in PMID: 27474078 and similar antibodies against tumor surface antigens that can more efficiently form hexamers upon antigen binding can be constructed accordingly. The FIG. 1 in publication of PMID: 25970007 shows design of the IgG1/IgA2 tandem Fc fusion, a tandem IgG1/IgA2-Fc format with an IgA2-Fc and hinge fused to an IgG-Fc to generate enhanced ADCC and ADCP capabilities. Said antibody having IgG1/IgA2 tandem Fc fusion can be used in the current invention. This type of antibody that can bind with other tumor surface antigen target (e.g. EpCAM) can be readily prepared accordingly and used in the current invention.

In some embodiments, the antibody has a tandem format of Fc, e.g. one antibody molecule has multiple copy of Fc. Examples of the antibody having Fc multiplications can be found in publications of PMID:28102754, PMID: 18353438 and PMID: 21215693; similar antibody that can bind with other tumor surface antigen target (e.g. EpCAM) can be readily prepared accordingly and used in the current invention. More than 3 Fc can also be incorporated in the antibody. Each Fc region can be further engineered as described above to improve their FcγR binding and/or ADCP activity. Those modification will enhance the ADCC/ADCP activity of the antibody. FIG. 1 shows examples of antibody having tandem format of Fc that can be used in the current invention. The linker1+hinge and linker2+hinge can be the same as those described in publication of PMID:28102754. Alternatively, as shown in publications of PMID: 18353438 and PMID: 21215693, multiple Fc can be connected with linker having repeating GS rich sequence.

In some embodiments, another format of an antibody having two copy of native or engineered Fc is shown in FIG. 2. Examples of this kind of antibody can be found in publication of PMID: 31702857. FIG. 2 shows the design of 1Fc and 2Fc mAbs, whereas 1Fc mAb (A) contains the native configuration of two Fabs and one Fc region, 2Fc mAb (B) contains two each of Fab and Fc regions, (C) proteins are produced in HEK293 cells using expression plasmids containing the sequences for the mAb heavy and light chains (1Fc) or heavy chain and light chain-Fc fusion (2Fc). Duplication of the IgG Fc region allows for increased avidity to Fc receptors to improve their FcγR binding and ADCC/ADCP activity. Different antibody that can bind with different tumor surface antigen target (e.g. EpCAM) can be readily prepared accordingly and used in the current invention.

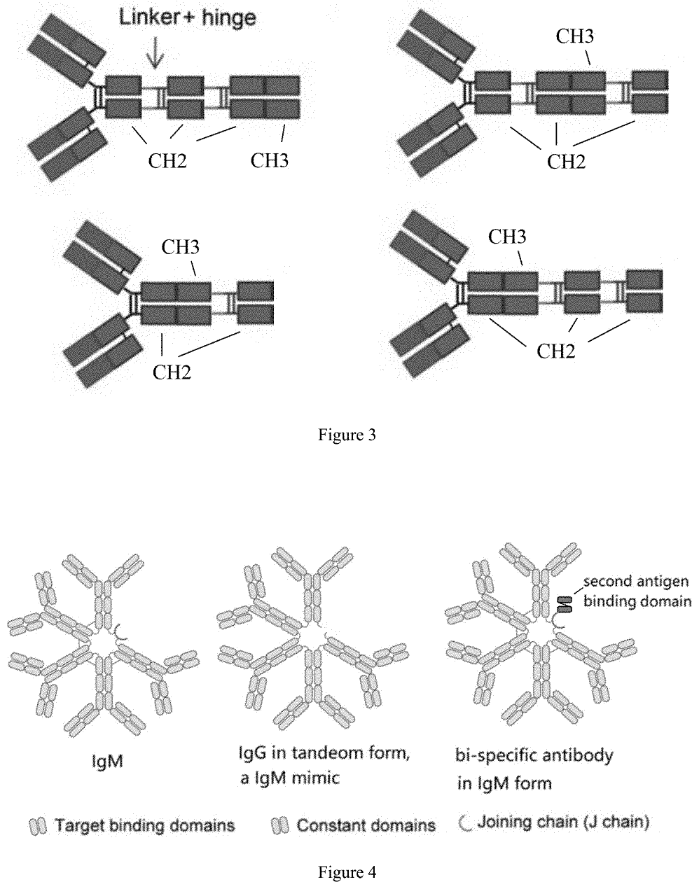

Because the FcγR binding is localized in Fc CH2 domain, another format of the antibody to improve their FcγR binding and ADCC/ADCP activity is to have repeated native or mutated CH2 region in the antibody instead of repeated full CH2-CH3 region of Fc as described above. Examples of antibody having 2 or 3 CH2 repeats are shown in the FIG. 3, antibody having more CH2 repeats can be constructed similarly. Different antibody that can bind with different tumor surface antigen target (e.g. EpCAM) can be readily prepared accordingly and used in the current invention.

The linker connecting the repeated CH2 or CH2CH3 in the antibody can be a longer than those shown in cited publications to allow better multivalent binding of Fc region to FcR. For example, it can be a flexible hydrophilic peptide having 20-100 AA (amino acids), such as (GₐS_{b})ₙ where in a, d and n are integers between 1-10. In some embodiments, the flexible peptide linker is a G/A/D/E rich peptide. Additional flexible liker can be found in U.S. patent application Ser. No. 16/364,113 by the current inventor. The hydrophilic peptide linker can be Asp, Glu, Ser/Gly/Ala rich peptide having 10˜200 AA, In some embodiments the peptide linker suitable for the current invention contains 10˜150 AA; preferably between 15˜100 AA. The sum of glycine (G), alanine (A), serine (S), threonine (T), glutamate (E), aspartate (D), and proline (P) residues can constitute more than about 90% of the total amino acid residues of linker; the sum of glutamate (E) and aspartate (D) residues constitutes more than about 20% of the total amino acid residues of linker. In some embodiments preferably the sum of glutamate (E) and aspartate (D) residues constitutes more than about 30% of the total amino acid residues of linker. Preferably the linker is flexible and displays a random secondary/tertiary structure. Example of this kind of linker can be found in U.S. patent application Ser. No. 16/364,113.

The antibody used in the current invention can also be IgE, IgE derivative, IgE mimics, IgM, IgM derivative and IgM mimics. They can be pentamer or hexamer, with or without J chain. They can be readily constructed based on the protocol in related publications such as those published by IgM Biosciences. FIG. 4 shows exemplary structures of an IgM pentamer targeting tumor antigen (e.g. EpCAM), a IgM mimic which is essentially a IgG in tandem form and a bispecific IgM that has a single-chain variable fragment (scFv) that can bind with antigen presenting cell surface marker such as FcγR.

In the current invention, multi-specific antibody includes bispecific antibody, tri-specific antibody, tetra-specific antibody or even higher degree multi-specific antibody. Tumor cell surface antigen targeting bispecific antibody, tri-specific antibody, tetra-specific antibody or higher degree multi-specific antibody as well as their derivatives and their conjugate with drug (e.g. TLR agonist, STING agonist) can also be used in the current invention. They can be used in the same way as the mono specific antibody in the embodiments and examples in the current invention. They can be used in a therapeutically effective amount to replace the cell surface anchoring antigen conjugate or mono specific antibody against tumor surface antigen in the sustained release formulations in the current invention such as an in-situ gelling formulation. In some embodiments, one or more arm of the multi-specific antibody can bind with tumor cell surface antigen, while the other arm/arms can bind with surface antigen of antigen presenting cells (e.g. their cell surface marker). In some embodiments, one or more arm of the multi-specific antibody can bind with tumor cell surface antigen, while the other arm/arms can bind with CD40 and can activate antigen presenting cells. Examples of antigen presenting cells include dendritic cells, macrophages, B cells, T cells and NK cells. For example, surface antigen of antigen presenting cells can be DC cell surface marker, e.g. CD11C; or CD14 on macrophage. Other examples of surface antigen of antigen presenting cell are pattern recognition receptors (PRRs) such as the Toll-like receptor, which are widely expressed on APC surface. Fc receptor is also an surface antigen of antigen presenting cell suitable for the current inventions, which include Fc-gamma receptors, Fc-alpha receptors and Fc-epsilon receptors. Examples of Fc-gamma receptors suitable for the multi-specific antibody include APC activating FcγRI (CD64), FcγRIIA (CD32), FcγRIIIA (CD16a) and FcγRIIIB (CD16b). Additional antigen/surface marker of DC that can be targeted by said multi-specific antibody can be found in the publication of PMID: 19197943, such as class II MHC, FIRE (F4/80-like receptor), CIRE, dectin-1, DCIR2, DEC-205 (CD205), mannose receptor, LOX-1, CD36, CD11c, C-type lectin receptors (e.g. dendritic cell immunoreceptor, DCIR), Clec9A, CD40, CD45, CD45RA, CD4 and DC-SIGN. Targeting CD40 can also activate DC cell when the antibody against CD40 is a CD40 agonist, e.g. an antibody that cause CD40 clustering.

These multi-specific antibody can also be engineered to further improve their ADCC/ADCP activity such as the engineering on their Fc portion if they have a Fc portion, using similar strategy described previously for mono specific antibody, e.g. point mutation, glycol engineering, tandem or hybrid Fc, CH2 repeating and multimerization.

An example of bispecific antibody is BsAb DF3xH22 disclosed in publication of PMID: 11358826, which can bind with human epithelial mucin, MUC-1 and Fc component of phagocytic cell. It can be used together with TLR agonist in an in-situ gelling system to treat MUC-1 positive tumor. Another example is BsAb MDX-H210 disclosed in publication of PMID: 9307286, which target sFcγ receptor type I (FcγRI, CD64) and HER2. Yet another example is BsAb HER2bsFab disclosed in publication of PMID: 24979648, which target FcγRIII and HER2. It can be used together with TLR agonist or STING agonist in sustained release formulation such as an in-situ gelling formulation to treat HER2 positive tumor.

There are many formats currently available for bispecific antibody and multi-specific antibody and these formats can be readily adopted to construct the antibodies to be used in the current inventions as long as they can provide the desired target binding as disclosed in the current inventions, for example, those bispecific antibody formats described in publications of PMID: 28071970 and PMID: 31175342. Exemplary formats of bispecific antibody that can be used in the current inventions is shown in FIG. 2 in publication of PMID: 28071970. The antibody or bispecific antibody or multi-specific antibody to be used in the current inventions can also be based on nanobody or other antibody mimetics such as affibody. Additional examples of antibody mimetics scaffold can be used include Affibody, Affilin, Anticalin, Atrimer, Avimer, bicyclic peptide, cys-knots, DARPin, FN3 (Adnectin), Fynomers, Kunitz domain and Obodies.

The monospecific antibody or bispecific antibody or multi-specific antibody can also have a tandem format of Fc as previously described (e.g. those in FIGS. 1-3). FIG. 5 shows examples of monospecific antibody or bispecific antibody having tandem format of Fc. As shown in FIG. 5a, a second pair of Fab (through their N terminal) is added to the second Fc's C terminal via a peptide linker, preferably the flexible peptide linker described previously. The resulting antibody has two pair of Fab and two Fc. The two pair of Fab can be the same to generate a mono specific antibody. In some embodiments, the two pairs of Fab target two epitopes on the same antigen, which will generate higher specificity and affinity. In some embodiments, the two pairs of Fab target two antigens, which is essentially a bispecific antibody, for example one pair target tumor cell surface antigen and another pair target surface antigen of antigen presenting cell; in another example, one pair target tumor cell surface antigen and another pair target surface antigen of cytotoxic T cell. Any pair or both pairs of the Fab can be replaced with a scFv or nanobody or other antibody mimetic. FIG. 5b shows an exemplary format of a bispecific antibody having both Fab and scFv. FIG. 5c shows another exemplary format having two pair of Fab and two Fc where the two pairs of Fab are connected in tandem. FIG. 5d shows another exemplary format having two pair of Fab and three Fc. More repeating Fc can also be incorporated similarly.

Similarly, the monospecific antibody or bispecific antibody or multi-specific antibody can also have repeat CH2 as previously described (e.g. those in FIG. 3). FIG. 6 shows examples of monospecific antibody or bispecific antibody having repeat CH2. As shown in FIG. 6a-d, a second pair of Fab (through their N terminal) is added to the CH2 or CH3's C terminal via a peptide linker, preferably the flexible peptide linker described previously. The expression will have two pair of Fab and multiple CH2. The two pairs of Fab can be the same to generate a monospecific antibody. In some embodiments, the two pairs of Fab target two epitopes on the same antigen, which will generate higher specificity and affinity. In some embodiments, the two pairs of Fab target two antigens, which is essentially a bispecific antibody. Any pair or both pairs of the Fab can be replaced with a scFv or nanobody or other antibody mimetic. FIGS. 6e and 6g shows an exemplary format of a bispecific antibody having both Fab and scFv. FIG. 6f shows another exemplary format having two pair of Fab and CH2 repeat where the two pairs of Fab are connected in tandem. More repeating CH2 can also be incorporated similarly.

The linker and linker 2 in the antibody (e.g. those in FIGS. 5 and 6) can be a longer than those shown in cited publications to allow better binding at multiple target simultaneously. For example, it can be a flexible hydrophilic peptide having 20-100 AA, such as (GaSb)n where in a, d and n are integers between 1-10. In some embodiments, the flexible peptide linker is a G/A/D/E rich peptide. Additional flexible liker and hydrophilic peptide linker can be those described previously in the current application used to connect the repeated CH2 or CH2CH3 in the antibody.

The multi-specific antibody used in the current inventions can also be replaced by a multi-specific affinity ligand, wherein the affinity ligand contains non-antibody structure/moiety having binding affinity such as antibody mimetics and aptamer. In some preferred embodiments, similar to bispecific antibody, those multi-specific affinity ligand has one or more arm (molecule) that can bind with tumor cell surface antigen (tumor cell surface marker), while the other arm/arms (molecule) can bind with surface antigen (cell surface marker) of antigen presenting cells. In some embodiments, one arm is antibody (or antibody fragment, which is used exchangeably with antibody in the current inventions), another arm is antibody mimetics and aptamer. In some embodiments, all arms are non-antibody ligand such as antibody mimetics or aptamer. Therefore, they have a general structure of
APC binding molecule-optional linker-tumor cell surface binding molecule conjugate

The tumor cell surface binding molecule can be replaced with the cell surface anchoring molecule disclosed in the current invention such as a lipid compound, therefore, they have a general structure of:
APC binding molecule-optional linker-cell surface anchoring molecule conjugate

The APC binding molecule is essentially a phagocytosis enhancing molecule. These APC binding molecule-optional linker-tumor cell surface binding molecule conjugate, APC binding molecule-optional linker-cell surface anchoring molecule conjugate and affinity ligand that can bind with tumor cell surface antigen (e.g. antibody mimetic, aptamer) can be further conjugated with an immune activity enhancing agent (e.g. TLR agonist or STING agonist) to be used in the formulations of the current invention to replace said antibody or im combination with said antibody.

For example, antigen presenting enhancing molecule can be affinity ligand (e.g. antibody, antibody fragment, antibody mimetics, aptamer) for antigen presenting cells (e.g. their cell surface marker). Examples of antigen presenting cells include dendritic cells, macrophages, B cells, T cells and NK cells. For example, antigen presenting enhancing molecule can be antibody against DC cell surface marker, e.g. antibody (or its fragment) against CD11C or affinity ligand for macrophage such as Fab against CD14. Other examples include affinity ligand for pattern recognition receptors (PRRs) such as the Toll-like receptor (TLRs), which are widely expressed on APC surface. Additional DC cell surface markers are disclosed throughout the current inventions.

These antibodies described above including native or engineered antibody and multi-specific antibody can also be conjugated with or an immune activity enhancing agent (e.g. STING agonist or TLR agonist including TLR agonist peptide) to form antibody drug conjugates. These antibody drug conjugates can be given systematically or being injected intratumorally or injected into or proximal to the tumor draining lymph node or applied to the site where tumor is removed during surgery to treat cancer. Said antibody drug conjugate can be incorporated into a sustained release formulation such as those in situ gelling system with optional other immune activity enhancing agent similar to other type of cancer cell inactivating agent to treat cancer.

Examples of TLR agonist and STING agonist are disclosed previously in the current application. Additional examples of TLR agonist and STING agonist to conjugate with antibody or other affinity ligand as well as the protocol to prepare conjugate can be found in US patent application U.S. Ser. No. 16/140,309, U.S. Ser. No. 12/280,472, U.S. Ser. No. 16/817,864, U.S. Ser. No. 15/333,285, U.S. Ser. No. 16/940,697, U.S. Ser. No. 16/711,652, U515/110,685, U.S. Ser. No. 14/408,268, U.S. Ser. No. 16/476,640 and U.S. 62/753,175 as well as journal publications such as publication of PMID: 26133029. Those TLR agonist or STING agonist can be conjugated to the antibodies described in the current inventions using well-known protocol such as those described in above prior arts by replacing the antibody in these publications with the antibody in the current applications. The conjugation and the protocol can be either site specific or non-site specific such as those described in publications of PMID: 24423619, PMID: 30973187 and PMID: 25981886.

FIGS. 3,5 and 6 in publication of PMID: 2442361911 showed examples of the conjugate, chemistry and conjugate from site specific and non-site specific conjugation. The conjugate (ADC) normally has a general structure of antibody-attachment site-linker-drug formula. Examples of the drug can be TLR agonist and STING agonist. Examples of general linkers include NHS ester reacting with amine of antibody to form amide bond linkage, isothiocyanate reacting with amine of antibody to form thiourea bond linkage, haloacetyl reacting with —SH of antibody to form thioether bond linkage and maleimide reacting with —SH of antibody to form thioether bond linkage.

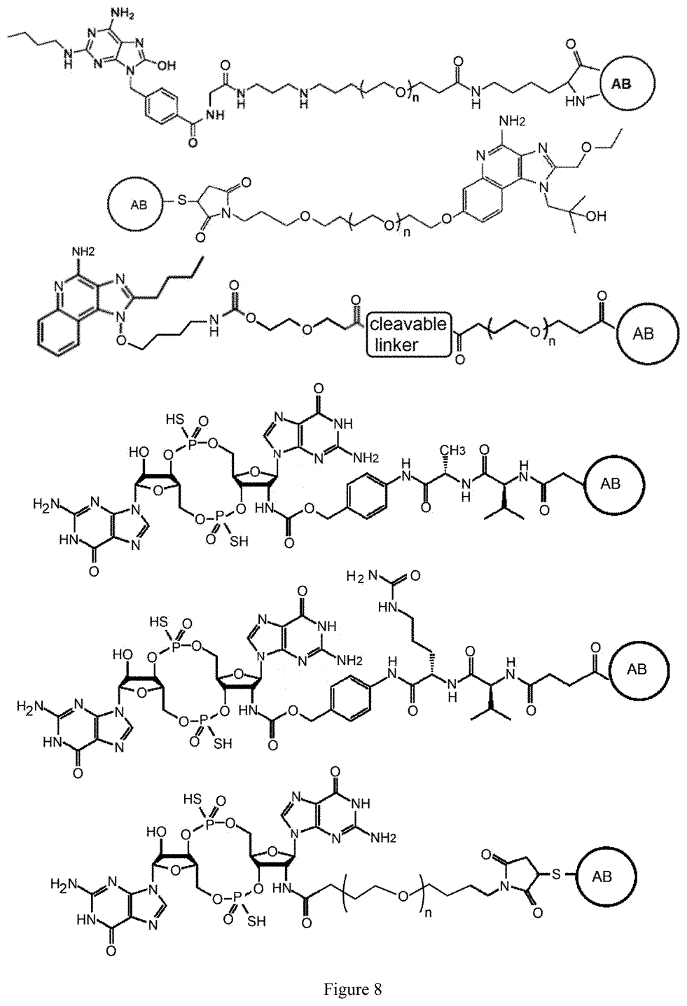

FIGS. 7 and 8 show examples of the TLR agonist or STING agonist moiety in the conjugate of the current inventions. FIGS. 7 and 8 shows examples of the TLR agonist moiety or STING agonist moiety with linker in the conjugate of the current inventions, n is an integer between 0-10, multiple TLR agonist or STING agonist moiety can be present in one ADC. Cleavable linker such as a peptide sequence of MMP-9 cleavage site as described in FIG. 17 of U.S. patent application Ser. No. 16/924,184 can be incorporated as shown in FIG. 8.

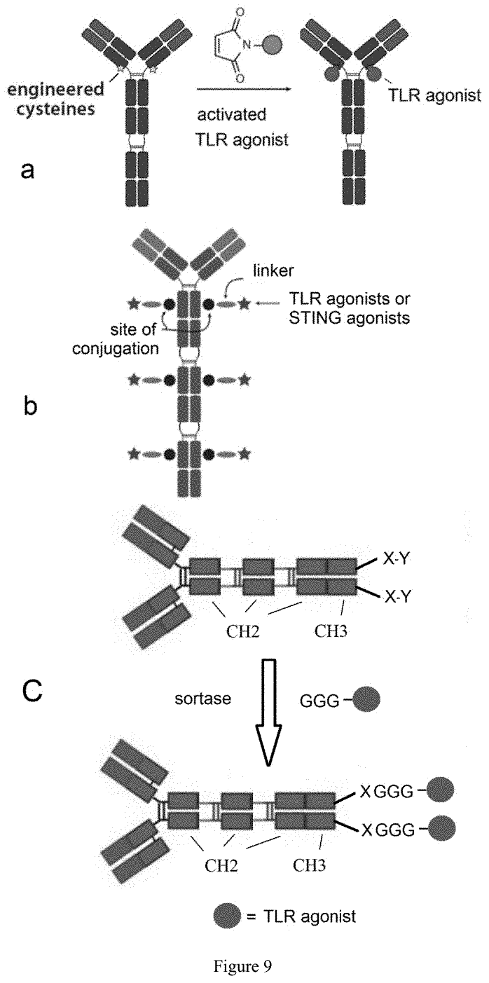

Fc engineered antibody and antibody having tandem Fc or repeat CH2 can be conjugated with immune enhancing agent readily and used in the current inventions. FIG. 9 shows examples of TLR agonist or STING agonist conjugated to antibodies having tandem Fc or repeat CH2. In FIG. 9a an antibody having 2 Fc as shown in FIG. 1 is engineered to have a cysteines on their Fab, which can react with a activated TLR agonist having maleimide group to from an antibody-TLR conjugate. In FIG. 9b an antibody having 2 Fc as shown in FIG. 1 is conjugated with 6 TLR agonists or STING agonists at their CH2 domain using glycotransferases, the protocol of using glycotransferase to perform site specific conjugation can be found in related publications and ready adopted for the current invention. In FIG. 9c an antibody having 3 CH2 domains as shown of FIG. 3 is conjugated with 2 TLR agonists or STING agonists at their C-terminal using sortase based site specific conjugation by attaching a sortase recognition motif peptide at its C-terminal, wherein X-Y is a sortase recognition peptide sequence.

Bispecific antibody or multi-specific antibody can also be conjugated with immune enhancing agent and used in the current inventions. FIG. 10 shows examples of the TLR agonist conjugated to bispecific antibody of the current inventions in different format. In FIG. 10a a bispecific antibody of FIG. 5b having tandem Fc is conjugated with 4 TLR agonists or STING agonists at its Fab/scFV's C-terminal using sortase based site specific conjugation. In FIG. 10b a bispecific antibody of FIG. 6a having CH2 repeat is conjugated with 2 TLR agonists at CH3 region. In FIG. 10c a di-scFV type bispecific antibody is conjugated with a TLR agonist at its C terminal, which bind with a tumor cell surface antigen (e.g. EpCAM) and DC cell surface antigen (e.g. DC-SIGN). In FIG. 10d a knobs into holes type bispecific antibody is conjugated with TLR agonist at its CH3 domain. In FIG. 10e a dual variable domain bispecific antibody is conjugated with TLR agonist. In FIG. 10f a half-life extended BiTE (HLE-BiTE) type bispecific antibody is conjugated with TLR agonist at its C terminal, TLR agonist or STING agonist can also be conjugated to other position of the HLE-BiTE using suitable site specific conjugation strategy. Other type of bispecific antibody or multi-specific antibody can also be conjugated with immune enhancing agent listed in the current invention readily.

The tumor surface antigen targeting antibody including bispecific and multi-specific antibody, antibody derivatives and antibody drug conjugate described in the current invention can be incorporated into the sustained release formulation such as in-situ gelling system or implant to be injected intratumorally or injected into or proximal to the tumor draining lymph node or applied to the site where tumor is removed during surgery to treat cancer. They can also be used as a stand-alone therapy to be used systematically, e.g. by IV injection at therapeutically effective amount. When the TLR agonist in the ADC is a TLR peptide agonist, fusion protein can be constructed instead of chemical conjugation. The TLR peptide can be fused to the antibody by genetic engineering and expression, e.g. at the N or C terminal of the antibody such those on their heavy chain or light chain with optional linker sequence. The fusion protein contains an antibody (including antibody fragment) moiety targeting tumor antigen and a peptide TLR agonist moiety. Examples of TLR peptide agonist that can be used can be found in patent application U.S. Ser. No. 15/432,180, U.S. Ser. No. 14/377,782, U.S. Ser. No. 15/557,647, U.S. Ser. No. 15/557,649 and their citations.

The TLR peptide agonist in the fusion protein can also be replaced with other peptide, protein including antibody that can activate/boost the function of immune system and immune cell, which are described previously. For example, it can be an antibody-granulocyte macrophage colony-stimulating factor fusion, an antibody-Interferon gamma fusion, an antibody-TNF alpha fusion, an antibody-TNF beta fusion, an antibody-immunity boosting interleukin (including their derivatives and mimics) fusion such as an antibody-IL2 fusion, an antibody-IL12 fusion, an antibody-IL3 fusion, an antibody-IL7 fusion, an antibody-IL15 fusion, an antibody-super antigen (e.g. SEA) fusion, an antibody-interleukin superagonist (e.g. interleukin-15 superagonist) fusion, an tumor targeting antibody-immunity boosting receptor targeting antibody (e.g. anti CD40, anti OX40, anti CD28) fusion which is essentially a multi specific antibody; wherein the non-tumor binding moiety can be fused to the tumor targeting antibody moiety by genetic engineering and expression, e.g. at the N or C terminal of the tumor targeting antibody such those on their heavy chain or light chain with optional linker sequence. In one example, the fusion is a anti HER2 antibody-IL15 fusion. In another example, the fusion is a anti HER2 antibody-IL15 super agonist (e.g. N-803) fusion. In another example, the fusion is a anti EGFR antibody-SEA fusion. In another example, the fusion is a anti PD-L1 antibody-IL2 fusion. In some embodiments, the tumor targeting antibody can be replaced with immune cell (e.g. APC or T cell) targeting antibody. The antibody in these ADC and fusion can also be replaced with antibody mimetic or none-antibody type affinity ligand.

More than one unit of antigen (e.g. mimotope or alpha-gal or L-rhamnose or their derivatives) or affinity ligand for antibody or affinity ligand for APC surface marker, more than one type of antigen (e.g. mimotope) or affinity ligand for antibody or affinity ligand for APC surface marker and more than one unit of cell surface anchoring molecule such as cholesterylamine can be incorporated in the conjugate. For example, they can also be conjugated to a soluble polymer backbone either linear or branched or the combination of linear and branched (e.g. dextran, peptide, poly acrylic acid or the like) as shown in FIG. 11. Insoluble polymer back bone can also be used, which is essentially a nano or micro particle. The cell membrane/surface anchoring molecule can also be molecule other than cholesterylamine, such as lipid molecule and cell membrane anchoring peptide. Example of the lipid molecule suitable for the current invention are described in prior U.S. patent application Ser. No. 15/945,741, 16/271,877 and 16/924,184. Other cell membrane anchoring lipid molecules can also be used. Furthermore, one or more immune activity enhancing agent such as TLR agonist or STING agonist or their combination can also be incorporated in the conjugate. An example of conjugate containing multiple TLR agonist, antigen and cell surface anchoring lipid is also shown in FIG. 11b.

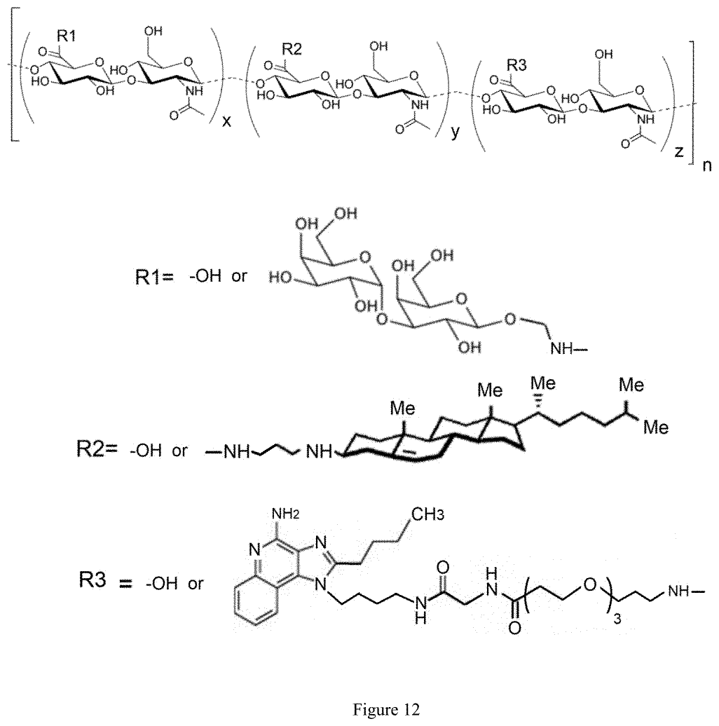

FIG. 12 shows examples of conjugate using hyaluronic acid (HA) based backbone. x, y, z are integers from 0-10, n is integer from 2-10000. The antigen, lipid and TLR agonist are conjugated to the HA's —COOH group forming amide bond linkage. The conjugate can be synthesized by react HA activated NHS ester (reaction product of HA+EDAC+NHS) with mixture of antigen, lipid and TLR agonist having amine terminus. R1 in FIG. 12 can also be Herceptin mimotope peptide such as that shown in FIG. 1 of U.S. patent application Ser. No. 16/271,877. R2 in FIG. 12 can also lipid other than 3β-cholesterylamine such as fatty lipid, alkylamine (e.g. CH₃(CH₂)₉—NH—) or other cholesterol derivatives. R3 shown in FIG. 12 can also be other TLR agonist or STING agonist.

In some embodiments, one conjugate of TLR agonist, lipid and antigen contains no more than 3 copies of TLR agonist moiety, no more than 3 copies of antigen moiety and no more than 3 copies of lipid moiety. In some embodiments, said conjugate contains no more than 2 copies of TLR agonist moiety, no more than 2 copies of antigen moiety and no more than 2 copies of lipid moiety. In some embodiments, said conjugate contains 1 copy of TLR agonist moiety, 1 copy of antigen moiety and 1 copy of lipid moiety. The TLR agonist can be replaced with STING agonist as well.

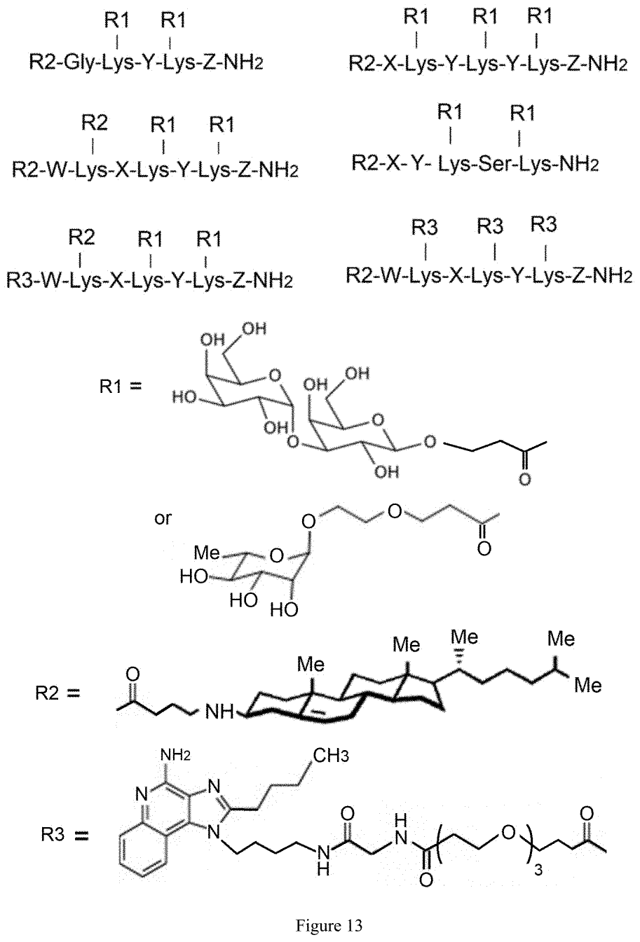

The backbone can also be peptide in linear or branched form. The peptide contains multiple amino acid having reactive side chain (e.g. Lys, Cys, Glu, Asp) to conjugate with antigen, affinity ligand for APC, TLR/STING agonist and cell surface anchoring agent. The amino acid in the peptide can be D amino acid to avoid enzyme degradation. Other peptidomimetic such as peptoid can also be used in the peptide backbone. FIG. 13 shows examples of conjugate using lysine containing peptide as backbone. W is affinity ligand for APC such as those described previously. X, Y and Z are spacers, which can be peptide analogue or peptide containing 1-10 amino acids. For example, X, Y and Z can be a single amino acid independently selected from Ala, Gly and Ser, or dipeptide consisting of Ala, Gly and Ser. In some embodiments, spacer is optional. FIG. 13 show different exemplary formats of the conjugate and additional format can be adopted readily using peptide or peptoid as backbone. Lipid, TLR agonist and antigen can be conjugated at either the N terminal of the peptide or the side chains. Exemplary R1 can be α-gal or α-Rha as shown in in FIG. 13 or antibody mimotope peptide such as those disclosed in U.S. patent application Ser. No. 16/271,877. R2 in FIG. 13 can also lipid other than 3β-cholesterylamine such as fatty acid, e.g. CH₃(CH₂)₉—CO— or other cholesterol derivatives. R3 shown in FIG. 13 can also be other TLR agonist or STING agonist. R1, R2 and R2 can be conjugated with the N terminus of amino acid or amine sidechain of lysine to form amide linkage.

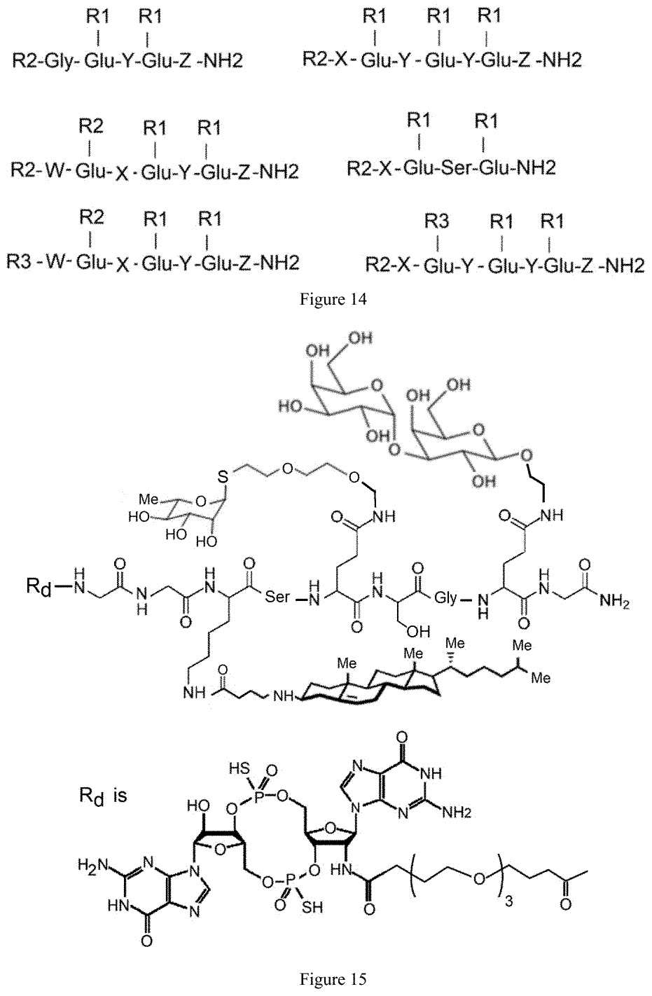

FIG. 14 shows examples of conjugate using Glu containing peptide as backbone. W is affinity ligand for APC such as those described previously. X, Y and Z are spacers, which can be peptide analogue or peptide containing 1-10 amino acids. For example, X, Y and Z can be a single amino acid independently selected from Ala, Gly and Ser, or dipeptide consisting of Ala, Gly and Ser. In some embodiments, spacer is optional. FIG. 14 show different formats of the conjugate and additional format can be adopted readily using peptide as backbone. Lipid, TLR agonist, affinity ligand for APC and antigen can be conjugated at either the N terminal of the peptide or the side chains. Exemplary R1 can be the α-gal derivatives as shown in FIGS. 12 and 13 or antibody mimotope peptide such as those disclosed in U.S. patent application Ser. No. 16/271,877. R2 can be the 3β-cholesterylamine derivatives in FIGS. 12 and 13 or other lipid such as fatty acid, e.g. CH₃(CH₂)₉—CO—, or other cholesterol derivatives. R3 can be same TLT agonist in FIGS. 12 and 13 or other TLR agonist or STING agonist. R1, R2 and R3 in FIG. 13 can be conjugated with the N terminus of amino acid to form amide linkage. R1, R2 and R3 in FIG. 12 can be conjugated with the —COOH sidechain of Glu to form amide linkage.

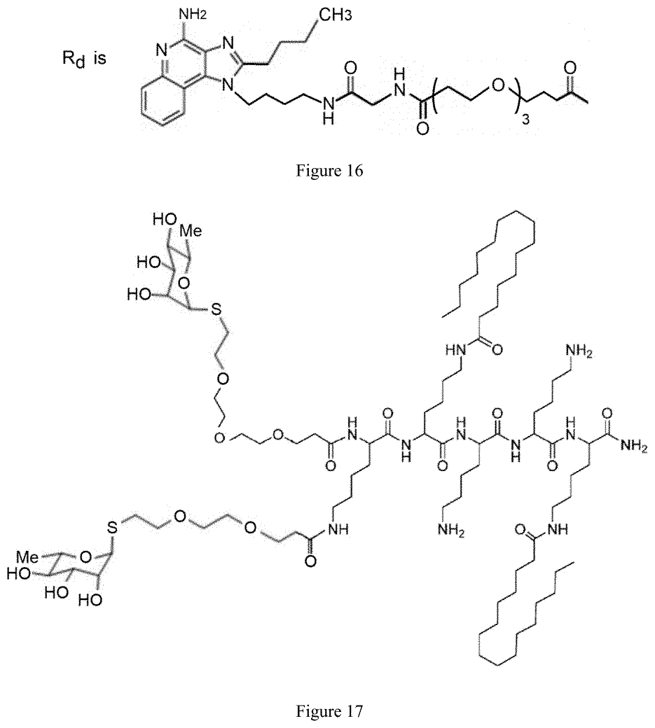

FIG. 15 show an example of conjugate using both Lys and Glu sidechain to conjugate lipid and two different antigens. It also contains a STING agonist (Rd in the figure) conjugated to N terminus of glycine. It contains two different antigens. One antigen is a N-substituted rhamnose mimic and another antigen is α-gal. FIG. 16 shows an example of TLR agonist (Rd in the figure) that can replace the STING agonist in the conjugate using Lys/Glu in FIG. 15 and the resulting conjugate contains a TLR agonist instead of a STING agonist. The cholesterol amine lipid in these examples can also be replaced with other type of lipid molecules such as those described in prior applications, e.g. fatty acid, phosphor lipid or cholesterol, and multiple lipid molecules can be incorporated in the conjugate by varying the peptide sequence.

FIG. 17 shows an example of conjugate using Lys containing peptide as backbone and two fatty acid as lipid. Shorter chain fatty acid (e.g. 6-12 carbon) and unsaturated fatty acid (8-20 carbon, 1-3 double bond, such as myristoleic acid) can be used instead in the conjugate and the two fatty acids can be different.

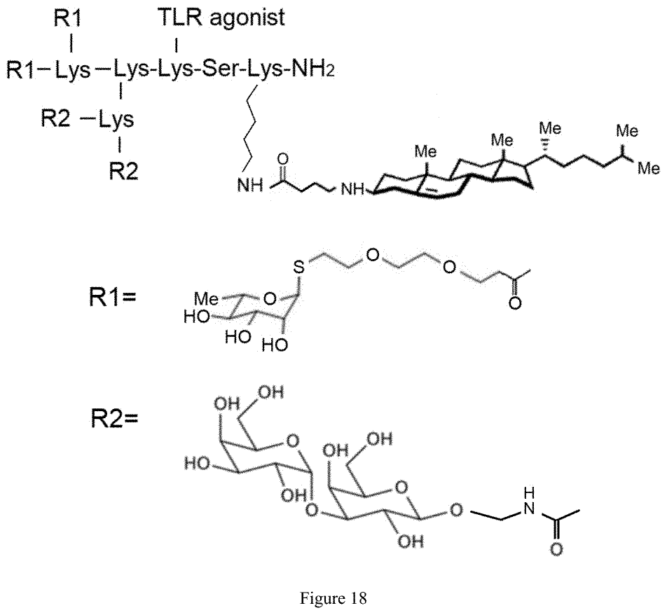

FIG. 18 shows an example of conjugate having 4 antigens, two S-substituted rhamnose mimic and two antigen is α-gal. The two S-substituted rhamnose can be replaced with the native rhamnose. The four antigens can also be the same, either 4 rhamnose mimic or 4 α-gal. Exemplary TLR agonist can be those TLR agonist in FIG. 16.

In some embodiments, the formulation suitable for the current invention contains one or more antibody type drug and immune activity enhancing agent at therapeutically effective amount. Therefore, the current invention discloses composition and formulations containing antibody against tumor cell surface antigen and immune activity enhancing agent (e.g. TLR agonist or STING agonist) in a sustained release formulation such as in-situ gelling system or implant to be injected intratumorally to treat cancer or injected into or proximal to the tumor draining lymph node to treat cancer or applied to the site where tumor is surgically removed to treat cancer.

In certain embodiments, formulations can be a solution containing about 10-50 mg/mL antibody against tumor cell surface antigen (e.g. Herceptin, cetuximab, antibody against EpCAM) and 1-5 mg/mL imiquimod suspension and/or 2-10 mg/mL poly IC in 3-9% sodium alginate in combination with another pharmaceutical acceptable excipient. For example, the antibody against EpCAM can be IgG, IgM, Fc engineered antibody as previously described, antibody having tandem Fc or CH2 repeat (e.g. those in FIGS. 1-4), bispecific antibody or antibody drug conjugate described in the current applications. The antibodies can be mixed with other component (e.g. solution of 1-5 mg/mL imiquimod suspension and/or 2-10 mg/mL poly IC in 3-9% sodium alginate) right before injection to generate said formulation, therefore allowing the user to use commercially available antibodies. The user can use the formulation solution containing an immune function enhancing agent with optional sialidase as diluents to reconstitute lyophilized antibodies; or use antibody solutions as diluents to reconstitute the lyophilized formulation containing an immune function enhancing agent and optional sialidase.

In example 1, the composition/formulation is a solution of pH 5-8 containing 20˜50 mg/mL trastuzumab, 1 mg/mL ADU-S100, 2 mg/mL imiquimod, 4 mg/mL poly IC, optional 5 mg/mL α-GalCer and optional 2 mg/mL neuraminidase (human) in 5% w/w sodium alginate or 20% Poloxamer 407. It will be injected into the HER2 positive tumor at 100˜500 μL/cm³ tumor volume to treat cancer every 10 days for total 3 times. Check point inhibitor can be given to the patient at the same time and later. Alternatively, the formulation contains 20˜100 mg/mL trastuzumab or Fc engineered IgG against HER2 having higher ADCP activity, L-arginine, L-cysteine and L-tryptophan at 20˜100 mg/mL, Celecoxib 20 mg/mL, curcumin or BHT 20 mg/mL, cyclophosphamide 10 mg/mL, 3 mg/mL ADU-S100, 2 mg/mL imiquimod, 2 mg/mL poly IC, 5 mg/mL α-GalCer and optional 2 mg/mL neuraminidase, 5% sodium alginate in 0.2×PBS to have a final pH of 5-8. Suitable amount of hyaluronic acid is added to the solution to reach a viscosity of 500,000-5,000,000 cps. It can be injected into the Her2 positive tumor at 0.1˜0.5 mL/cm³ tumor volume to treat cancer every 10 days for total 3 times. Check point inhibitor can be given to the patient at the same time and later. Herceptin can be replaced with antibody against other tumor cell surface antigen expressed on the target tumor such as antibody against PD-L1 for PD-L1 positive tumor, antibody against EpCAM for EpCAM positive tumor, antibody against EGFR for EGFR positive tumor. The antibody can be IgG, IgM, Fc engineered antibody as previously described, antibody having tandem Fc or CH2 repeat (e.g. those in FIGS. 1-4) as well as bispecific antibody or antibody drug conjugate described in the current applications.

In example 2, the formulation contains 20˜100 mg/mL humanized IgG1 antibody against EpCAM having 3Fc as shown in in FIG. 1, 2 mg/mL imiquimod, 2 mg/mL poly IC, 5 mg/mL ADU-S100, L-arginine, L-cysteine and L-tryptophan at 20˜100 mg/mL, poly aspirin 20 mg/mL, tadalafil 3 mg/mL, nitro-aspirin 5 mg/mL, all-trans retinoic acid 5 mg/mL, 5 mg/mL α-GalCer, gemcitabine 10 mg/mL, cucurbitacin 10 mg/mL, and optional 2 mg/mL neuraminidase in 6% sodium alginate or in or 20% Poloxamer 407. It can be injected into the tumor at 100˜500 μL/cm³ tumor volume to treat cancer every 10 days for total 3 times. Alternatively, the formulation contains 50 mg/mL trastuzumab, 50 mg/mL humanized IgM against EGFR or humanized IgG1 antibody against EGFR having CH2 repeat as shown in format a in FIG. 3, 0.5 mg/mL ADU-S100, 1 mg/mL imiquimod or 0.2 mg/mL 3M-052, 3 mg/mL poly IC, optional 3 mg/mL SD-101 and optional 2 mg/mL neuraminidase in a self-gelling system such as 5% sodium alginate or 20% Poloxamer 407. In another example, the formulation contains 50 mg/mL trastuzumab emtansine or ADC against HER2 having the format of those in FIG. 9, 20 mg/mL PLGA nanoparticle encapsulating 10% imiquimod, and 10 mg/mL poly IC in a self-gelling system such as 5% sodium alginate or 20% Poloxamer 407.

In example 3, the formulation contains 50 mg/mL monospecific antibody against EGFR, or bispecific antibody against EGFR and MHC II of APC, 0.5-5 mg/mL ADU-S100, 2-5 mg/mL imiquimod and optional 2 mg/mL neuraminidase (Vibrio cholera) and optional solubility enhancing excipient such as 0.1% tween-20 or 5% propylene glycol in 50 mg/mL sodium alginate or 20% Poloxamer 407. The mono or bispecific antibody can be in any format shown in FIGS. 5-6.

In example 4, the formulation contains 50 mg/mL monospecific antibody against EGFR having the format of FIG. 5a, 20 mg/mL PLGA nano or micro particle encapsulating 10% imiquimod and 5% SB 11285, 2 mg/mL antibody against OX40, 2 mg/mL poly IC, 50 μg/mL granulocyte-monocyte colony-stimulating factor, 1×10⁴-1×10⁵ U/mL of IFN-α or IFN-gamma, 1-10 MIU/mL IL-2 in a self-gelling system such as 5% (50 mg/mL) sodium alginate. These anti EGFR antibody containing formulations can be injected into EGFR-expressing tumor at 100˜500 μL/cm³ tumor or 0.5 mL-5 mL/tumor to treat cancer every 10 days for total 3 times.

In example 5, the composition is a liquid containing 50 mg/mL anti HER2 IgG-TLR conjugate having 3 Fc as shown in FIG. 9b and 1-5 mg/mL STING agonist such as ADU-S100 or MK-1454 or SB 11285 in a sustained released system such as 3-6% sodium alginate or 20% Poloxamer 407. The antibody type drug can be mixed with other ingredients in the composition right before injection, therefore allow the user to use the commercially available antibody type drug. For example, the user can use the formulation solution containing vaccine adjuvant with commercially available antibody drug solution as diluents to reconstitute the lyophilized formulation containing vaccine adjuvant and sialidase.

Similarly, chemotherapy drug can also be used as cancer cell inactivating agent in the current invention. Example of these drugs include alkylating agents, antimetabolites, anti-microtubule agents, topoisomerase inhibitors, cytotoxic antibiotics and cisplatin family drugs such as those disclosed in embodiments type 3.

The current invention and prior applications disclose antibody binding molecule-optional linker-cell surface anchoring molecule conjugate and its use to treat cancer. The main propose of the antibody binding molecule moiety is to increase antigen presenting for tumor associated antigen, which is mainly from the Fc moiety of the antibody introduced by the said antibody binding molecule. Similarly, besides Fc moiety introduced by antibody binding molecule, other molecule (or can be called as moiety) that can increase cancer cell antigen presenting can also be used to build the conjugate instead of the antibody binding molecule. The general structure of the conjugate is antigen presenting enhancing molecule-optional linker-cell surface anchoring molecule conjugate and its use can be similar to the use of the above antibody binding molecule-optional linker-cell surface anchoring molecule conjugate, e.g. by replacing the antibody binding molecule-optional linker-cell surface anchoring molecule conjugate in the above examples and embodiments with antigen presenting enhancing molecule-optional linker-cell surface anchoring molecule conjugate. For example, antigen presenting enhancing molecule can be affinity ligand (e.g. antibody, antibody fragment, antibody mimetics, aptamer) for antigen presenting cells (e.g. their cell surface marker). Examples of antigen presenting cells and antigen presenting enhancing molecule are disclosed previously in the current application.

One of the main type of antigen presenting enhancing molecule is the molecule that can enhance endocytosis. Therefore, the conjugate that can be used in the current invention has the structure of endocytosis enhancing molecule-optional linker-cell surface anchoring molecule conjugate. Endocytosis pathways can be subdivided into four categories: namely, receptor-mediated endocytosis (also known as clathrin-mediated endocytosis), caveolae, macropinocytosis, and phagocytosis. In some embodiments, the conjugate that can be used in the current invention has the structure of phagocytosis enhancing molecule-optional linker-cell surface anchoring molecule conjugate as disclosed in U.S. patent application Ser. Nos. 16/271,877 and 16/924,184, which also disclosed phagocytosis inducing molecules such as PAMPS, opsonins and intracellular molecules presented on the cell surface. Therefore these molecules can also be used as phagocytosis enhancing molecule to construct the conjugate or used by its self as phagocytosis enhancing molecule (be incorporated in the formulation of the current invention). Examples of the phagocytosis enhancing molecule are disclosed in U.S. patent application Ser. Nos. 16/271,877 and 16/924,184. They can be conjugated to the cell surface anchoring molecule to form antigen presenting enhancing molecule-optional linker-cell surface anchoring molecule conjugate, which can be injected intratumorally in combination with said immune activity enhancing agent to form in situ vaccine against tumor. If they themselves have affinity to cells or can be deposited around the cell after injection, the conjugation may not be required, examples include C1q, C3b, C4, C3-convertase and secreted Pattern recognition receptors (PRRs), which can be injected into the tumor directly without conjugation.

In some embodiments, the conjugate is Toll-like receptor (TLR) ligand-optional linker-cell surface anchoring molecule conjugate or C-type lectin (CLEC) ligand-optional linker-cell surface anchoring molecule conjugate including Mannose-binding lectin (MBL) ligand-optional linker-cell surface anchoring molecule conjugate. More than one unit of TLR/MBL/CLEC ligand and more than one unit of cell surface anchoring molecule can be incorporated in a conjugate. Examples of TLR/CLEC/MBL ligand include bacterial carbohydrates (such as lipopolysaccharide or LPS, mannose, pathogen cell wall peptidoglycan), nucleic acids (such as bacterial or viral DNA or RNA), bacterial peptides (flagellin, microtubule elongation factors), peptidoglycans and lipoteichoic acids (from gram-positive bacteria), N-formylmethionine, lipoproteins, fungal glucans and chitin, synthetic TLR ligands such as imidazoquinoline, CpG ODNs and poly IC. Examples of these conjugate include CpG ODN-fatty acid conjugate, CpG ODN-cholesterylamine conjugate and those disclosed in U.S. patent application Ser. Nos. 16/271,877 and 16/924,184. In some embodiments, the structure of the conjugate is pathogen-associated carbohydrate-optional linker-cell surface anchoring molecule conjugate or pathogen cell wall peptidoglycan-optional linker-cell surface anchoring molecule conjugate, where in the pathogen-associated carbohydrate can be selected from N-acetyl-D-glucosamine, N-acetylmuramic acid, D-mannose, N-acetyl-mannosamine, L-fucose, N-acetylglucosamine (GlcNAc) and N-acetylmuramic acid (MurNAc) disaccharide, and the cell surface anchoring molecule can be lipid molecule such as cholesterylamine. Other cell surface anchoring molecule and other endocytosis enhancing molecule can also be used to make the conjugate. The resulting conjugate can be either used alone or in combination with other antibody binding molecule-optional linker-cell surface anchoring molecule conjugate as intratumoral injection.

In one example, the formulation contains 2˜50 mg/mL Clq-lipid conjugate or C3b or C3-convertase, 0.1-1 mg/mL ADU-S100, 2 mg/mL imiquimod, 2 mg/mL poly IC, 5 mg/mL α-GalCer and 2 mg/mL neuraminidase (human) in a self-gelling system such as 3-5% (30-50 mg/mL) sodium alginate. It can be injected into the tumor at 0.05-0.5 mL/cm³ tumor volume or 0.5 ml-5 ml/tumor to treat cancer every 10 days for total 3 times. In another example, the formulation contains 2-10 mg/mL ADU-S100, 10˜100 mg/mL CpG ODN-cholesterylamine conjugate or poly IC-cholesterylamine conjugate, 5 mg/mL α-GalCer and 2 mg/mL neuraminidase (human) in a self-gelling system such as 3-5% (30-50 mg/mL) sodium alginate.

In example 6, the formulation is a solution containing 100˜200 mg/mL Trastuzumab mimotope-lipid conjugate, CpG ODN 2216-fatty acid conjugate, 0.5-5 mg/mL ADU-S100, 1-10 mg/mL imiquimod, 2 mg/mL poly IC, 10 mg/mL antibody against CD25, 10 mg/mL antibody against OX40, 50 μg/mL granulocyte-monocyte colony-stimulating factor, 1×10⁴-1×10⁵ U/mL of IFN-α and/or IFN-gamma, 1-10 MIU/mL IL-2 in 5-9% sodium alginate. Optional solubility enhancing excipient such as 0.1% tween-20 or 5% propylene glycol can also be incorporated in the formulation. After the patient receive the intratumoral injection with the above formulation, the patient is intravenously injected with Ipilimumab 3˜10 mg/kg every 3 weeks for 4 doses, or Atezolizumab 1200 mg IV q3wk until disease progression. It can also be injected into or proximal to the tumor draining lymph node Herceptin 5-10 mg/kg can also be intravenously injected before or after the intratumoral injection of the above formulation.

In one example, the formulation contains 10˜100 mg/mL imiquimod-cholesterylamine conjugate or CpG ODN-folate conjugate, 2 mg/mL ADU-S100, 5 mg/mL α-GalCer and 2 mg/mL neuraminidase-lipid conjugate, 0.5% hyaluronic acid in 3-6% sodium alginate or in 20% Poloxamer 407. In another example, the formulation contains 10˜100 mg/mL CpG ODN-cell membrane anchoring peptide conjugate or C3 convertase-lipid conjugate, 1 mg/mL ADU-S100, 5 mg/mL α-GalCer, 1% HPMC in 3-6% sodium alginate or in 20% Poloxamer 407. They can be injected into the tumor at 100˜500 uL/cm3 tumor volume or 0.5 ml-3 ml/tumor to treat cancer every two weeks for total 3 times. Check point inhibitor can be given to the patient at the same time and later.

Optionally nonsteroidal anti-inflammatory drugs (NSAIDs) and their suitable form as those disclosed in U.S. patent application Ser. Nos. 16/271,877 and 16/924,184 can be added to the composition/formulation of the current inventions and prior applications to be injected intratumorally or injected into or proximal to the tumor draining lymph node or applied to the site where tumor is removed during surgery to treat cancer. Suitable amount can be between 0.01˜5% w/w.

Optionally metformin or disulfiram or diethyldithiocarbamate or CD39 inhibitor or CD73 inhibitor or adenosine receptors (A2A and A2B) inhibitor or EP4 receptor inhibitor or their combinations can be added to the composition/formulation of the current inventions and prior applications to be injected intratumorally or injected into or proximal to the tumor draining lymph node or applied to the site where tumor is removed during surgery to treat cancer. Suitable amount can be between 0.01˜5% w/w. In one example it is 1% w/w. They can be in form of active molecule, prodrug (e.g. their ethyl ester), liposome, emulsion, micelle, insoluble precipitate (e.g. in complex with condensing agent), conjugated to polymer drug carrier (e.g. dextran), encapsulated in implant, coated on or encapsulated in biodegradable micro particle/nano particle (e.g. those made of biodegradable polymer such as PLA, PLGA, PCL, PGA or PHB) or in a self-gelling system. Suitable size of the particle can be between 10 nm˜100 um.

Optionally glucocorticoid can be added to the composition/formulation of the current inventions and those in prior applications to be injected intratumorally or injected into or proximal to the tumor draining lymph node or applied to the site where tumor is removed during surgery to treat cancer. Suitable amount can be between 0.01˜5% w/w. Examples of them are cortisone, prednisone, prednisolone, methylprednisolone, dexamethasone, betamethasone, triamcinolone, fludrocortisone, deoxycorticosterone, aldosterone and beclometasone. They can be in form of active molecule, prodrug (e.g. their ethyl ester), liposome, emulsion, micelle, insoluble precipitate (e.g. in complex with condensing agent), conjugated to polymer drug carrier (e.g. dextran), encapsulated in implant, coated on or encapsulated in biodegradable micro particle/nano particle (e.g. those made of biodegradable polymer such as PLA, PLGA, PCL, PGA or PHB) or in a self-gelling system. Suitable size of the particle can be between 10 nm˜100 um.

Optionally L-arginine or L-cysteine or L-tryptophan or free radical scavenger/antioxidant or agent that can inactivate Treg and/or inhibit tumor-associated macrophage as those disclosed in U.S. patent application Ser. Nos. 16/271,877 and 16/924,184 or their combinations can be added to the composition/formulation of the current inventions and those in prior applications to be injected intratumorally or injected into or proximal to the tumor draining lymph node or applied to the site where tumor is removed during surgery to treat cancer. Suitable amount can be between 0.01˜5% w/w. They can be in form of active molecule, prodrug (e.g. their ethyl ester), liposome, emulsion, micelle, insoluble precipitate (e.g. in complex with condensing agent), conjugated to polymer drug carrier (e.g. dextran), encapsulated in implant, coated on or encapsulated in biodegradable micro particle/nano particle (e.g. those made of biodegradable polymer such as PLA, PLGA, PCL, PGA or PHB) or in a self-gelling system. Suitable size of the particle can be between 10 nm˜100 um. Enzyme inhibitor that can prevent Arg/Cys/Tyr depletion can also be used, e.g. indoleamine 2,3-dioxygenase inhibitor that can block the depletion of tryptophan in the tumor.

Optionally agent that can inactivate MDSC (Myeloid-derived suppressor cell) and their suitable form as those disclosed in U.S. patent application Ser. Nos. 16/271,877 and 16/924,184 can be added to the composition/formulation of the current inventions and those in prior applications to be injected intratumorally or injected into or proximal to the tumor draining lymph node or applied to the site where tumor is removed during surgery to treat cancer.

The injection formulation can also be a thermal phase changing formulation. Thermal phase changing formulation is a formulation that change its phase from liquid at low temperature or room temperature (25° C.) to semisolid/gel when temperature increases to body temperature (37° C.)., which can use Poloxamer as excipient. A thermal phase changing injectable formulation containing both the conjugate or cancer killing microbes and immune enhancing agent such as TLR agonist can be injected intratumorally to treat cancer. The preparation of this kind of thermal phase changing injectable formulation can be adopted from related publications readily by the skilled in the art.

In one example, a solution containing 20˜200 mg/mL L-rhamnose-cholesterylamine conjugate of U.S. patent application Ser. No. 15/945,741, 1 mg/mL ADU-S100, 3 mg/mL poly IC or 3 mg CpG ODN 2216 or both, 20 mg/mL biodegradable PLGA micro or nano particles encapsulating 20% imiquimod, and granulocyte-monocyte colony-stimulating factor (10-200 μg/mL), L-arginine, L-cysteine and L-tryptophan at 20˜100 mg/mL, poly aspirin 20 mg/mL, glutathione or SOD 5 mg/mL, N-hydroxy-L-Arginine 10 mg/mL, tadalafil 3 mg/mL, axitinib 10 mg/mL, nitro-aspirin 5 mg/mL, all-trans retinoic acid 5 mg/mL, 5 mg/mL α-GalCer, gemcitabine 10 mg/mL, cucurbitacin 10 mg/mL with optional 3-6% sodium alginate is prepared. Suitable amount of surfactant can be added to from stable suspension. Suitable amount of carbomer is added to the solution to reach a viscosity of 1,000,000-5,000,000 cps. After the patient receive the intratumoral injection with the above formulation at 0.05-0.25 mL/cm³ tumor volume, the patient is intravenously injected with ipilimumab 3-10 mg/kg every 3 weeks for 4 doses, or atezolizumab 1200 mg IV q3wk until disease progression.

In some embodiments the cancer cell inactivating agent can be cancer cell killing microbe as disclosed in U.S. patent application Ser. Nos. 15/945,741, 16/271,877 and 16/924,184 or be the combination of cancer cell killing microbe with other cancer cell inactivating agent such as the antigen-lipid conjugate. The formulations and compositions in the disclosure of U.S. patent application Ser. Nos. 16/271,877 and 16/924,184 as well as those in current application that contains the cancer cell killing microbes can be either injected intratumorally to treat cancer or injected into or proximal to the tumor draining lymph node to treat cancer or applied to the tumor removed site during surgery to treat cancer. The current invention also discloses a method to treat cancer by apply cancer cell killing microbe to the site where tumor is removed during surgery. The cancer cell killing microbe can be either a non-sustained release form solution such as those used for IV injection or in a sustained release form such as an in-situ gelling formulation or implant. The implant can be either a hydro gel or a dried form in the shape of film, membrane or the like described previously. In some embodiments, a composition and formulation to treat cancer comprise therapeutically effective amount of said cancer cell killing microbes (e.g. bacterial or virus) in a sustained release formulation including self-gelling system such as 3-9% alginate (e.g. sodium alginate) or 15˜25% poloxamer. Additional gelling enhancing agent including polymer such as hyaluronic acid, HPMC, chitosan, CMC (e.g. 0.5%˜2.5% w/w) can also be incorporated in the formulation.

If lyophilized form is desired, additional, bulking agent/lyoprotectant (e.g. 1˜10% sucrose, mannitol, trehalose) can also be included. The composition and resulting formulation can be applied to the site where tumor is removed during surgery at therapeutically effective dose, e.g. 5˜50% of the dose used for IV injection of same microbe. Additional calcium salt (e.g. 2% CaCl₂ solution) can be applied to the site too to form gel quickly. Alternatively, calcium salt can be added to said self-gelling formulation containing cancer cell killing microbe to form a hydro gel and the hydro gel is applied to the site where tumor is removed during surgery as an implant. The implant can also be dried to remove water partially or completely to form a film and the film is applied to the site where tumor is removed during surgery as an implant to treat cancer. The non-cancer cell killing microbe cancer cell inactivating agent (e.g. antigen-lipid conjugate, chemotherapy drug, antibody) in the compositions/formulations described in U.S. patent application Ser. Nos. 15/945,741, 16/271,877, 16/924,184 and current application can be replaced with therapeutically effective amount of cancer cell killing microbe instead. Alternatively, cancer cell killing microbe can be added to these compositions/formulations resulting in combo compositions/formulations. Examples of cancer cell killing microbes can be either cancer cell killing bacterial or cancer cell killing virus (oncolytic virus) or cancer killing parasites or cancer killing fungi or any microbe that can kill cancer cells or their combination. They can be either given systematically or injected intratumorally. Examples of cancer cell killing bacterial include engineered Salmonella typhimurium, Clostridium novyi-NT spores, Clostridium sporogenes, Coley's toxins, BCG and others such as those disclosed in U.S. patent application Ser. Nos. 16/271,877 and 16/924,184. Example dose of cancer cell killing bacterial in a sustained release formulation used for intratumoral injection can be between 1,000,000˜10,000,000,000 copies for each tumor. Examples of cancer cell killing virus (oncolytic virus) include oncolytic poxvirus, JX-594, Imlygic (T-VEC), enterovirus RIGVIR, oncolytic adenovirus (H101), Cavatak, oncolytic virus M1, CG0070 and Reolysin. Additional examples can be found in U.S. patent application Ser. Nos. 16/271,877 and 16/924,18. Example dose of cancer cell killing virus in a sustained release formulation used for intratumoral injection can be between 105˜10¹⁵ pfu for each tumor, e.g. 1×10¹⁰ pfu JX-594 can be injected into a tumor.

In one example, a solution containing 10\hat{ }8 CFU of Salmonella typhimurium/mL, 1 mg/mL ADU-S100 or 3 mg/mL poly IC or 3 mg CpG ODN 2216 or their combination, 20 mg/mL biodegradable PLGA nano particles encapsulating 20% imiquimod and 3-9% sodium alginate is prepared. Optionally suitable amount of linear or cross linked hyaluronic acid is added to the solution as a viscosity enhancer to reach a viscosity of 5,000,000 cps. The patient receives the intratumoral injection with the above formulation at 0.05˜0.25 mL/cm³ tumor volume. In another example, a solution containing 10\hat{ }8 CFU of Salmonella typhimurium/mL, 1 mg/mL ADU-S100 or 3 mg/mL poly IC or 3 mg CpG ODN 2216 or their combination, 20 mg/mL biodegradable PLGA nano particles encapsulating 20% imiquimod, 4% sodium alginate is prepared. Optionally suitable amount of linear or cross linked hyaluronic acid is added to the solution as a viscosity enhancer to reach a viscosity of 1,000,000-5,000,000 cps. The patient receives the intratumoral injection with the above formulation at 0.1-0.25 mL/cm³ tumor volume.

In another example, a solution containing 1-10 million C. novyi-NT/mL, optional 2 mg/mL SB 11285, 3 mg/mL poly IC or 3 mg CpG ODN 2216 or both, 20 mg/mL biodegradable PLGA nano particles encapsulating 20% imiquimod, L-arginine, L-cysteine and L-tryptophan at 20˜100 mg/mL, poly aspirin 20 mg/mL, glutathione or SOD 5 mg/mL, N-hydroxy-L-arginine 10 mg/mL, tadalafil 3 mg/mL, axitinib 10 mg/mL, nitro-aspirin 5 mg/mL, all-trans retinoic acid 5 mg/mL, 5 mg/mL α-GalCer, gemcitabine 10 mg/mL, cucurbitacin 10 mg/mL, 3-9% sodium alginate is prepared. Optionally suitable amount of HPMC is added to the solution to reach a viscosity of 500,000-1,000,000 cps. The patient receives the intratumoral injection with the above formulation at 0.5 mL/cm³ tumor volume. In another example, a solution containing 1×10\hat{ }9 pfu JX-594/mL, optional 5 mg/mL SB 11285, 3 mg/mL poly IC or 3 mg CpG ODN 2216 or both, 20 mg/mL biodegradable PLGA nano particles encapsulating 20% imiquimod, 3-9% sodium alginate is prepared. Optionally suitable amount of hyaluronic acid is added to the solution to reach a viscosity of 100,000-500,000 cps. After the patient receive the intratumoral injection with the above formulation at 0.2-2 mL/tumor, the patient is intravenously injected with ipilimumab 3-10 mg/kg every 3 weeks for 4 doses, or atezolizumab 1200 mg IV q3wk until disease progression.

In another example, a solution containing 1×10\hat{ }12 pfu oncolytic virus M1/mL, 3 mg/mL poly IC or 3 mg CpG ODN 2216 or both, 20 mg/mL biodegradable PLGA nano particles encapsulating 20% imiquimod and 10% MK-1454, L-arginine, L-cysteine and L-tryptophan at 20˜100 mg/mL, poly aspirin 20 mg/mL, glutathione or SOD 5 mg/mL, N-hydroxy-L-arginine 10 mg/mL, tadalafil 3 mg/mL, axitinib 10 mg/mL, nitro-aspirin 5 mg/mL, all-trans retinoic acid 5 mg/mL, 5 mg/mL α-GalCer, gemcitabine 10 mg/mL, cucurbitacin 10 mg/mL, 3.5% sodium alginate is prepared. Optionally suitable amount of hyaluronic acid is added to the solution to reach a viscosity of 500,000 cps. After the patient receive the intratumoral injection with the above formulation at 0.5 mL-3 mL/tumor, the patient is intravenously injected with Ipilimumab 3-10 mg/kg every 3 weeks for 4 doses, or Atezolizumab 1200 mg IV q3wk until disease progression. In another example, the formulation is a solution containing 1×10\hat{ }10 pfu imlygic/mL, 10 mg/mL CpG ODN 2216-fatty acid conjugate, 5 mg/mL MK-1454, 10 mg/mL imiquimod, 2 mg/mL poly IC, 10 mg/mL antibody against CD25, 10 mg/mL antibody against OX40, 1×10⁴-1×10⁵ U/mL of IFN-α, 1×10⁴-1×10⁵ U/mL IFN gamma, 1-10 MIU/mL IL-2, 6% sodium alginate. The patient receives the intratumoral injection with the above formulation. Additional chemotherapy drug such as 1% w/w paclitaxel can also be added to the above formualtions.

In some embodiments, the cell surface anchoring antigen conjugate of the current invention has the following formula:

native antigen-optional linker-cell surface anchoring molecule

as those disclosed in U.S. patent application Ser. Nos. 15/945,741, 16/271,877 and 16/924,184, which also disclosed examples of this kind of conjugates and they can be readily used in current application, such as α-gal lipid conjugates, L-rhamnose lipid conjugates and DNP-lipid conjugates. FIG. 3 of Ser. No. 15/945,741 application shows examples of 3β-cholesterylamine, 3β-cholesterylamine containing moiety and their derivatives or analogues used for the conjugate.

As described in U.S. patent application Ser. No. 15/945,741 and 16/924,184, the cell surface anchoring antigen conjugate described herein can comprise more than one antigen, which can be either more than one of the same or a combination of different antigens, and optional linker or spacer can be used to connect the antigen to the cell surface anchoring molecule. More than one unit of native antigen, more than one type of native antigens and more than one unit of cell surface anchoring molecule. They can be either in monomer or oligomer format within the conjugate. They can also be conjugated to a soluble polymer backbone (e.g. dextran, poly peptide, poly acrylic acid or the like). The conjugate can further comprise a cancer cell binding domain (e.g. RGD peptide, folic acid) to increase its targeting to a cancer cell, as described in U.S. patent application Ser. No. 15/945,741, which will allow intravenous (IV) injection instead of intratumoral injection,

The current invention provides compositions and sustained release formulations for use as in situ cancer vaccines to promote a strong immune response against cancer cells. In one embodiment, provided is a pharmaceutical composition comprising a cell surface anchoring antigen conjugate and an immune function enhancing agent. Examples of suitable immune function enhancing agent can be found in U.S. patent application Ser. Nos. 15/945,741, 16/271,877 and 16/924,184, which include pattern recognition receptor (PRR) ligands, RIG-I-Like receptor (RLR) ligands (e.g. RIG-I agonist, MDA5 agonist, LGP2 agonist), Nod-Like receptor (NLR) ligands, C-Type Lectin Receptors (CLR) ligands, STING agonist, and Toll-like receptor agonist, or a combination thereof. The immune function enhancing agent can be a vaccine adjuvant. Preferably the Toll-like receptor ligand is a Toll-like receptor (TLR) agonist. Exemplary Toll-like receptor (TLR) agonists can be used are listed in U.S. patent application Ser. Nos. 15/945,741 and 16/271,87. Examples of STING agonists that can be used for the current invention can be found but not limited to the STING agonists disclosed in U.S. patent application Ser. No. 16/924,184. For these formulations, the cell surface anchoring antigen conjugate can be any of those described herein above, or any known in the art.

Also provided is a composition comprising a cell surface anchoring antigen conjugate as described herein and an immune function enhancing agent. Such compositions can be injected into the tumor (e.g., at 10 μL to about 200 μL/cm³ tumor volume). The immune function enhancing agents can be administered as a prodrug, liposome, emulsion, micelle, sustained release formulation, insoluble precipitate (e.g. in complex with condensing reagent), conjugated to polymer drug carrier (e.g. dextran) or encapsulated in biodegradable micro particle/nano particle (e.g. those made of biodegradable polymer such as PLA, PLGA, PCL, PGA or PHB).

U.S. application Ser. No. 16/924,184 disclose novel STING agonist and their use for cancer treatment. These STING agonists are conjugated with a lipid moiety, therefore become a long lasting (long in vivo half-life) STNG agonist. Conjugating a lipid moiety to STING agonist can increase their local retain once being intratumorally injected therefore show longer local half-life, and lower sider effect and higher efficacy. The conjugate is essentially a novel structure of STING agonist, either in an active agonist form or a prodrug form. The conjugate comprises a STING receptor binding moiety and a lipid moiety. The current invention also disclose a method to extend the half-life of STING agonist by conjugating a STING receptor binding moiety and a lipid moiety. The current invention and U.S. application Ser. No. 16/924,184 disclose a novel structure of STING agonist, which comprise a STING receptor binding moiety and a half-life extending moiety (e.g. a PEG moiety or a lipid moiety or a cell membrane anchoring moiety such as cell membrane anchoring peptide). Examples of the conjugate are shown in FIGS. 13, 14 and 15 of U.S. application Ser. No. 16/924,184. As previously above, the current invention also disclose a method to increase STING agonist's half-life in tumor by conjugating a STING agonist with a half-life extender (e.g. PEG or a lipid moiety). For example, fatty acid or alkyl phosphoric acid or PEG with a carboxylic acid/phosphoric acid end can be conjugated to the —OH group/groups of the STING agonist (e.g. the —OH on the sugar of CDN type STING agonist) to form a cleavable ester linkage. Long alkyl chain alcohol or cholesterol can be conjugated to the phosphate group/groups of the STING agonist to form a cleavable phosphate ester linkage. Furthermore, the above method and strategy can also be applied to TLR agonist by replacing the STING agonist moiety with TLR agonist moiety in the conjugate described above. Examples of fatty acid and cholesterol derivative conjugated TLR7/8 agonist are shown in FIG. 17 of U.S. patent application Ser. No. 16/924,184.

Example 7: In an aqueous media, the formulation contains 1-100 mg/mL cancer cell lysing agent (e.g. α-gal-cholesterylamine or L-rhamnose-cholesterylamine conjugate or their mixture at 1:1 molar ratio or any conjugate described in U.S. patent application Ser. No. 15/945,741 or the conjugate described in FIGS. 11-18), optional 0.1-50 mg/mL STING agonist such as ADU-S100 or MK-1454 or SB 11285, 0.1-50 mg/mL TLR7/8 agonist (e.g. imiquimod or gardiquimod or resiquimod), 0.1-50 mg/mL TLR3/RLR agonist (e.g. poly IC or polyICLC), 0.1-50 mg/mL TLR9 agonist (e.g. CpG ODNs such as ODN 1826 or ODN 2216) and optional 0.1-50 mg/mL neuraminidase from Vibrio cholera in 1×PBS or in 3-9% sodium alginate or in 20% Poloxamer 407. It can further contain 5% sucrose and be lyophilized to give the solid formulation. In one embodiment, it contains 30 mg/mL cancer cell lysing agent (e.g. α-gal-cholesterylamine or L-rhamnose-cholesterylamine conjugate), 5 mg/mL imiquimod, 5 mg/mL poly IC, 5 mg/mL class A CpG ODN 2216 and optional 5 mg/mL neuraminidase in 5% sodium alginate or 20% Pluronic F127. The solution or the solid dosage reconstituted with water can be injected to the tumor at 50 μL˜300 μL/cm³ tumor size or 200 uL-2 mL/tumor to treat cancer.

Example 8: In an aqueous media, the composition contains 100 mg/mL cancer cell lysing agent (e.g. α-gal-cholesterylamine or L-rhamnose-cholesterylamine conjugate), optional 0.5 mg/mL ADU-S100, 2 mg/mL imiquimod, 2 mg/mL poly IC, 2 mg/mL class A CpG ODN 2216 or class B CpG ODN and 2 mg/mL neuraminidase-lipid conjugate in 5% sodium alginate and 10% mineral oil to form an emulsion. The drugs are in active form, one or more or all of them can also be in the form of prodrug, liposome, micelle, insoluble precipitate (e.g. in complex with condensing reagent), conjugated to polymer drug carrier (e.g. dextran) or coated on or encapsulated in biodegradable micro particle/nano particle as previously described. For example, compounds having one or more amine groups that can precipitate poly IC and CpG ODN therefore generate insoluble precipitates can be used as sustained release drug formulations. Examples of co-precipitation compound are described previously. Surfactant can be added to the precipitates to from stable suspension.

Example 9: Encapsulation of poly IC or CpG ODN or ADU-S100 in biodegradable micro or nano sphere can be performed according to published protocol such as that example 3 of disclosed in U.S. patent application Ser. No. 16/924,184. The prepared micro/nanosphere can be used as an immune function enhancing agent. It can be mixed with suitable amount of in situ gelling agent described in the current application such as 5% sodium alginate or 20% Pluronic F127, and optional bulking agent/lyoprotectant if lyophilized form is preferred. Other cell lysing agent such as antigen-lipid conjugate or chemotherapy drug can also be encapsulated within the particle. It can be used either alone to be injected into tumor, or mixed together with other cell lysing agent (which can also be encapsulated within different particle) such as solution of antibody against tumor (e.g. Herceptin) on site right before injected into the tumor as a in situ gelling formulation.

Example 10: The nanosphere encapsulating poly IC and imiquimod and SB 11285 can be prepared using a double emulsion water/oil/water system according to published protocol such as that example 4 of disclosed in U.S. patent application Ser. No. 16/924,184. PBS is used for the wash solutions and the final resuspension media contains 3-9% sodium alginate or 20% poloxamer 407. The suspension is stored at −20° C. The in situ gelling suspension can be injected to tumor alone to treat tumor or mix together with other agents and then to be injected into the tumor or injected into or proximal to the tumor draining lymph node.

Example 11: A solution contains 20˜200 mg/mL of any conjugate described in FIGS. 11-18, 3 mg/mL poly IC or 3 mg CpG ODN 2216 or both, optional 20 mg/mL biodegradable PLGA nano particles encapsulating 20% imiquimod and optional 5% MK-1454, and granulocyte-monocyte colony-stimulating factor (10-200 μg/mL), 5% sodium alginate. 20% Poloxamer 407 can be used instead of 5% sodium alginate. Suitable amount of surfactant can be added to from stable suspension. After the patient receive the intratumoral injection with the above formulation at 0.1-0.3 mL/cm³ tumor volume, the patient is intravenously injected with Ipilimumab 3-10 mg/kg every 3 weeks for 4 doses, or atezolizumab 1200 mg IV every 3 weeks until disease progression.

Example 12: A solution contains 50˜100 mg/mL of a conjugate selected from those described in FIG. 11-18 with optionally 100 mg/mL α-gal-cholesterylamine conjugate, optional 5 mg/mL ADU-S100, 10 mg/mL imiquimod, 5 mg/mL poly IC, 5 mg/mL CpG ODN 2216, 100 μg/mL granulocyte-monocyte colony-stimulating factor, 1×10⁴-1×10⁵ U/mL of IFN-α or IFN-gamma or both, 1-10 MIU/mL IL-2 and 5% sodium alginate or 20% Poloxamer 407. After the patient receive the intratumoral injection with the above formulation, the patient is intravenously injected with ipilimumab 3-10 mg/kg every 3 weeks for 4 doses, or atezolizumab 1200 mg IV q3wk until disease progression.

Example 13: A solution contains 100˜200 mg/mL PLGA nano particles encapsulating 20% a conjugate selected from those in FIG. 11-18, 1 mg/mL ADU-S100, 4 mg/mL poly IC, 4 mg/mL CpG ODN 2216, 2 mg/mL imiquimod or 0.1 mg/mL R848, optional 0.5-2 mg/mL α-GalCer, optional 25×10⁴ U/mL of IFN-α, 5 MIU/mL IL-2. The patient will receive the intratumoral injection with the above formulation at 0.5-1 mL/tumor. The composition can further comprises 5% sodium alginate and the patient will receive the intratumoral injection with the above formulation at 1-3 mL/tumor.

Example 14: A solution contains 100˜200 mg/mL PLGA micro or nano particles encapsulating 20% a conjugate selected from those in FIG. 11-18, optional 5 mg/mL ADU-S100, 5 mg/mL poly IC, 5 mg/mL CpG ODN 2216, 0.5 mg 3M-052, optional 5 MIU/mL IL-2 and optional 5% sodium alginate or 20% Poloxamer 407. After the patient receive the intratumoral injection with the non-alginate containing formulation at 0.1-0.25 mL/cm³ tumor volume or alginate containing formulation at 0.2-0.5 mL/cm³ tumor volume, the patient is intravenously injected with ipilimumab 3-10 mg/kg every 3 weeks for 4 doses, or atezolizumab 1200 mg IV q3wk until disease progression.

Example 15: A formulation contains 10˜50 mg/mL bispecific antibody against HER2 and a APC surface antigen described in the current application, optional 2 mg/mL ADU-S100, 2 mg/mL imiquimod, 5 mg/mL poly IC, optional 5 mg/mL α-GalCer and optional 2 mg/mL neuraminidase (human) in 1×PBS. It is injected into the Her2 positive tumor at 0.2-0.5 mL/tumor to treat cancer every 10 days for total 3 times. Check point inhibitor can be given to the patient at the same time and later. Alternatively, the formulation contains 20˜100 2330 mg/mL IgG against HER2 having 2 FC or 3 CH2 as shown in FIGS. 1-4, optional 3 mg/mL ADU-S100, 6 mg/mL imiquimod, 5 mg/mL poly IC, optional 5 mg/mL α-GalCer and optional 2 mg/mL neuraminidase in 5% sodium alginate or in 20% poloxamer. It is injected into the Her2 positive tumor at 500 uL-2 mL/tumor to treat cancer every 3 weeks for total 3 times. Check point inhibitor can be given to the patient at the same time and later.

Example 16: A formulation contains 50 mg/mL IgG1 against HER2 having a tandem Fc with optional 50 mg/mL cetuximab, 2 mg/mL imiquimod, 1 mg/mL ADU-S100, and optional 1 mg/mL neuraminidase in 5% sodium alginate. Alternatively, the formulation contains 50 mg/mL antibody-TLR agonist conjugate against HER2 having a format selected from those in FIGS. 7-10, 20 mg/mL PLGA nanoparticle containing 10% imiquimod and 2 mg/mL poly IC, 1% HPMC in 5% sodium alginate or in 20% poloxamer.

Example 17: A formulation containing 50 mg/mL cetuximab having an additional Fc repeat, 1-3 mg/mL ADU-S100, 1-3 mg/mL poly IC, 2-5 mg/mL imiquimod or 0.2-0.5 mg/mL R848 and optional 1 mg/mL neuraminidase in 5% sodium alginate. Alternatively, the formulation contains 50 mg/mL cetuximab having an additional Fc repeat, 20 mg/mL PLGA nanoparticle containing 10% imiquimod, 2 mg/mL poly IC, optional 50 μg/mL granulocyte-monocyte colony-stimulating factor, optional 1×10⁴-1×10⁵ U/mL of IFN-α or IFN gamma, optional 1-10 MIU/mL IL-2 in 5% sodium alginate or in 20% poloxamer. These cetuximab containing formulations can be injected into EGFR-expressing tumor at 0.1-0.3 mL/cm³ tumor volume to treat cancer every 2 weeks for total 3 times.

In many of the embodiments and examples disclosed in this application, cell surface anchoring conjugate is incorporated. A modification of these embodiments and examples is to remove the cell surface anchoring conjugate from these compositions and formulations while the other components are still the same. Therefore, those cell surface anchoring conjugate is optional in these embodiments and examples. They can be used the same to treat cancer.

As used herein, a liquid containing insoluble substance such as particles such as microparticles or nano particle, aggregate, precipitate is still considered a solution. For example, some TLR agonists such as imiquimod have low solubility in water and exist in aqueous solution as insoluble form such as crystal. A liquid containing solid state imiquimod is still considered a solution and the solid form drug will have a sustained drug release effect.

The agents, compositions and formulations in the current inventions are to be injected intratumorally to treat cancer or injected into or proximal to the tumor draining lymph node to treat cancer or applied to the tumor removed site during surgery to treat cancer. The route of administration of them can be also be used in combination. For example, a drug loaded in situ gelling formulation can be used as intratumoral injection a few weeks before tumor removal surgery and then applied to the tumor removed site during surgery and then injected into or proximal to the tumor draining lymph node a few days after surgery. In another example, a drug loaded in situ gelling formulation can be used as intratumoral injection and also be injected into or proximal to the tumor draining lymph node during the same treatment period, e.g. same day. Different route of administration can also use different formulations. For example, in some embodiments the formulation to be injected into or proximal to the tumor draining lymph node can be a sustained release formulation containing immune function enhancing agent only without tumor cell inactivating agent, e.g. an in situ gelling formulation containing TLR agonist only as those described in the current inventions.

### Embodiments Type 1

The current invention also discloses a composition and its use to treat cancer, which is to be mixed with cancer treating therapeutic antibody including ADC to form a sustained release formulation such as self-gelling formulation and then said formulation is injected into the tumor or injected into or proximal to the tumor draining lymph node to treat cancer. Said formulation can also be applied directly to the open wound area where tumor is removed right after surgery to treat cancer and then added with optional Ca′ salt solution to form gel in situ, or it can be further mixed with suitable amount of Ca′ salt to form a gel and then apply the gel directly to the open surgery area where tumor is removed. In some embodiments, the composition is a liquid containing suitable amount of self-gelling polymers disclosed in the current invention or its lyophilized form with optional bulking agent/lyoprotectant added before lyophilization, the amount of said polymer need to be enough to form gel in vivo after it is mixed with antibody drug. In some embodiments, the composition and the formulation is 2˜50% sodium alginate in water or saline and the pH is adjusted to 5-8 with the addition of concentrated base or acid such as NaOH or HCl. In some embodiments, the composition and the formulation is 2˜20% sodium alginate in water or saline and the pH is 5-8 with the addition of 2M NaOH or 2M HCl. In some embodiments the formulation's osmolality is adjusted with physiological acceptable excipient to have a osmolality similar to physiological condition. In some embodiments the formulation has low osmolality and low pH buffering capacity so it will not affect the osmolality and pH value of the antibody drug containing formulation after being mixed together especially for those solid type (e.g. lyophilized form) preformulated antibody drug product; for example, the formulation has osmolality and pH buffering capacity lower than 0.5×PBS. Calcium salt or other divalent cationic salt can be incorporated in the formulation at a low concertation that will not cause gelling in vitro. For example, 2-20 mg of Ca′ per 1 g of alginate can be used. The low contraction of Ca′ salt e.g. 0.05%˜0.5% calcium gluconate in the formulation, will help the gelling in vivo. The formulation can further comprise gelling enhancing polymers previously disclosed such as 0.1˜1% HA, CMC, HPMC, carbomer, MC, chitosan; 10-30% poloxamer or their combinations. Preferably the final solution will have 2˜6% alginate after mixing said composition/formulation with antibody drug and preferably the antibody drug will be 1˜20% w/w in the final mixture, which can be done by adjusting the ratio between said composition/formulation and antibody drug as well as the original alginate concertation in the formulation. The composition/formulation can also be lyophilized and reconstituted with water or antibody solution before use. A bulking agent/lyoprotectant can be added such as 5% sucrose before lyophilization, which will also improve the dissolution speed of alginate. In one example, the formulation is 9% sodium alginate solution in saline at pH 6-7. The reconstituted Herceptin solution containing 21 mg/mL of trastuzumab prepared according its reconstitution method in its product insert is mixed with 9% sodium alginate at 1:1 v/v ratio to generate the final solution to be injected intratumoral (e.g. 0.1 ml-0.5 mL/cm³ tumor volume) to HER2+ tumor. It can also be applied to the surgical area where tumor is removed, optionally with sequential addition of Ca′ salt solution to promote gelling in situ as previously described. If the antibody drug to be mixed is a solid dose, e.g. Kanjinti (trastuzumab-anns) for injection, low tonicity and lower alginate formulation can be used. For example, the formulation is 3.5% sodium alginate solution in water having a pH value of 6-7, 20 ml of it will be added to a 420 mg vial of Kanjinti to generate a 21 mg/mL trastuzumab-anns solution containing ˜3.5% sodium alginate to treat cancer as intratumoral injection or applied to the area where tumor is removed. Different concertation of trastuzumab-anns solution can be made accordingly to provide a final solution having physiologically acceptable tonicity and pH and desired alginate concentration (e.g. 2%˜6%), by adjusting the tonicity, volume added, alginate concentration of the said formulation to be added. For example 40 mL of a formulation containing 3.5% sodium alginate solution in 0.45% NaCl having a pH value of 6 can be added to a 420 mg vial of KANJINTI (solid form) to generate a 10.5 mg/mL trastuzumab-anns solution containing ˜3.5% sodium alginate, which remain a physiological osmolality. In another example, the formulation is 2.5% sodium alginate, 0.5% linear HA (MW 1-10M Da) or cross-linked HA in water having a pH value of 6-7; 10 ml of it is added to a Enhertu (trastuzumab deruxtecan) 100 mg vial to obtain a final concentration of 10 mg/mL antibody for HER2+ tumor treatment.

Instead of Herceptin/Kanjinti/Enhertu, other antibody anti-cancer drugs; such as sacituzumab govitecan (Trop-2-directed antibody), belantamab mafodotin (antibody against the B-cell maturation antigen), Padcev (enfortumab vedotin for the treatment of cancer expressing nectin-4), Polivy (polatuzumab vedotin against CD79B), moxetumomab pasudotox (an anti-CD22 immunotoxin), Besponsa (inotuzumab ozogamicin, a CD22-directed antibody drug conjugate), brentuximab vedotin (Adcetris, targets tumor cells expressing the CD30 antigen), Kadcyla (trastuzumab emtansine, a HER2-targeted antibody), durvalumab (Imfinzi, an anti PD-L1 antibody), Tecentriq (atezolizumab, an anti-programmed death-ligand 1 monoclonal antibody), Bavencio (avelumab, an antibody against PD-L1), catumaxomab, rituximab, Bexxar, cetuximab, bevacizumab, panitumumab, pertuzumab, Kadcyla and other check point inhibitor antibody drugs; can also be mixed with said alginate containing formulations to generate a solution containing antibody (e.g. 1-20 mg/mL) and alginate (e.g. 2-9% w/w) or poloxamer (e.g. 20% Poloxamer 407) to treat cancer similarly.

In some embodiments, besides or instead of mixing with antibody said formulation is pre mixed or further mixed with therapeutically effective amount of other protein drugs that has anti-cancer effect such as those proteins that can activate/boost the function of immune system and immune cell (including APC, B cell and T cells) described previously in the current invention, e.g. granulocyte macrophage colony-stimulating factor, FLT3L, IFN-α, IFNγ, anti-tumor interleukin such as interleukin-2, or their derivatives such as PEGylated derivative. In one example, the formulation is 5% sodium alginate, 2% HPMC in water having a pH value of 6-7; 10 ml of it is mixed with 10 mL Bavencio 20 mg/mL concentrate, and further mixed with 0.2-0.5 mL Proleukin (18 million international units (1.1 mg) Proleukin/mL) to form the final solution to be injected into the tumor or injected into or proximal to the tumor draining lymph node to treat cancer. Alternatively interleukin-2 can also be preloaded in the 5% sodium alginate with 2% HPMC, or in the 20% Poloxamer 407, as a ready to use formulation, which will be mixed with antibody later for treatment.

In some embodiments, therapeutically effective amount of TLR agonist (e.g. poly IC or imiquimod or R848 or 3M-052 or CPGODN such as SD-101 or their combinations) or STING agonist or their combinations can be further incorporated into the said alginate containing composition/formulation described above as a ready to use formulation, which can be used as a standalone drug or be mixed with antibody or other protein drug against cancer to form the final solution to be injected into the tumor or injected into or proximal to the tumor draining lymph node to treat cancer or applied to the tumor removed area, similar to those described above. The formulation is a solution containing alginate as in situ gelling agent, TLR agonist or STING agonist or their combination, optional gelling enhancing agent and optional protein drug against cancer; or its lyophilized form with optional bulking agent/lyoprotectant added before lyophilization. In one example, the formulation is a saline solution containing 3.5% sodium alginate, 0.5% HPMC, 5-10 mg/mL poly IC with a pH value at 6-7, which can be injected intratumorally to treat cancer at 0.5-2 mL/tumor. In one example, the formulation is a saline solution containing 5% sodium alginate and 1-2 mg/mL imiquimod with a pH value at 6-7, which can be injected intratumorally at 0.5-2 mL/tumor to treat cancer. In one example, the formulation is a saline solution containing 5% sodium alginate, 3-6 mg/mL poly IC and 1-2 mg/mL imiquimod with a pH value at 6-7, which can be injected intratumorally to treat cancer. Poloxamer at a concentration (e.g. 20-25%) that can produce in situ gelling can also be used instead of alginate.

In one example, the formulation is a saline solution containing 6% sodium alginate and 5-10 mg/mL poly IC or 2-6 mg/mL imiquimod or 5-10 mg SD-101 or their combination, with pH value adjusted to 6-7 and osmolarity adjusted to physiological value. The reconstituted Herceptin solution containing 21 mg/mL of trastuzumab according its reconstitution method in its product insert is mixed with this formulation at 1:1 v/v ratio to generate the final solution to be injected intratumoral (e.g. 0.1 ml-0.5 mL/cm3 tumor volume) to HER2+ tumor. It can also be applied to the surgical area where tumor is removed, optionally with sequential addition of Ca′ salt solution to promote gelling in situ as previously described. In another example, 3 mg/mL imiquimod or 1 mg/mL 3M-052 is added in above formulation to be mixed with antibody. In another example, poly IC is replaced with 2-6 mg/mL imiquimod or 0.5-2 mg/mL R848. In another example, said formulations is mixed with Bavencio 20 mg/mL concentrate at 1:1 ratio to treat PD-L1 positive tumor.

When the formulation is applied right after surgery to the area where tumor is removed, the formulation can be a ready to use gel instead of an in situ gelling system. It can be in a shape suitable to be applied to the tumor removed area such as a disk, a wafer, a film, a sheet, a rod, a flake, a plate and etc. Examples of gel system can be alginate with Ca′, collagen, gelatin, cross-linked hyaluronic acid that can from stable hydro gel. In some embodiments, the composition/formulation contains therapeutically effective amount of TLR agonist or STING agonist or anti-tumor antibody or immune boosting protein or chemotherapy drug or their combinations in a gel matrix consisting of alginate with Ca²⁺ or collagen or gelatin at the concentration that can form hydro gel in vitro. The hydrogel can be applied to tumor removed area to slowly release the drugs trapped in the gel to treat cancer and enhance immunity against cancer. It can be cut to a suitable shape and size to be used. In one example, the formulation is a hydro gel film containing 3.5% sodium alginate, 2-6 mg/g poly IC, 1% CaCl₂). 1-3 g of the film can be applied to the tumor removed area. In another example, the formulation is a hydro gel film containing 5% sodium alginate, 1% CaCl₂), 5 mg/g poly IC and 1-2 mg/g imiquimod. In another example, the formulation is a hydro gel film containing 5% sodium alginate, 1% CaCl₂), 3 mg/g poly IC, 1 mg/g imiquimod or 0.1 mg/g R848 and optional 3 mg/mL CPG ODN such as SD-101. In some embodiments, said formulations further comprise one or more therapeutical antibody against cancer at therapeutically effective amount such as 1-10 mg/g. In one example, the formulation is a hydro gel film containing 3.5% sodium alginate, 1% CaCl₂), 10 mg/g Trastuzumab or antibody against PD-L1 or antibody against EpCAM or their combination. In another example, the formulation is a hydro gel disk containing 3% sodium alginate, 1% calcium gluconate, 3 mg/g poly IC, 1 mg/g imiquimod and 10 mg/g trastuzumab or 10 mg/g atezolizumab. In some embodiments, said formulations further comprise one or more therapeutical proteins that can boost immunity against tumor at therapeutically effective amount. In one example, the formulation is a hydro gel film containing 3.5% sodium alginate, 1% CaCl₂), 1-10 MIU/g IL-2. In one example, the formulation is a hydro gel film containing 3.5% sodium alginate, 1% CaCl₂), 5 MIU/g IL-2 and 5 mg/g anti EpCAM antibody having tandem Fc or CH2 repeat in a format selected from those shown in FIGS. 1-4. In another example, the formulation is a hydro gel disk containing 3% sodium alginate, 1% CaCl₂), 3 mg/g poly IC, 1 mg/g imiquimod and 5 MIU/g IL-2. In some embodiments, said formulations further comprise one or more chemotherapy drug at therapeutically effective amount. In one example, the formulation is a hydro gel film containing 3.5% sodium alginate, 1% CaCl₂), 5-50 mg/g chemotherapy drug such as cisplatin or paclitaxel or carmustine or doxorubicin or their combinations. In another example, the formulation is a hydro gel disk containing 3% sodium alginate, 1% CaCl₂), 3 mg/g poly IC, 1 mg/g imiquimod and 5 mg/g oxaliplatin. In another example, the formulation is a hydro gel disk containing 2% sodium alginate, 1% CaCl₂), 3 mg/g poly IC, 1 mg/g imiquimod and 10 mg/g paclitaxel and 5 mg/g oxaliplatin and optional solubility enhancing excipient such as 0.1% tween-20 or 5% propylene glycol. In another example, the formulation is a hydro gel membrane containing 5% sodium alginate, 1% CaCl₂), 3 mg/g poly IC, 1 mg/g imiquimod and 10 mg/g paclitaxel and 5 mg/g Herceptin to treat HER2+ cancer. In another example, the formulation is a hydro gel wafer containing 5% sodium alginate, 1% CaCl₂), 3 mg/g poly IC, 0.2 mg/g R848, 20 mg/g doxorubicin, 5 MIU/g IL-2 and 5 mg/g atezolizumab. In some embodiments, said hydro gel formulation use collagen or gelatin at the concentration that can form hydrogel as gelling agent instead of alginate+CaCl₂) used above. For example, 0.5% 5% collagen can be used to replace the alginate+CaCl₂) in above formulations to form hydrogel. In another example, 5%˜15% gelatin can be used to replace the alginate+CaCl₂) in above formulations to form hydrogel.

The hydrogel can be dried into a film or membrane and then applied to the site where tumor is surgically removed. Excipient that can improve flexibility and elasticity can be added to the formulation such as glycerin or PEG. In one example, a hydro gel is prepared by mixing 5% sodium alginate containing 6% glycerin, 3 mg/g poly IC, 1 mg/g homogenized imiquimod and 10 mg/g trastuzumab (or 10 mg/g atezolizumab) with half volume of 3% CaCl₂) to form a gel. After the gel is formed, the liquid on top of the gel is removed and the gel is dried under vacuum to remove >60% of water to form a flexible film to be applied to patient having HER2+ tumor (or PD-L1 positive tumor if atezolizumab is used in the film.

### Embodiments Type 2

The current invention also discloses composition, formulations and its use to treat cancer, which comprises tumor inactivating antibody and immunity boosting agent in a self-gelling matrix, which can be injected into the tumor or injected into or proximal to the tumor draining lymph node, or be applied to the area where tumor is removed after surgery to treat cancer. In some embodiment, the tumor inactivating antibody is therapeutic antibody used to treat cancer. In some embodiment, the therapeutic antibody used to treat cancer targets the antigen on cancer cell surface. In some embodiment, said therapeutic antibody targeting cancer cell surface antigen is selected from HER2 targeting antibody such as trastuzumab, EGFR targeting antibody such as cetuximab, antibody against EpCAM, catumaxomab, antibody against PD-L1 or their combinations. Other antibody targeting cancer cell surface antigen described in prior application and current applications can also be used. In some embodiment, the concentration of antibody in the composition and formulation of liquid form is between 2 mg/ml to 50 mg/ml. In some embodiment the immunity boosting agent is one or more TLR agonist or STING agonist or their combination. In some embodiment the TLR agonist is imiquimod or R848 or poly IC or 3M-052 or their combinations. Preferably the formulation is an injectable liquid. In some embodiment, the self-gelling matrix is alginate and contains no calcium salt. In some embodiment, the formulation contains alginate and low concentration of calcium salt that will not cause gelling before being injected. In some embodiment, composition and formulation of liquid form comprise 2˜9% sodium alginate. In some embodiment, the self-gelling matrix is poloxamer. In some embodiment additional gelling enhancing polymer such as hyaluronic acid, HPMC, chitosan, CMC (e.g. 0.5%˜2.5% w/w) can also be incorporated in the liquid formulation. In some embodiments, therapeutically effective amount of proteins that can activate/boost the function of immune system such as granulocyte macrophage colony-stimulating factor, FLT3L, IFN-α, IFNγ, anti-tumor interleukin such as interleukin-2, or their derivatives such as PEGylated derivative; can also be incorporated in the formulation. The composition/formulation in liquid from can also be lyophilized and reconstituted before use. A bulking agent/lyoprotectant can be added such as 3% sucrose before lyophilization. In certain embodiments, the formulations is a solution containing about 5-20 mg/mL antibody against tumor cell surface antigen (e.g. trastuzumab, cetuximab, antibody against EpCAM such as catumaxomab, antibody against PD-L1 such as avelumab), 1-5 mg/mL imiquimod suspension, and/or 2-10 mg/mL poly IC in 2-6% sodium alginate or 20% Poloxamer 407. In one example, the formulation to treat HER2 positive cancer is a solution containing about 5 mg/mL trastuzumab, 2 mg/mL imiquimod and optional 4 mg/mL poly IC, 2% sodium alginate with pH between 6-8 and osmolarity adjusted to physiological osmolarity with sucrose. In another example, the formulation is a solution containing about 10 mg/mL cetuximab, 3 mg/mL imiquimod, 5 mg/mL poly IC, 3.5% sodium alginate with pH between 6-8 and osmolarity adjusted to physiological osmolarity with NaCl. In another example, the formulation is a solution containing about 5 mg/mL atezolizumab and 5 mg/mL catumaxomab, 3 mg/mL imiquimod, 5 mg/mL poly IC, 1% HPMC, 3.5% sodium alginate with pH between 6-8 and osmolarity adjusted to 250˜350 mOsm/kg with mannitol or NaCl. In another example, the composition/formulation to treat PD-L1 positive cancer is a solution containing about 10 mg/mL avelumab, 2 mg/mL imiquimod, 4 mg/mL poly IC, 5 MIU/mL IL-2 such as aldesleukin, optional 2 mg/mL SD-101, 3.5% sodium alginate with pH between 6-8 and osmolarity adjusted close to physiological osmolarity with NaCl. In another example, the composition/formulation is a solution containing about 10 mg/mL IgG1 or IgM antibody against EpCAM, 5 mg/mL poly IC, 5 mg/mL CPG ODN such as SD-101, 3.5% sodium alginate with pH between 6-8 and osmolarity adjusted to 250˜350 mOsm/kg with NaCl. In another example, the composition/formulation is a solution containing about 30 mg/mL anti EpCAM antibody having tandem Fc or CH2 repeat in a format selected from those shown in FIGS. 1-4, 5 mg/mL poly IC, 5 mg/mL CPG ODN such as SD-101 in 3.5% sodium alginate with pH between 6-8 and osmolarity adjusted to 250˜350 mOsm/kg with NaCl or in 20% Poloxamer 407. In another example, the composition/formulation is a solution containing about 20 mg/mL bispecific antibody targeting EpCAM and APC surface marker (e.g. DC-SIGN) in a format selected from those shown in FIGS. 5-6, 5 mg/mL poly IC, 5 mg/mL CPG ODN such as SD-101, 3.5% sodium alginate with pH between 6-8 and osmolarity adjusted to 250˜350 mOsm/kg with NaCl. In another example, the composition/formulation is a solution containing about 20 mg/mL antibody-TLR agonist conjugate targeting EpCAM such as those shown in FIGS. 7-10, optional 5 mg/mL poly IC, optional 5 mg/mL CPG ODN such as SD-101, 3.5% sodium alginate with pH between 6-8 and osmolarity adjusted to 250˜350 mOsm/kg with NaCl, or in 20% Poloxamer 407. In some embodiments, additional chemotherapy drug can also be incorporated.

### Embodiments Type 3

The current invention also discloses composition, formulations and its use to treat cancer, which comprises chemotherapy drug and/or other small molecule cytotoxic agent and said immunity boosting agent such as said vaccine adjuvant type agent in a self-gelling matrix such as temperature triggered in situ gelling system, which can be injected into the tumor or injected into or proximal to the tumor draining lymph node, or be applied to the area where tumor is removed after surgery to treat cancer. Preferably the formulation is an injectable liquid. In some embodiment the immunity boosting agent is one or more TLR agonist or STING agonist or their combination. In some embodiment the TLR agonist is imiquimod or R848 or poly IC or 3M-052 or their combinations. Examples of the self-gelling matrix are previously in the current application such as xyloglucan, poloxamines, poloxamer (trade names Synperonics, Pluronic, and Kolliphor). For example, 15-25% Pluronic F127 or Poloxamer 407 or Poloxamer P188 or their combination can be used in said composition/formulation as the self-gelling matrix. Optionally additional gelling enhancing polymer (e.g. 0.5%˜2.5% w/w) such as hyaluronic acid, HPMC, carbomer, chitosan, CMC and small molecule such as fructose (5-10%) and sodium citrate tribasic dihydrate (SC, 1-5%) can also be incorporated in the liquid formulation. The composition/formulation in liquid from can also be lyophilized and reconstituted before use. A bulking agent/lyoprotectant can be added such as 3% sucrose before lyophilization.

Example of the chemotherapy drugs include alkylating agents (such as cyclophosphamide, uramustine, carmustine and usulfan), antimetabolites (such as methotrexate and fluorouracil), anti-microtubule agents (such as paclitaxel, vindesine, and vinflunine), topoisomerase inhibitors (such as irinotecan and topotecan), platinum-based antineoplastic (such as cisplatin, oxaliplatin, and carboplatin) and cytotoxic antibiotics (such as anthracyclines, bleomycins, mitomycin C, mitoxantrone, and actinomycin) and can be used between 0.5˜5% w/w in the formulation. Preferably the chemotherapy drug has higher cytotoxicity against tumor cells than against immune cells, especially the antigen presenting cell such as dendritic cell. Preferably their IC 50 or CC50 ratio is <0.5. More preferably their IC 50 or CC50 ratio is <0.2. Examples of other small molecule cytotoxic agent include acid or base (e.g. 0.1˜1M pH=2 lactic acid buffer, 0.1˜1M pH=10 NaCO3 buffer), organic solvent (e.g. 75% ethanol, DMF, DMSO, acetone), cell inactivating peptide, detergent/surfactant as described previously. Cell inactivating peptide and antibiotics such as polymyxin are also detergent like compound, which can be used in the current invention. Examples of the detergent that can be used include anionic detergents, cationic detergents, non-ionic detergents and zwitterionic detergents such as alkylbenzenesulfate, alkylbenzenesulfonates, bile acids, deoxycholic acid, quaternary ammonium type detergents, tween, triton, CHAPS, SLS, SDS, SLES, DOC, NP-40, cetrimonium bromide (CTAB), cetylpyridinium chloride (CPC), benzalkonium chloride (BAC), benzethonium chloride (BZT), dimethyldioctadecylammonium chloride and dioctadecyldimethylammonium bromide (DODAB) as long as they can effectively lyse the tumor cell in vivo.

Additional previously described therapeutically effective amount cancer treating antibody, agent that can activate/boost the function of immune system, agent that can inactivate MDSC, agent that can inactivate Treg can also be incorporated in the formulation.

In one example, a formulation containing chemotherapy drug only is a liquid containing 18-22% poloxamer 407 or Pluronic F127, 3-25 mg/g chemotherapy drug such as cisplatin or paclitaxel or carmustine or doxorubicin or their combinations in water, which can be injected into tumor at 0.1-0.5 ml/cm³ tumor volume or applied to the tumor removed area to treat cancer. In another example, a formulation containing TLR agonist only is a liquid containing 17-20% poloxamer 407 or Pluronic F127, 2-6 mg/g poly IC, 1-3 mg/g imiquimod in water, which can be injected into tumor to treat cancer at 0.1-0.5 ml/cm³ tumor volume or applied to the tumor removed area to treat cancer. In another example, the formulation is a liquid containing 17-20% poloxamer 407, 3 mg/g poly IC, 1 mg/g imiquimod and 5 mg/g oxaliplatin in water, which can be injected into tumor at 0.1-0.5 ml/cm³ tumor volume or applied to the tumor removed area to treat cancer. In another example, the formulation to treat cancer is a liquid containing 20-25% Pluronic F127, 0.5% crosslinked hyaluronic acid, 3 mg/g poly IC, 1 mg/g imiquimod and 10 mg/g paclitaxel and 5 mg/g oxaliplatin in water. In another example, the formulation to treat cancer is a liquid containing 15-18% Pluronic F127, 1-5% HPMC or MC or CMC, 3 mg/g poly IC, 0.2 mg/g R848 or 0.1 mg/g 3M-052, 20 mg/g doxorubicin in water. In another example, the formulation to treat cancer is a liquid containing 15-19% Pluronic F127, 5-10 mg/mL Irinotecan, 1-5 mg/mL imiquimod suspension, optional 2-10 mg/mL poly IC, optional 5% SDS and 2-6% sodium alginate in water. In another example, the composition/formulation is a solution containing optional 10 mg/mL avelumab, optional 5% bile acid, 0.5% vindesine, 2 mg/mL imiquimod, 4 mg/mL poly IC, optional 2 mg/mL SD-101 and 17-20% poloxamer 407 in water. In another example, the composition/formulation is a solution containing about 10 mg/mL IgG1 or IgM antibody against EpCAM, 5 mg/mL poly IC, 5 mg/mL CPG ODN such as SD-101, 5-10 mg/mL docetaxel and 20% poloxamer 407 in water.

### Embodiments Types 4

The composition, formation and use in this embodiments set are the same as those in embodiments set 3 except biodegradable water insoluble polymer dissolved in organic solvent is used as in situ gelling system to replace the temperature triggered in situ gelling polymer. The current invention discloses compositions, formulations and its use to treat cancer, which comprises chemotherapy drug and/or other small molecule cytotoxic agent and said immunity boosting agent (drug) such as said vaccine adjuvant type agent in a biodegradable water insoluble polymer matrix dissolved in organic solvent, which can be injected into the tumor or injected into or proximal to the tumor draining lymph node, or be applied to the area where tumor is removed after surgery to treat cancer. In some embodiment the immunity boosting drug is one or more TLR agonist or STING agonist or their combination. In some embodiment the TLR agonist is imiquimod or R848 or poly IC or 3M-052 or their combinations.

Examples of the biodegradable water insoluble polymer matrix dissolvable in organic solvent are described previously such as PLA, PLGA, PCL, PGA, prolifeprospan (e.g. prolifeprospan 20) or PHB. These polymer can be dissolved in biocompatible water miscible organic solvent such as N-methyl pyrrolidone or DMSO as matrix to load the drug, the drug can be dissolved/dispersed in the said polymer solution such as PGA or PLGA solution (e.g. 10% 50% PLGA in N-methyl pyrrolidone or DMSO), or two components (the drug and the polymer solution) are combined immediately before injection. Alternatively, a mixture of water insoluble polymer with the drugs in their dry form is stored in a separate vial, and suitable amount of said organic solvent is added to the vial to dissolve them right before use. In some embodiments, 50:50 lactide/glycolide PLGA or PLGA with lower lactide content can be used, e.g. 10:90 lactide/glycolide PLGA. In some embodiments, prolifeprospan 20 or PGA is used. When this formulation is injected into the body or applied to the tumor removed area, the water miscible organic solvent dissipates and water penetrates into the organic phase. This leads to phase separation and precipitation of the polymer forming a drug depot at the site as a sustained release implant type composition. Optionally additional gelling/releasing adjusting agent such as PEG, poloxamer can also be incorporated in the formulation. Addition of poloxamer may enhance the gelling of the formulation and the addition of PEG can increase the drug release rate. Polymer (e.g. 0.5%˜2.5% w/w) such as hyaluronic acid, HPMC, carbomer, chitosan, CMC can also be incorporated in the liquid formulation.

Examples of the chemotherapy drugs are described previously such as those in embodiments type 3 and can be used between 0.5˜5% w/w in the formulation. Other small molecule cytotoxic agent described previously such as those in embodiments type 3 can also be used. Additional previously described therapeutically effective amount cancer treating antibody, agent that can activate/boost the function of immune system, agent that can inactivate MDSC, agent that can inactivate Treg can also be incorporated in the formulation.

In one example, a formulation containing chemotherapy drug only is a liquid containing 25-45% 50:50 lactide/glycolide, 3-25 mg/g chemotherapy drug such as cisplatin or paclitaxel or carmustine or doxorubicin or their combinations in N-methyl pyrrolidone (NMP) or DMSO, which can be injected into tumor at 0.1-0.5 ml/cm³ tumor volume or applied to the tumor removed area to treat cancer. In another example, a formulation containing TLR agonist only is a liquid containing 35% 50:50 lactide/glycolide, 2-6 mg/g poly IC, 1-3 mg/g imiquimod in NMP or DMSO, which can be injected into tumor to treat cancer at 0.1-0.5 ml/cm³ tumor volume or applied to the tumor removed area to treat cancer. The formulation can also be a two component format, one is a solution of 25-45% 50:50 lactide/glycolide in NMP or DMSO, another is the dry form of other drugs, the two components are mixed together before use. Alternatively, one vial contains the dry form of lactide/glycolide and the drugs at a desired ratio (e.g. PLGA 30-500 mg: chemotherapy drug 3-25 mg:TLR agonist 1-6 mg) and then mix with suitable amount of NMP or DMSO (e.g. 0.6 mL-1.5 ml) before use to generate a solution containing target amount of drugs and PLGA.

In another example, the formulation is a liquid containing 40% PGA, 3 mg/g poly IC, 1 mg/g imiquimod and 5 mg/g oxaliplatin in NMP or DMSO, which can be injected into tumor at 0.1-0.5 ml/cm³ tumor volume or applied to the tumor removed area to treat cancer. In another example, the formulation to treat cancer is a liquid containing 30-50% prolifeprospan, 5 mg/g poly IC, 1 mg/g imiquimod and 10 mg/g paclitaxel and 5 mg/g oxaliplatin in NMP or DMSO. In another example, the formulation to treat cancer is a liquid containing 40% prolifeprospan 20, 2% HPMC or MC or CMC, 3 mg/g poly IC, 0.2 mg/g R848 or 0.1 mg/g 3M-052, 20 mg/g doxorubicin in NMP or DMSO. In another example, the formulation to treat cancer is a mixture of 400 mg prolifeprospan 20, 3 mg poly IC, 0.2 mg R848 or 0.1 mg 3M-052, 10 mg doxorubicin in dry form, which is to be mixed with organic solvent to generate the final formulation before use. In another example, the formulation to treat cancer is a liquid containing 35% 10:90 lactide/glycolide PLGA, 5-10 mg/mL irinotecan, 1-5 mg/mL imiquimod suspension, optional 2-10 mg/mL poly IC, optional 5% SDS in NMP or DMSO. In another example, the composition/formulation is a solution containing 40% 25:75 lactide/glycolide PLGA, optional 10 mg/mL avelumab, optional 5% bile acid, 0.5% vindesine, 2 mg/mL imiquimod, 4 mg/mL poly IC, optional 2 mg/mL SD-101 and optional 15-20% poloxamer 407 in NMP or DMSO. In another example, the composition/formulation is a solution containing about 35% 60:40 lactide/glycolide PLGA, 5 mg/mL poly IC, 5 mg/mL CPG ODN such as SD-101, 5-10 mg/mL docetaxel and 5% PEG6000 in NMP or DMSO.

When the compositions/formulations/examples/embodiments in Embodiments types 4 are to be applied to the site where tumor is removed during surgery to treat cancer, it can be in dry form such as a drug containing film or other shape (e.g. disk, plate, membrane) implant and then applied to the tumor removed site, therefore no organic solvent (e.g. NMP or DMSO) is required in the dry form. It is essentially drugs in a solid state polymer matrix such as PLGA with a suitable shape to be placed locally. For example, a PLGA disk contains 20 mg/g poly IC, 5 mg/g imiquimod and 50 mg/g paclitaxel.

In some embodiments of the current inventions, the in-situ gelling matrix rely on the gelling formation at the presence of di or trivalent cationic ion or polycationic molecule, such as pectin, alginate, hyaluronic acid and gellan gum as described previously. In some embodiments, low water solubility di/trivalent/polycationic compound (e.g. low water solubility divalent cation salt such as calcium carbonate, calcium phosphate, dicalcium phosphate, calcium silicate, CaSO₄, ZnCO₃, BaCO₃, BaSO₄ or their combination), can be added to these type of in-situ gelling formulation right before injection, which can be injected into the tumor when it is still in low viscosity state and forms gel slowly in vivo. In some embodiments, the final concentration of the low water solubility calcium salt or zinc salt or barium salt in the final drug loaded formulation to be injected is between 0.3-10%. A sustained release formulation that can release these cationic ion such as calcium ion or zinc ion or their combination slowly is also considered as a low water solubility cationic ion compound, e.g. nano or microparticle that can release its encapsulated calcium ion in 15 min-1 hour when it is in contact with water. Therefore the current invention also provide a kit to treat cancer, which comprise two separate components in different containers to be mixed together right before injection. The two components can also be placed in one container if they are all in solid form (e.g. both being dried such as lyophilized). One component contains low water solubility di/trivalent/polycationic compound that can enhance the gelling of the second component, either in solid dosage form or liquid form. The second component is a drug loaded in situ gelling formulation such as those described previously, or similar formulation with higher concentration of in situ gelling agent and drug, which will compensate the dilution factor and provide same concentration of drug/gelling agent as those in the formulation previously described after it is mixed with the first component. In one example, the kit contains two components, one is 6% calcium carbonate or CaSO4 or ZnCO3 or calcium phosphate or dicalcium phosphate suspension in water, another is a previously drug loaded formulation such as a saline solution containing 4% sodium alginate, 5-10 mg/mL poly IC and 2-5 mg/mL imiquimod. The two component will be mixed together at 1:1 v/v ratio and the mixture will be injected into the tumor to treat cancer. The two components can also be mixed together with a 21 mg/mL of trastuzumab (Herceptin) solution at 1:1:0.5 ratio to be injected into the HER2+ tumor to treat cancer. In another example, said drug loaded second component further contains 20 mg/mL anti PD-L1 antibody. Additional viscosity enhancing polymer such as starch, cellulose, methyl cellulose, HPMC can also be incorporated into the first component at suitable concentration such as 0.1-5% w/w.

In some embodiments, solution of water soluble (e.g. solubility >0.5% at room temperature) di/trivalent/polycationic compound (e.g. CaCl₂), calcium gluconate, zinc chloride or gluconate, Ca-EDTA, ferrous chloride FeCl₂, ferrous gluconate, FeCl₃, ferric gluconate, BaCl₂, barium gluconate, ornithine or its derivatives, lysine or its derivatives such as lysine ethyl ester, arginine or its derivatives such as arginine ethyl ester, chitosan, poly lysine, poly arginine, poly ornithine or their combination) can be injected into the same tumor right before or right after the tumor is injected with a drug loaded in-situ gelling formulation to improve the gelling effect. They can be also be co-injected using a dual syringe system. In some embodiments, the concentration of water soluble di/trivalent/polycationic compound in the solution is between 0.2-10%. In some embodiments, the concentration of the water soluble calcium salt in the solution is between 0.2-6%. When it is injected before the injection of the drug loaded formulation, it will prime the tumor with higher concentration of Ca²⁺ ion than physiological Ca²⁺ ion level to provide better in situ gelling effect. Therefore the current invention also provide a kit to treat cancer, which comprise two separate components in different containers to be sequentially injected into the tumor or co-injected into tumor using a dual syringe system. One component contains water soluble di/trivalent/polycationic compound that can enhance the gelling of the second component, either in solid dosage form or liquid form. The second component is a drug loaded in situ gelling formulation such as those described previously, or similar formulation with higher concentration of in situ gelling agent and drug, which will compensate the dilution factor and provide same concentration of drug/gelling agent as those in the formulation previously described after it is mixed in vivo with the first component. In one example, the kit contains two components, the first one is 0.5-5% CaCl₂) or calcium gluconate or chitosan or lysine or arginine in water with pH value between 5-8, the second one is a previously drug loaded formulation is a saline solution containing 4% sodium alginate, 5-10 mg/mL poly IC and 2-5 mg/mL imiquimod with optional 10 mg/mL trastuzumab or IgG1 antibody against EpCAM. Additional viscosity enhancing polymer such as starch, cellulose, methyl cellulose, HPMC can also be incorporated into the first component at suitable concentration such as 0.1-5% w/w. In one example, component 1 is a pH 7 solution containing 0.5% HPMC, 1% CaCl₂) or 2% calcium gluconate or 1.5% lysine or 1% chitosan, osmolarity adjusted with NaCl to be close to physiological value, is injected to the tumor at 1-5 mL depend on the size of the tumor. Next the drug loaded alginate containing solution component 2 is injected into the same tumor to treat the subject in need. The component 1 can also be injected after the injection of component 2. Component 1 and 2 can also be placed in one syringe for injection, separated by a biologically and pharmaceutically acceptable liquid solution as a buffer layer to prevent them being mixed together inside the syringe. In some embodiment, the buffer layer liquid can be a liquid having high viscosity (e.g. >500 cps, or >2000 in other embodiments) to reduce permeation and contamination between components 1 and 2. For example, it can be glycerin, or a pH 7 solution containing viscosity enhancing polymer such as starch, cellulose, methyl cellulose, HPMC, HA at 0.3-3% w/w, osmolarity adjusted to 250˜350 mOsm/kg with NaCl. In one example, a syringe containing 2 ml of component 1 and 2 ml component 2 with 1 ml buffer liquid in the middle is used for intratumoral injection. Therefore, the kit to treat cancer can further comprise a 3rd component, which is a buffer layer liquid as described above.

In some embodiments, the low water solubility divalent cation salt (e.g. calcium carbonate, calcium phosphate, dicalcium phosphate, calcium silicate, CaSO₄, ZnCO3, BaCO3, BaSO4 or their combination) or Ca-EDTA containing formulation can further mix with an agent that can slowly release these cation from the low solubility salt or from Ca-EDTA complex right before administrate the formulation to a subject in need, which will cause the gelling slowly in vivo. Example of these agent can be selected from D-glucono-delta-lactone (GDL), L-glucono-delta-lactone, D-erythronolactone, L-erythronolactone, D-glucuronolactone, L-glucuronolactone, D-galactono-gamma-lactone, L-galactono-gamma-lactone, D-xylono-gamma-lactone, L-xylono-gamma-lactone, D-gulono-gamma-lactone, L-gulono-gamma-lactone 3, D-glucono-gamma-lactone and L-glucono-gamma-lactone. Those lactone can hydrolyze slowly in water to release acid which will release the free divalent cation in to water to cause gelling. Higher pH increase hydrolysis speed and lower pH reduce hydrolysis speed which will in turn affect the gelling time. The pH of the formulation can be adjusted accordingly (e.g. pH5-8) to achieve the desired gelling time. The ratio of these agent vs divalent cation salt can be between 1:5 to 5:1 molar ratio. For example, when GDL and CaCO₃ or CaCO₃+CaSO₄ mixture are used in the formulation, their molar ratio can be 1:2 or 1:1 or 2:1. In one example, 5 mL drug loaded formulation containing 2% sodium alginate is mixed with 0.1 g CaCO₃ powder and then mixed with 0.05 g GDL powder. After stirring, the final formulation is intratumorally injected to treat cancer, which will form gel in vivo. In another example, 5 mL of saline solution containing 4% sodium alginate, 5 mg/mL poly IC and 2 mg/mL imiquimod with optional 10 mg/mL trastuzumab or IgG1 antibody against EpCAM is the first formulation, it is mixed with a dry powder mixture (the 2^{nd} formulation) of 0.1 g CaCO₃ (or ZnCO₃), 0.1 g CaSO₄ and 0.1 g GDL (or L-gulono-gamma-lactone). After stirring, it is injected intratumorally to treat cancer. Additional viscosity enhancing polymer can be incorporated in the formulation, which will reduce the gelling speed.

The drug loaded sustained release formulation in the current inventions can be either in-situ gelling formulation or nano/microparticles based formulation or their combinations. The drug (e.g. TLR agonist, chemotherapy drug, antibody or other cancer cell inactivating agent) can be encapsulated in nanoparticle or microparticle as a sustained release form to be injected intratumorally or injected into or proximal to the tumor draining lymph node or applied to the site where tumor is removed during surgery to treat cancer. Suitable size of microparticle can be 1 μm-100 μm in diameter. In some embodiments, the size of microparticle is between 1 μm-30 μm. In some embodiments, the size of microparticle is between 5 μm-20 μm. In some embodiments, the drug encapsulated nanoparticle or microparticle is made of synthetic biodegradable polymer described previously, such as PLGA. In some embodiments, the drug encapsulated nanoparticle or microparticle is made of polysaccharide such as alginate based particle which use polysaccharide such as alginate to form the matrix of particle. For example, it can be chitosan-calcium alginate gel nano/microsphere such as those described in patent No. CN1628861, or chitosan-alginate nano/microsphere, or calcium alginate nano/microsphere such as those described in patent No. CN107057085, or sodium alginate-calcium carbonate hybrid microparticles such as those described in patent No. CN102286155, or calcium phosphate/calcium alginate hybrid microspheres such as those described in patent No. CN101081911. These drug loaded particles can be readily prepared by using a alginate solution containing drug by adopting or modifying the protocols described in prior publications.

In one example, dissolve 100 mg of sodium alginate in 10 ml of distilled water, heat in a water bath at 40° C., add 300 mg solution of sodium phosphate and stir in a water bath at for 30 minutes, add 50 mg poly IC, 20 mg imiquimod, 50 mg anti-PD-L1 IgG1, then slowly add 5 ml 1% calcium chloride solution and stirred in and hour, then centrifuged, to obtain drug loaded alginic acid-calcium phosphate microparticles. In another example, drug loaded alginate-Ca particle is prepared by adding drug containing 2.0% (w/v) sodium alginate solution (e.g. 10 mg/mL poly IC, 2 mg/mL imiquimod and 10 mg/mL antibody in alginate solution) using electrostatic drop method and vigorous stirring to 1% CaCl₂) solution as the gel bath to obtain calcium alginate gel microspheres, then the drug loaded calcium alginate gel microspheres is coated with 0.7% (w/v) chitosan solution by mixing them at 1:10 v/v ratio. Then centrifuged to obtain drug loaded alginic-calcium-chitosan microparticles. In the above two examples, different drugs are encapsulated in one microparticle (multiple species of drugs in each microparticle). Alternatively, different drug can be encapsulated in different microparticle (one species of drug in each microparticle) and the formulation contains the mixture of these different microparticle therefore the formulation contains mixtures of different drugs. In the above two examples, alginate is the matrix of microparticle. Alternatively synthetic biodegradable polymer previously described such as PLGA can be used as matrix of drug loaded nano or microparticle. In some embodiments, different drugs (TLR agonist, chemotherapy drug and antigen-lipid conjugate) are encapsulated in one particle. For example, each PLGA microparticle (5 μm-20 μm) contains 3% polyIC, 1% imiquimod, 5% paclitaxel and optional 5% L-rhamnose-cholesterylamine conjugate. Alternatively, each PLGA microparticle contains only one type of above drugs and mixture of different beads are used in the formulation.

Unless otherwise defined, all technical and scientific terms used herein have the same meaning as commonly understood by one of ordinary skill in the art to which this invention belongs. All patents and publications mentioned in this specification are indicative of the level of those skilled in the art to which the invention pertains. All patents and publications are herein incorporated by reference to the same extent as if each individual publication was specifically and individually indicated to be incorporated by reference. The inventions described above involve many well-known chemistry, instruments, methods and skills. A skilled person can easily find the knowledge from text books such as the chemistry textbooks, scientific journal papers and other well-known reference sources.

1. A pharmaceutical composition comprising a cancer cell inactivating agent, an immune activity enhancing agent and an in situ gelling polymer in an in situ gelling system, wherein the in situ gelling system forms gel in vivo.

2. The pharmaceutical composition of claim 1, wherein the cancer cell inactivating agent is selected from chemotherapy drug, antibody against tumor cell surface marker or antigen-lipid conjugate.

3. The pharmaceutical composition of claim 1, wherein the immune activity enhancing agent is a Toll-like receptor agonist.

4. The pharmaceutical composition of claim 1, wherein the immune activity enhancing agent is a STING agonist.

5. The pharmaceutical composition of claim 1, wherein the in situ gelling polymer is selected from alginate, poloxamer or PLGA.

6. A method of treating tumor cell, comprising administering to a patient in need thereof a therapeutically effective amount of a mixture of a cancer cell inactivating agent, an immune activity enhancing agent and an in situ gelling polymer in an in situ gelling system.

7. The method of claim 6, wherein the cancer cell inactivating agent is selected from chemotherapy drug, antibody against tumor surface marker or antigen-lipid conjugate.

8. The method of claim 6, wherein the immune activity enhancing agent is selected from Toll-like receptor agonist, STING agonist or a mixture thereof.

9. The method of claim 6, wherein the in situ gelling polymer is selected from alginate, poloxamer or PLGA.

10. The method of claim 6, wherein the administering is via intratumoral injection.
